(19) United States
(12) Patent Application Publication
(10) Pub. No.: US 2020/0108129 A1
(43) Pub. Date: Apr. 9, 2020

(54) CLOSTRIDIAL TOXIN - HYALURONIC ACID COMPOSITIONS

(71) Applicant: ALLERGAN, INC., Irvine, CA (US)

(72) Inventors: Cindy WU, Irvine, CA (US); James CUNNINGHAM, Irvine, CA (US); Marianne DO, Irvine, CA (US)

(21) Appl. No.: 16/571,014

(22) Filed: Sep. 13, 2019

Related U.S. Application Data
(60) Provisional application No. 62/731,063, filed on Sep. 13, 2018.

(51) Int. Cl. A61K 38/48; A61K 31/728; A61K 47/26; A61K 47/34; A61K 47/20

(52) U.S. Cl. CPC A61K 38/4893; C12Y 304/24069; A61K 47/20; A61K 47/26; A61K 47/34; A61K 31/728

(57) ABSTRACT

Pharmaceutical compositions that extend the effect and duration of a Clostridial toxin active ingredient are described. The compositions can be liquid or solid compositions, and comprise a non-cross-linked hyaluronic acid or salt thereof as described in the application, a surfactant and an antioxidant. In some embodiments, the compositions comprise a surfactant selected from a poloxamer and a polysorbate; an antioxidant selected from methionine, N-acetyl cysteine, ethylenediaminetetraacetic acid and combinations thereof, and, optionally, a tonicity agent and/or a lyoprotector selected from, for example, trehalose, sucrose.

### CROSS-REFERENCE TO RELATED APPLICATIONS

This application claims the benefit of U.S. Provisional Application No. 62/731,063, filed Sep. 13, 2018, incorporated herein by reference in its entirety.

### TECHNICAL FIELD

The present disclosure relates to pharmaceutical compositions comprising a Clostridial toxin active ingredient and a non-cross-linked hyaluronic acid or salt thereof, where the weight average molecular weight of a non-cross-linked hyaluronic acid or salt thereof is from 250 kDa to 2.4 MDa or from 4.6 MDa to 8 MDa.

### BACKGROUND

A pharmaceutical composition is a formulation which contains at least one active ingredient (such as a Clostridial toxin) as well as, for example, one or more excipients, buffers, carriers, stabilizers, preservatives and/or bulking agents, and is suitable for administration to a patient to achieve a desired diagnostic result or therapeutic effect. The pharmaceutical compositions disclosed herein have diagnostic, therapeutic, cosmetic, and/or research utility.

The anaerobic, gram positive bacterium Clostridium botulinum produces a potent polypeptide neurotoxin called botulinum neurotoxin toxin which causes a neuroparalytic illness in humans and animals referred to as botulism. Seven, generally immunologically distinct botulinum neurotoxins have been characterized, these being respectively botulinum neurotoxin serotypes A, B, C1, D, E, F and G each of which is distinguished by neutralization with type-specific antibodies. The different serotypes of botulinum toxin vary in the animal species that they affect and in the severity and duration of the paralysis they evoke. Botulinum toxin apparently binds with high affinity to cholinergic motor neurons, is translocated into the neuron and blocks the release of acetylcholine.

Botulinum toxins have been used for the treatment of various therapeutic and cosmetic conditions. A botulinum toxin type A (Allergan, Inc., BOTOX®) is commercially available for the treatment of blepharo spasm, strabismus, cervical dystonia, hyperhydrosis and glabellar lines. BOTOX® consists of a purified botulinum toxin type A complex, albumin and sodium chloride packaged in sterile, vacuum-dried form. Each vial of BOTOX® contains about 100 units (U) of Clostridium botulinum toxin type A purified neurotoxin complex, 0.5 milligrams of human serum albumin and 0.9 milligrams of sodium chloride in a sterile, vacuum-dried form without a preservative. Other commercially available botulinum neurotoxins approved for use in humans include DYSPORT® (Beaufour Ipsen, Porton Down, England), XEOMIN® (Merz Pharmaceuticals GmbH, Frankfurt, Germany), JEAUVEAU (Evolus, Newport Beach, Calif.), and MYOBLOC® (Solstice Neurosciences, San Francisco, Calif.).

The molecular weight of the neurotoxic component of a botulinum toxin complex is about 150 kD. Botulinum toxin is typically made by the Clostridial botulinum bacterium as a complex comprising the 150 kD botulinum toxin protein molecule and associated non-toxin proteins. Thus, a botulinum toxin type A complex can be produced by Clostridial bacterium as 900 kD, 500 kD and 300 kD complex forms.

Injection of a botulinum toxin into facial muscles can, by weakening the injected muscles, result in a decrease of hyperkinetic wrinkles in the skin overlying the paralyzed muscles (Carruthers, A. et al., J. Dermatol. Surg. Oncol., January 1990; 16(1):83). Botulinum toxin has been injected into facial muscles, such as the orbicularis oculis, corrugator supercilii and frontalis muscles for the cosmetic purpose of reducing certain facial wrinkles. A method to assess efficacy of injection is via electromyographic and/or photographic techniques (Guerrissi, J. et al., Ann Plast Surg, 1997; 39(5):447-53). Electromyography has also been used to assess the effect of injection of a botulinum toxin into the sternocleidomastoid muscle for treatment of cervical dystonia (Dressler, D. et al., Eur Neurol 2000; 43: 13-16). In this technique, surface electrodes are placed at fixed distances from the injection point, typically 1 cm and 3 cm from the injection point. The surface electrodes are used to measure the amplitude and area of a compound muscle action potential (CMAP) during maximal voluntary contraction of the injected muscle. Typically, CMAP decreases with the onset of muscle paralytic effect and to increase as the paralytic effect wears off.

Photographic methods, such as digital image analysis, have also been used to determine efficacy of a botulinum toxin to treat hyperkinetic facial lines (Heckmann M., et al., J Am Acad Dermatol 2001; 45: 508-514). The potency of any given botulinum toxin preparation can also be routinely assessed by using the Digital Abduction Score (DAS) assay, which measures the local muscle weakening efficacy of botulinum toxin following injection into mouse or rat hindlimb muscle (Broide, R. S. et al., J Toxicon, 2013, 71:18-24).

Formulations comprising a Clostridial toxin, such as a botulinum toxin, that offer an improved effect and/or duration of the effect are needed.

### BRIEF SUMMARY

In one aspect, a pharmaceutical composition comprising a Clostridial toxin active ingredient and a non-cross-linked hyaluronic acid or salt thereof, wherein the weight average molecular weight of a non-cross-linked hyaluronic acid or salt thereof is from 250 kilodaltons (kDa) to 2.4 megadaltons (MDa) or from 4.6 MDa to 8 MDa, is provided.

In another aspect, a pharmaceutical composition comprising a Clostridial toxin active ingredient, a non-cross-linked hyaluronic acid or salt thereof, a tonicity agent, a surfactant and an antioxidant is provided

In another aspect, a pharmaceutical composition comprising a Clostridial toxin active ingredient, a non-cross-linked hyaluronic acid or salt thereof, a surfactant and an antioxidant is provided.

In another aspect, a pharmaceutical composition comprising a Clostridial toxin active ingredient, a non-cross-linked hyaluronic acid or salt thereof, a lyoprotector, a surfactant and an antioxidant is provided.

In another aspect, a pharmaceutical composition comprising a non-cross-linked hyaluronic acid or salt thereof, a tonicity agent, a surfactant and an antioxidant is provided

In another aspect, a pharmaceutical composition comprising a non-cross-linked hyaluronic acid or salt thereof, a surfactant and an antioxidant is provided.

In another aspect, a pharmaceutical composition comprising a non-cross-linked hyaluronic acid or salt thereof, a lyoprotector, a surfactant and an antioxidant is provided.

In some embodiments, the pharmaceutical compositions comprise a botulinum toxin. In some embodiments, the pharmaceutical composition comprises trehalose. In some embodiments, the pharmaceutical composition comprises sodium chloride. In some embodiments, the composition comprises a poloxamer and/or a polysorbate. In some embodiments, the composition comprises poloxamer 188 and/or polysorbate 20. In some embodiments, the antioxidant is selected from the group consisting of L-methionine, N-acetyl-cysteine (NAC), butylated hydroxytoluene (BHT), ethylene diamine tetraacetic acid sodium salt (EDTA), an EDTA analog, ethylene glycol-bis(2-aminoethylether)-N,N,N′,N′-tetraacetic acid (EGTA), an EGTA analog, diethylenetriaminepentaacetic acid (DTPA), a DTPA analog, ascorbic acid, and combinations thereof. In some embodiments, the composition further comprises a buffering agent. In one embodiment, the buffering agent includes histidine buffer. In some embodiments, the composition has a pH of from 5 to 7. In some embodiments, the composition is a liquid formulation. In some embodiments, the composition is a solid lyophilized formulation.

In some embodiments, the composition comprises a first part and a second part, where the first part comprises a Clostridial toxin active ingredient and one or more pharmaceutically acceptable excipients, and the second part comprises a non-cross-linked hyaluronic acid or salt thereof dissolved or suspended in a buffer, where the first part and the second part are combinable to form a liquid composition. In one embodiment, the first part is a solid lyophilized composition. In another embodiment, the first part is a liquid formulation.

In another aspect, a liquid pharmaceutical composition comprising a Clostridial toxin active ingredient, a non-cross-linked hyaluronic acid or salt thereof, trehalose, a poloxamer or a polysorbate, and L-methionine or NAC is provided. In some embodiments, the liquid pharmaceutical composition comprises a botulinum toxin. In some embodiments, the liquid pharmaceutical composition further comprises EDTA, EGTA, DTPA or analogues thereof. In some embodiments, the liquid pharmaceutical composition comprises a histidine buffer. In some embodiments, the pH of the liquid pharmaceutical composition ranges from 5 to 7. In some embodiments, the relative weight amount of L-methionine ranges from about 0.1% to about 0.3%. In some embodiments, the relative weight amount of NAC ranges from about 0.1% to about 0.5%. In some embodiments, the relative weight amount of EDTA ranges from about 0.01% to about 0.05%. In some embodiments, the relative weight amount of trehalose ranges from about 1.0 to about 10%. In some embodiments, the relative weight amount of poloxamer 188 ranges from about 0.5% to about 5%. In some embodiments, the relative weight amount of polysorbate ranges from about 0.02% to about 0.06%.

In another embodiment, a liquid pharmaceutical composition is provided. The composition comprises a Clostridial toxin active ingredient; a non-cross-linked hyaluronic acid or salt thereof; a tonicity agent selected from trehalose, sucrose and combinations thereof; a surfactant selected from a poloxamer, a polysorbate and combinations thereof; and an antioxidant selected from methionine, NAC, ascorbic acid, butylated hydroxytoluene, EDTA, EGTA, DTPA, analogues thereof, and combinations thereof. In one embodiment, the composition excludes albumin, and in another embodiment, when the antioxidant is methionine the composition excludes a polysorbate. In one embodiment, the composition excludes animal protein stabilizers.

In another embodiment, a liquid pharmaceutical composition is contemplated. The composition comprises a Clostridial toxin active ingredient; a non-cross-linked hyaluronic acid or salt thereof; a surfactant selected from a poloxamer, a polysorbate and combinations thereof; and an antioxidant selected from methionine, NAC, EDTA, EGTA, DTPA, analogues thereof, and combinations thereof. In one embodiment, the composition excludes albumin.

In another embodiment, the liquid composition is an animal protein free composition that comprises a botulinum toxin, a non-cross-linked hyaluronic acid or salt thereof, a poloxamer, and methionine, and, optionally, includes a disaccharide. In one embodiment, the liquid composition excludes a disaccharide.

In a further embodiment, the liquid composition is an animal protein free composition that comprises a botulinum toxin, a non-cross-linked hyaluronic acid or salt thereof, a surfactant selected from a poloxamer, a polysorbate and combinations thereof; and an antioxidant selected from the group consisting of a chelating agent, a sacrificial antioxidant, a chain terminator, and combinations thereof. In one embodiment, the antioxidant includes a combination of a chelating agent and a chain terminator.

In yet another embodiment, the liquid composition is an animal-protein free composition comprising a botulinum toxin, a non-cross-linked hyaluronic acid or salt thereof, a poloxamer surfactant, and methionine, and, optionally, a disaccharide. In one embodiment, the liquid composition excludes a disaccharide.

In another embodiment, a liquid pharmaceutical composition is contemplated. The composition comprises a Clostridial toxin active ingredient; a non-cross-linked hyaluronic acid or salt thereof; trehalose or sucrose; a poloxamer; and methionine. In one embodiment, the composition excludes albumin.

In another embodiment, a liquid pharmaceutical composition is contemplated. The composition comprises a Clostridial toxin active ingredient; a non-cross-linked hyaluronic acid or salt thereof; trehalose in an amount between 1-15 wt %; a poloxamer in an amount between 0.5-8 wt %; and methionine in an amount between 0.05-5 wt %. In one embodiment, the composition excludes albumin.

In another embodiment, a liquid pharmaceutical composition is contemplated. The composition comprises a botulinum toxin; a non-cross-linked hyaluronic acid or salt thereof; a disaccharide; a poloxamer; and an antioxidant selected from methionine, NAC, ascorbic acid, butylated hydroxytoluene, EDTA, EGTA, DTPA, analogues thereof, and combinations thereof. In one embodiment, the composition excludes albumin.

In another embodiment, the liquid composition is an animal protein free composition that comprises a botulinum toxin; a non-cross-linked hyaluronic acid or salt thereof; a disaccharide; a poloxamer; and an antioxidant selected from the group consisting of a chelating agent, a sacrificial antioxidant, a chain terminator, and combinations thereof.

In another embodiment, a liquid composition comprised of a Clostridial toxin active ingredient, a non-cross-linked hyaluronic acid or salt thereof, a poloxamer, a chelating agent and a chain terminator is provided.

In another embodiment, a liquid composition comprised of a Clostridial toxin active ingredient; a non-cross-linked hyaluronic acid or salt thereof; a poloxamer; a chelating agent selected from EDTA, EGTA, DTPA and analogues thereof; and NAC is provided.

In another embodiment, a liquid composition comprised of a Clostridial toxin active ingredient, a non-cross-linked hyaluronic acid or salt thereof, a poloxamer, and methionine is provided.

In another embodiment, a liquid composition comprised of a Clostridial toxin active ingredient, a non-cross-linked hyaluronic acid or salt thereof, a tonicity agent selected from trehalose, sucrose and combinations thereof, a surfactant selected from a poloxamer, a polysorbate and combinations thereof, a chelating agent and a chain terminator is provided.

In another embodiment, a liquid composition comprised of a Clostridial toxin active ingredient; a non-cross-linked hyaluronic acid or salt thereof; a tonicity agent selected from trehalose, sucrose and combinations thereof; a surfactant selected from a poloxamer, a polysorbate and combinations thereof; a chelating agent selected from EDTA, EGTA, DTPA and analogues thereof; and NAC, is provided.

In any of the foregoing embodiments, it is contemplated that the composition is not, in some embodiments, an emulsion and/or excludes nanoparticles comprising an amphiphilic entity.

In another aspect, the present disclosure provides a solid pharmaceutical composition comprising a botulinum toxin; a non-cross-linked hyaluronic acid or salt thereof; trehalose; a poloxamer or a polysorbate; NAC; and a chelating agent selected from EDTA, EGTA, DTPA and analogues thereof. In an alternative embodiment, the solid pharmaceutical composition comprises a botulinum toxin, a non-cross-linked hyaluronic acid or salt thereof, trehalose, a poloxamer and L-methionine. In some embodiments, the solid pharmaceutical composition further comprises histidine buffer. In some embodiments, the relative weight amount of L-methionine ranges from about 0.1% to about 0.3%. In some embodiments, the relative weight amount of NAC ranges from about 0.01% to about 0.5%. In some embodiments, the relative weight amount of EDTA ranges from about 0.01% to about 0.05%. In some embodiments, the relative weight amount of trehalose ranges from about 1.0 to about 10%. In some embodiments, the relative weight amount of poloxamer ranges from about 0.5% to about 5%. In some embodiments, the relative weight amount of polysorbate ranges from about 0.02% to about 0.06%.

In one embodiment, a solid or lyophilized pharmaceutical composition is contemplated. The composition comprises a Clostridial toxin active ingredient; a non-cross-linked hyaluronic acid or salt thereof; a disaccharide selected from trehalose, sucrose and combinations thereof; a surfactant selected from a poloxamer, a polysorbate and combinations thereof; and an antioxidant selected from methionine, N-acetyl cysteine, BHT, EDTA, EGTA, DTPA, ascorbic acid, analogues thereof, and combinations thereof. In one embodiment, the composition excludes albumin, a hydroxyalkylstarch, glutamic acid, glutamine, aspartic acid, asparagine, a polyalcohol, glycine, and/or polyvinylpyrrolidone.

In another embodiment, the solid or lyophilized composition is an animal protein free composition that comprises a botulinum toxin; a non-cross-linked hyaluronic acid or salt thereof; a disaccharide selected from trehalose, sucrose and combinations thereof; a surfactant selected from a poloxamer, a polysorbate and combinations thereof; and an antioxidant selected from the group consisting of a chelating agent, a sacrificial antioxidant, a chain terminator, and combinations thereof.

In another embodiment, a solid or lyophilized pharmaceutical composition is contemplated. The composition comprises a Clostridial toxin active ingredient; a non-cross-linked hyaluronic acid or salt thereof; trehalose or sucrose; a poloxamer; and methionine. In one embodiment, the composition excludes albumin, a hydroxyalkylstarch, glutamic acid, glutamine, aspartic acid, asparagine, a polyalcohol, glycine, and/or polyvinylpyrrolidone.

In another embodiment, a solid or lyophilized pharmaceutical composition is contemplated. The composition comprises a Clostridial toxin active ingredient; a non-cross-linked hyaluronic acid or salt thereof; trehalose in an amount between 1-15 wt %; a poloxamer in an amount between 0.5-8 wt %; and methionine in an amount between 0.05-5 wt %. In one embodiment, the composition excludes albumin, a hydroxyalkylstarch, glutamic acid, glutamine, aspartic acid, asparagine, a polyalcohol, glycine, and/or polyvinylpyrrolidone.

In another embodiment, a solid or lyophilized pharmaceutical composition is contemplated. The composition comprises a botulinum toxin; a non-cross-linked hyaluronic acid or salt thereof; a disaccharide; a poloxamer; and an antioxidant selected from methionine, N-acetyl cysteine, EDTA, EGTA, DTPA, analogues thereof, and combinations thereof. In one embodiment, the composition excludes albumin, a hydroxyalkylstarch, glutamic acid, glutamine, aspartic acid, asparagine, a polyalcohol, glycine, and/or polyvinylpyrrolidone.

In another embodiment, the solid or lyophilized composition is an animal protein free composition that comprises a botulinum toxin; a non-cross-linked hyaluronic acid or salt thereof; a disaccharide; a poloxamer; and an antioxidant selected from the group consisting of a chelating agent, a sacrificial antioxidant, a chain terminator, and combinations thereof.

In another embodiment, a lyophilized composition comprised of a Clostridial toxin active ingredient; a non-cross-linked hyaluronic acid or salt thereof; a lyoprotector selected from sucrose, trehalose, mannitol, sorbitol, glucose, and combinations thereof; a surfactant selected from a poloxamer, a polysorbate and combinations thereof; a chelating agent; and a chain terminator; is provided.

In another embodiment, a lyophilized composition comprised of a Clostridial toxin active ingredient; a non-cross-linked hyaluronic acid or salt thereof; a lyoprotector selected from sucrose, trehalose, mannitol, sorbitol, glucose, and combinations thereof; a surfactant selected from a poloxamer, a polysorbate and combinations thereof; a chelating agent selected from EDTA, EGTA, DTPA, and analogs thereof; and NAC; is provided.

In another embodiment, a lyophilized composition comprised of a Clostridial toxin active ingredient; a non-cross-linked hyaluronic acid or salt thereof; a lyoprotector selected from sucrose, trehalose, mannitol, sorbitol, glucose, and combinations thereof; a poloxamer; and a chain terminator is provided. In one embodiment, the lyophilized composition excludes a chelating agent. In one embodiment, the chain terminator is NAC.

In another embodiment, a lyophilized composition comprised of a Clostridial toxin active ingredient; a non-cross-linked hyaluronic acid or salt thereof; a lyoprotector selected from sucrose, trehalose, mannitol, sorbitol, glucose, and combinations thereof; a poloxamer; and methionine is provided.

In another embodiment, a lyophilized composition comprised of a Clostridial toxin active ingredient; a non-cross-linked hyaluronic acid or salt thereof; a lyoprotector selected from sucrose, trehalose, mannitol, sorbitol, glucose, and combinations thereof; a poloxamer; a chelating agent; and a chain terminator is provided.

In another embodiment, a lyophilized composition comprised of a Clostridial toxin active ingredient; a non-cross-linked hyaluronic acid or salt thereof; a lyoprotector selected from sucrose, trehalose, mannitol, sorbitol, glucose, and combinations thereof; a poloxamer; a chelating agent selected from EDTA, EGTA, DTPA and analogues thereof; and NAC is provided.

In another embodiment, the lyophilized composition is an animal protein free composition that comprises a Clostridial toxin active ingredient; a non-cross-linked hyaluronic acid or salt thereof; a lyoprotector selected from sucrose, trehalose, mannitol, sorbitol, glucose, and combinations thereof; a poloxamer; and NAC; and optionally includes EDTA, EGTA, DTPA or analogues thereof. In one embodiment, the lyophilized composition excludes EDTA, EGTA, DTPA and analogues thereof.

In another embodiment, a lyophilized composition comprised of a Clostridial toxin active ingredient; a non-cross-linked hyaluronic acid or salt thereof; a lyoprotector selected from sucrose, trehalose, mannitol, sorbitol, glucose, and combinations thereof; a surfactant selected from a poloxamer, a polysorbate and combinations thereof; and NAC is provided.

In certain embodiments, the lyophilized composition is reconstituted with a tonicity agent selected from trehalose, sucrose, sodium chloride, mannitol, sorbitol, glucose, and combinations thereof. In at least one embodiment, the lyophilized composition is reconstituted with a reconstitution vehicle comprising NaCl prior to administration to a patient.

In any of the foregoing embodiments of solid or liquid compositions, it is contemplated that one or more, in any combination, of these ingredients are excluded polyvinylpyrrolidone, diblock copolymers of polypropylene glycol and polyethylene glycol, and/or a polyalcohol such as inositol, lactilol, isomalt, xylitol, erythritol. In any of the foregoing embodiments of solid or liquid compositions, it is contemplated that the composition is free of animal proteins.

In another aspect, a pharmaceutical composition comprising a Clostridial toxin active ingredient; a non-cross-linked hyaluronic acid or salt thereof; a tonicity agent and/or a lyoprotector selected from trehalose, sucrose and combinations thereof; a surfactant selected from a poloxamer, a polysorbate and combinations thereof; and an antioxidant selected from methionine, N-acetyl cysteine, EDTA, EGTA, DTPA, analogues thereof, and combinations thereof, is contemplated. In one embodiment, the composition excludes albumin, and in embodiments when the composition is a liquid and the antioxidant is methionine, the surfactant excludes a polysorbate. The composition can be liquid or solid.

In another aspect, a pharmaceutical composition is contemplated. The composition comprises a Clostridial toxin active ingredient; a non-cross-linked hyaluronic acid or salt thereof; trehalose or sucrose; a poloxamer; and methionine. In one embodiment, the composition excludes albumin. The composition can be liquid or solid.

In another aspect, a pharmaceutical composition is contemplated. The composition comprises a Clostridial toxin active ingredient, a non-cross-linked hyaluronic acid or salt thereof, trehalose in an amount between 1-15 wt %, a poloxamer in an amount between 0.5-8 wt %, and methionine in an amount between 0.05-5 wt %. In one embodiment, the composition excludes albumin. The composition can be liquid or solid.

In another aspect, a pharmaceutical composition is contemplated. The composition comprises a botulinum toxin; a non-cross-linked hyaluronic acid or salt thereof, a disaccharide; a poloxamer; and an antioxidant selected from methionine, NAC, EDTA, EGTA, DTPA, analogues thereof, and combinations thereof. In one embodiment, the composition excludes albumin. The composition can be liquid or solid.

In another aspect, a pharmaceutical composition comprising a Clostridial toxin active ingredient; a non-cross-linked hyaluronic acid or salt thereof; a surfactant selected from a poloxamer, a polysorbate and combinations thereof; and an antioxidant selected from methionine, N-acetyl cysteine, EDTA, EGTA, DTPA, analogues thereof, and combinations thereof, is provided. In one embodiment, the composition excludes albumin, and in embodiments when the composition is a liquid and the antioxidant is methionine, the surfactant excludes a polysorbate. The composition can be liquid or solid. In one embodiment, the composition further comprises a tonicity agent and/or a lyoprotector. In some embodiments, the tonicity agent is selected from trehalose, sucrose, sodium chloride, mannitol, sorbitol, glucose, and combinations thereof. In alternative embodiments, the lyoprotector is selected from trehalose, sucrose, mannitol, sorbitol, glucose, and combinations thereof. In one embodiment, the tonicity agent and/or lyoprotector is a disaccharide. In one embodiment, the disaccharide is selected from trehalose and sucrose.

In any of the foregoing aspects/embodiments of solid or liquid compositions, it is contemplated that the compositions do not comprise cross-linked hyaluronic acid.

In any of the foregoing aspects/embodiments of solid or liquid compositions, it is contemplated that the composition can be prepared by mixing a composition comprising Clostridial toxin active ingredient with a composition comprising a non-cross-linked hyaluronic acid or salt thereof and a diluent that is not a non-cross-linked hyaluronic acid or salt thereof, or by mixing a non-cross-linked hyaluronic acid or salt thereof with a composition comprising Clostridial toxin active ingredient and a diluent.

In any of the foregoing aspects/embodiments of liquid compositions, it is contemplated that the concentration of Clostridial toxin active ingredient is from 0.2 to 2.0 ng/mL or from about 0.2-10 ng/mL.

In any of the foregoing aspects/embodiments of solid or liquid compositions, it is contemplated that the concentration of Clostridial toxin active ingredient is from about 10 U/mL to about 200 U/mL or from about 10 U/mL to about 100 U/mL.

In any of the foregoing aspects/embodiments of solid or liquid compositions, it is contemplated that the composition comprises up to 10 units of Clostridial toxin active ingredient per milligram of a non-cross-linked hyaluronic acid or salt thereof. In another embodiment of the solid or liquid compositions, it is contemplated that the composition comprises up to 40 units of Clostridial toxin active ingredient per milligram of a non-cross-linked hyaluronic acid or salt thereof.

In any of the foregoing aspects/embodiments of solid or liquid compositions, it is contemplated that the Clostridial toxin active ingredient is botulinum toxin type A or Onabotulinum toxin A.

In any of the foregoing aspects/embodiments of solid or liquid compositions, it is contemplated that the compositions may not comprise any Clostridial toxin active ingredient.

In any of the foregoing aspects/embodiments of solid or liquid compositions, it is contemplated that the weight average molecular weight of a non-cross-linked hyaluronic acid is from about 450 kDa to 2.0 MDa, such as from 450 kDa to 1.6 MDa.

In any of the foregoing aspects/embodiments of solid or liquid compositions, it is contemplated that the weight average molecular weight of a non-cross-linked hyaluronic acid is about 1.58 MDa.

In any of the foregoing aspects/embodiments of solid or liquid compositions, it is contemplated that the concentration of a non-cross-linked hyaluronic acid is from 0.1 to 50 wt % based on the total weight of the solid or liquid compositions, such as from about 0.2 to 10 wt % or from about 0.4 to 5 wt %, such as about 0.4, 0.5, 0.6, 0.7, 0.8, 1.0, 1.1, 1.2, 1.3, 1.4, 1.5, 2, 2.5, 3, 3.5, 4, 4.5, 5, 5.5, or 6 wt %.

In any of the foregoing aspects/embodiments of solid or liquid compositions, the pharmaceutical composition can increases the efficacy and/or duration time of Clostridial toxin active ingredient by at least 10%, 20%, 30%, 40%, 50%, 60%, 70%, 80%, 90%, and 100% as compared to a pharmaceutical composition which does not comprise a non-cross-linked hyaluronic acid or salt thereof. In some particular embodiments of such pharmaceutical compositions, a non-cross-linked hyaluronic acid or salt thereof is present in an amount of about 1.2 wt % and has the weight average molecular weight of about 1.58 Mda.

In any of the foregoing aspects/embodiments of solid or liquid compositions, when the composition comprises botulinum toxin type A, about 1.2 wt % of a non-cross-linked hyaluronic acid or salt thereof which has a weight average molecular weight of about 1.58 MDa, the efficacy and/or duration of effect of botulinum toxin type A can be about twice as much as that of a composition which does not comprise any non-cross-linked hyaluronic acid or salt thereof.

In any of the foregoing aspects/embodiments of liquid compositions, it is contemplated that the viscosity of the pharmaceutical composition is from about 0.01 Pa-S to about 0.2 Pa-s (about 10 cps to about 200 cps) at 25° C., at a shear rate of 0.1/second. In one embodiment, the composition has a viscosity of between about 5-500 Pa-s at a shear rate of 0.1/sec at 25° C. In another embodiment, the composition has a viscosity of between about 50-250 Pa-s at a shear rate of 0.1/sec at 25° C.

In another aspect, a method for treating depression is contemplated. The method comprises providing for administration, instructing to administer, or administering a composition according to any of the embodiments and aspects described herein.

In another aspect, a method for treating cardiac arrhythmia is contemplated. The method comprises providing for administration, instructing to administer, or administering a composition according to any of the embodiments and aspects described herein.

In another aspect, a method for treating glabellar lines is contemplated. The method comprises providing for administration, instructing to administer, or administering a composition according to any of the embodiments and aspects described herein.

In another aspect, a method for cervical dystonia is contemplated. The method comprises providing for administration, instructing to administer, or administering a composition according to any of the embodiments and aspects described herein. In one embodiment, the method is effective to reduce the severity of abnormal head position and neck pain.

In another aspect, a method for lateral canthal lines is contemplated. The method comprises providing for administration, instructing to administer, or administering a composition according to any of the embodiments and aspects described herein.

In another aspect, a method for forehead lines is contemplated. The method comprises providing for administration, instructing to administer, or administering a composition according to any of the embodiments and aspects described herein.

In another aspect, a method for treating overactive bladder with symptoms of urge urinary incontinence, urgency, and frequency, in adults who have an inadequate response to or are intolerant of an anticholinergic medication is contemplated. The method comprises providing for administration, instructing to administer, or administering a composition according to any of the embodiments and aspects described herein.

In another aspect, a method for treating urinary incontinence due to detrusor over activity associated with a neurologic condition (e.g., spinal cord injury (SCI), multiple sclerosis (MS)) in adults who have an inadequate response to or are intolerant of an anticholinergic medication is contemplated. The method comprises providing for administration, instructing to administer, or administering a composition according to any of the embodiments and aspects described herein.

In another aspect, a method for treating or for the prophylactic treatment of headaches in adult patients with chronic migraine (>15 days per month with headache lasting 4 hours a day or longer) is contemplated. The method comprises providing for administration, instructing to administer, or administering a composition according to any of the embodiments and aspects described herein.

In another aspect, a method for treating upper and/or lower limb spasticity in adult patients and in pediatric patients (ages 2-17) is contemplated. The method comprises providing for administration, instructing to administer, or administering a composition according to any of the embodiments and aspects described herein.

In another aspect, a method for treating axillary hyperhidrosis is contemplated. The method comprises providing for administration, instructing to administer, or administering a composition according to any of the embodiments and aspects described herein. In one embodiment, the method is intended for human subjects where the axillary hyperhidrosis is severe and/or is inadequately managed by topical agents.

In another aspect, a method for treating strabismus is contemplated. The method comprises providing for administration, instructing to administer, or administering a composition according to any of the embodiments and aspects described herein. In an embodiment, the strabismus is in a human patient 12 years of age and older.

### BRIEF DESCRIPTION OF THE FIGURES

FIG. 1A is a graph of mean DAS as a function of time, in days, following injection of compositions with botulinum toxin serotype A (BoNT/A) into rats; the compositions lacking non-cross-linked hyaluronic acid (controls) given at BoNT/A doses of 4.7 U/kg (open squares), 9.4 U/kg (open circles), and 16.4 U/kg (open triangles), and the compositions with 1.2 wt % linear, non-cross-linked hyaluronic acid (weight average MW of 1500 kDa) given at BoNT/A doses of 4.7 U/kg (closed squares) and 9.4 U/kg (closed circles);

FIG. 1B is a graph of average DAS as a function of time, in days, following injection of compositions with BoNT/A into rats of compositions with BoNT/A at a dose of 2.96 U/kg, with one composition (closed diamonds) comprising 1.2 wt % of a linear, non-cross-linked hyaluronic acid (weight average MW of 1500 kDa), and the control composition (open diamonds) with no linear, non-cross-linked hyaluronic acid;

FIG. 1C is a graph of mean DAS as a function of time, in days, following injection of compositions with BoNT/A into rats; all compositions except one were given at BoNT/A doses of 4.7 U/kg, and one control, comparator composition dosed at 9.4 U/kg (open circles); the test compositions comprised 1.2% non-cross-linked hyaluronic acid (1500 kDa; closed squares), 2% non-cross-linked hyaluronic acid (1500 kDa, closed diamonds), 3% non-cross-linked hyaluronic acid (LMW, closed circles), and 0.6% non-cross-linked hyaluronic acid (HHMW, closed inverted triangles); the comparator, control compositions lacked non-cross-linked hyaluronic acid and were dosed at 4.7 U/kg BoNT/A (open squares) or at 9.4 U/kg (open circles);

FIG. 2 is a graph of mean DAS as a function of time, in days, following injection of compositions with BoNT/A into rats, the compositions 1.2 wt % of a non-cross-linked hyaluronic acid (closed squares) or no non-cross-linked-hyaluronic acid (open squares);

FIG. 3 is a graph of mean DAS as a function of time, in days, following injection into rats of botulinum toxin control compositions lacking a non-cross-linked hyaluronic acid and administered with BoNT/A doses of 9 U/kg (open circles), 5.1 U/kg (open squares), or 2.8 U/kg (open triangles) or with compositions comprising 1.2 wt % non-cross-linked hyaluronic acid (1500 kDa) administered at BoNT/A doses of 9 U/kg (closed circles), 5.1 U/kg (closed squares), or 2.8 U/kg (closed triangles);

FIG. 4 is a graph of mean DAS as a function of time, in days, following injection into rats of botulinum toxin control compositions lacking a non-cross-linked hyaluronic acid and administered with BoNT/A doses of 2.85 U/kg (open triangles) or 9 U/kg (open circles) or with compositions comprising 1.2 wt % non-cross-linked hyaluronic acid (1500 kDa) administered at BoNT/A doses of 2.85 U/kg (closed triangles) or 9 U/kg (closed circles);

FIG. 5 is a graph of mean DAS as a function of time, in days, following injection into rats of botulinum toxin control compositions lacking a non-cross-linked hyaluronic acid and administered with BoNT/A doses of 2.96 U/kg (open triangles), 4.70 U/kg (open squares), or 9.4 U/kg (open circles) or with compositions comprising 1.2 wt % non-cross-linked hyaluronic acid (1500 kDa) administered at BoNT/A doses of 2.96 U/kg (closed triangles), 4.70 U/kg (closed squares), or 9.4 U/kg (closed circles);

FIG. 6 is a graph of average DAS as a function of time, in days, following injection into rats of botulinum toxin compositions, dosed at 9.4 U/kg, the botulinum toxin in compositions with histidine, trehalose, surfactant (Poloxamer P188) and methionine, pH 6, with no non-cross-linked hyaluronic acid (control, open squares) or with 1.2% non-cross-linked hyaluronic acid with 1500 kDa average MW (closed circles) or 2.3% non-crosslinked hyaluronic acid with 700 kDa average MW (closed triangles);

FIG. 7 is a graph of average DAS as a function of time, in days, following injection into rats of botulinum toxin compositions, dosed at 9.4 U/kg, the botulinum toxin in compositions with histidine, trehalose, surfactant (Poloxamer P188) and methionine, pH 6, with no non-cross-linked hyaluronic acid (control, open squares) or non-cross-linked hyaluronic acid with 1500 kDa average MW at 0.6 wt % (open circles), 1.2 wt % (open triangles), 1.6 wt % (closed circles), and 2.0 wt % (closed triangles); and

FIG. 8 is a graph of average DAS as a function of time, in days, following injection into rats of botulinum toxin compositions, dosed at 9.4 U/kg, the botulinum toxin compositions comprising 1.2 wt % non-cross-linked hyaluronic acid with 1500 kDa average MW in various vehicles identified in Table A below where the formulation number assigned in the table corresponds to the symbols as follows: Formulation 1 (control, no non-cross-linked hyaluronic acid), open squares; Formulation 2, open triangles; Formulation 3, closed squares; Formulation 4, closed circles; Formulation 5, closed triangles; Formulation 6, inverted triangles; Formulation 7, closed diamonds.

### DETAILED DESCRIPTION

Compositions described herein, in embodiments, are directed to stable liquid and/or stable solid pharmaceutical compositions of a Clostridial toxin active ingredient and/or a non-cross-linked hyaluronic acid or salt thereof. In some embodiments, the composition further comprises one or more of a surfactant and an antioxidant, and, optionally, a tonicity agent and/or a lyoprotector. In certain liquid compositions, a lyoprotector in the form of a disaccharide is optional.

Also as will be described below, the compositions are useful in methods for the treatment of various diseases, disorders, and conditions, including, for example, depression (e.g. major depressive disorder), headache (e.g. migraine, tension headache, and the like), pain, atrial fibrillation, hyperhidrosis, muscle spasticity, cervical dystonia, blepherospasm, overactive bladder (e.g. neurogenic detrusor over-activity, and idiopathic overactive bladder), bladder pain (e.g. interstitial cystitis, and bladder pain syndrome), skin conditions (e.g. wrinkles, fine wrinkles, excess sebum production, acne, and rosacea), irregularities, and the like using the compositions provided herein. Embodiments can include various administration techniques, including, for example, injection, such as intramuscular, intracutaneous, subcutaneous, or the like, instillation, intravenous, transdermal, and topical.

### Definitions

As used herein, the words or terms set forth below have the following definitions:

The articles “a” and “an” are used herein to refer to one or to more than one (i.e., to at least one) of the grammatical object of the article. By way of example, “an element” means one element or more than one element.

“About” or “approximately” as used herein means within an acceptable error range for the particular value as determined by one of ordinary skill in the art, which will depend in part on how the value is measured or determined, (i.e., the limitations of the measurement system). For example, “about” can mean within 1 or more than 1 standard deviations, per practice in the art. Where particular values are described in the application and claims, unless otherwise stated, the term “about” means within an acceptable error range for the particular value. The term “about” when qualifying a value of a stated item, number, percentage, or term refers to a range of plus or minus ten percent of the value of the stated item, percentage, parameter, or term.

“Administration” or “to administer” means the step of giving (i.e. administering) a pharmaceutical composition to a subject, or alternatively a subject receiving a pharmaceutical composition. The pharmaceutical compositions disclosed herein can be locally administered by various methods. For example, intramuscular, intradermal, subcutaneous administration, intrathecal administration, intraperitoneal administration, topical (transdermal), instillation, and implantation (for example, of a slow-release device such as polymeric implant or miniosmotic pump) can all be appropriate routes of administration.

“Alleviating” means a reduction in the occurrence of a pain, of a headache, or of any symptom or cause of a condition or disorder. Thus, alleviating includes some reduction, significant reduction, near total reduction, and total reduction.

“Animal protein free” means the absence of blood derived, blood pooled and other animal derived products or compounds. “Animal” means a mammal (such as a human), bird, reptile, fish, insect, spider or other animal species. “Animal” excludes microorganisms, such as bacteria. Thus, an animal protein free pharmaceutical composition can include a botulinum neurotoxin. For example, an “animal protein free” pharmaceutical composition means a pharmaceutical composition which is either substantially free or essentially free or entirely free of a serum derived albumin, gelatin and other animal derived proteins, such as immunoglobulins. An example of an animal protein free pharmaceutical composition is a pharmaceutical composition which comprises or which consists of a botulinum toxin (as the active ingredient) and a suitable polysaccharide as a stabilizer or excipient.

“Antioxidant” refers to any compound which protects an active ingredient from reaction with oxygen. Antioxidants can be broadly divided into three categories: (i) sacrificial antioxidants, which react with oxygen more readily than a particular active ingredient and therefore can scavenge oxygen, e.g., ascorbic acid and sulfites; (ii) chain terminators, which are molecules that form stable radicals due to weak bonds to hydrogen atoms that are attacked in a propagation of radical chains by consumption of oxygen, e.g., methionine, NAC, glutathionine, lipoic acid, butylated hydroxytoluene (BHT), and cysteine, (iii) chelating agents, which reduce catalytic activity of transition metals by forming complexes with the metals, e.g., EDTA, EGTA and DTPA and analogues thereof.

“Biological activity” describes the beneficial or adverse effects of a drug on living matter. When a drug is a complex chemical mixture, this activity is exerted by the substance's active ingredient but can be modified by the other constituents. Biological activity can be assessed as potency or as toxicity by an in vivo LD₅₀ or ED₅₀ assay, or through an in vitro assay such as, for example, cell-based potency assays as described in U.S. 2010/0203559 and U.S. 2010/0233802, incorporated by reference herein.

“Botulinum toxin” means a neurotoxin produced by Clostridium botulinum, as well as a botulinum toxin (or the light chain or the heavy chain thereof) made recombinantly by a non-Clostridial species. The phrase “botulinum toxin”, as used herein, encompasses the botulinum toxin serotypes A, B, C, D, E, F and G, and their subtypes and any other types of subtypes thereof, or any re-engineered proteins, analogs, derivatives, homologs, parts, sub-parts, variants, or versions, in each case, of any of the foregoing. “Botulinum toxin”, as used herein, also encompasses a “modified botulinum toxin”. Further “botulinum toxin” as used herein also encompasses a botulinum toxin complex, (for example, the 300, 600 and 900 kDa complexes), as well as the neurotoxic component of the botulinum toxin (150 kDa) that is unassociated with the complex proteins.

“Clostridial toxin” refers to any toxin produced by a Clostridial toxin strain that can execute the overall cellular mechanism whereby a Clostridial toxin intoxicates a cell and encompasses the binding of a Clostridial toxin to a low or high affinity Clostridial toxin receptor, the internalization of the toxin/receptor complex, the translocation of the Clostridial toxin light chain into the cytoplasm and the enzymatic modification of a Clostridial toxin substrate. Non-limiting examples of Clostridial toxins include Botulinum toxins, such as a BoNT/A (i.e. botulinum toxin serotype A), a BoNT/B, a BoNT/C₁, a BoNT/D, a BoNT/E, a BoNT/F, a BoNT/G, a Tetanus toxin (TeNT), a Baratii toxin (BaNT), and a Butyricum toxin (BuNT). The BoNT/C₂ cytotoxin and BoNT/C₃ cytotoxin, not being neurotoxins, are excluded from the term “Clostridial toxin.” The term Clostridial toxin also includes the approximately 150-kDa Clostridial toxin alone (i.e. without the NAPs). A Clostridial toxin includes naturally occurring Clostridial toxin variants, such as, e.g., Clostridial toxin isoforms and Clostridial toxin subtypes; non-naturally occurring Clostridial toxin variants, such as, e.g., conservative Clostridial toxin variants, non-conservative Clostridial toxin variants, Clostridial toxin chimeric variants and active Clostridial toxin fragments thereof, or any combination thereof. A Clostridial toxin also includes Clostridial toxin complexes, which refers to a complex comprising a Clostridial toxin and non-toxin associated proteins (NAPs), such as, e.g., a Botulinum toxin complex, a Tetanus toxin complex, a Baratii toxin complex, and a Butyricum toxin complex. Non-limiting examples of Clostridial toxin complexes include those produced by a Clostridium botulinum, such as, e.g., a 900-kDa BoNT/A complex, a 500-kDa BoNT/A complex, a 300-kDa BoNT/A complex, a 500-kDa BoNT/B complex, a 500-kDa BoNT/C₁ complex, a 500-kDa BoNT/D complex, a 300-kDa BoNT/D complex, a 300-kDa BoNT/E complex, and a 300-kDa BoNT/F complex.

“Clostridial toxin active ingredient” refers to a molecule which contains any part of a Clostridial toxin that exerts an effect upon or after administration to a subject or patient. As used herein, the term “Clostridial toxin” encompasses (i) a Clostridial toxin complex comprising the approximately 150-kDa Clostridial toxin and other proteins collectively called non-toxin associated proteins (NAPs), (ii) the approximately 150-kDa Clostridial toxin alone (i.e. without the NAPs), or (iii) a modified Clostridial toxin, such as, e.g., a re-targeted Clostridial toxins.

“Deformity” means a cosmetic, physical or functional irregularity, defect, abnormality, imperfection, malformation, depression, or distortion.

“Diluent” means components other than the Clostridial toxin active ingredient in the pharmaceutical compositions.

“Effective amount” as applied to the biologically active ingredient means that amount of the ingredient which is generally sufficient to effect a desired change in the subject. For example, where the desired effect is a reduction in an autoimmune disorder symptom, an effective amount of the ingredient is that amount which causes at least a substantial reduction of the autoimmune disorder symptom, and without resulting in significant toxicity.

“Effective amount” when used in reference to the amount of an excipient or specific combination of excipients added to a Clostridial toxin composition, refers to the amount of each excipient that is necessary to achieve the desired initial recovered potency of a Clostridial toxin active ingredient. In aspects of this embodiment, an effective amount of an excipient or combination of excipients results in an initial recovered potency of, e.g., at least 10%, at least 20%, at least 30%, at least 40%, at least 50%, at least 60%, at least 70%, at least 80%, at least 90% or at least 100%. In other aspects of this embodiment, a therapeutically effective concentration of a Clostridial toxin active ingredient reduces a symptom associated with the aliment being treated by, e.g., at most 10%, at most 20%, at most 30%, at most 40%, at most 50%, at most 60%, at most 70%, at most 80%, at most 90% or at most 100%.

“Heavy chain” means the heavy chain of a botulinum neurotoxin. It has a molecular weight of about 100 kDa and can be referred to as the H chain, or as H.

H_{C} means a fragment (about 50 kDa) derived from the H chain of a botulinum neurotoxin which is approximately equivalent to the carboxyl end segment of the H chain, or the portion corresponding to that fragment in the intact H chain. It is believed to be immunogenic and to contain the portion of the natural or wild type botulinum neurotoxin involved in high affinity, presynaptic binding to motor neurons.

H_{N} means a fragment (about 50 kDa) derived from the H chain of a botulinum neurotoxin which is approximately equivalent to the amino end segment of the H chain, or the portion corresponding to that fragment in the intact in the H chain. It is believed to contain the portion of the natural or wild type botulinum neurotoxin involved in the translocation of the L chain across an intracellular endosomal membrane.

“Light chain” means the light chain of a Clostridial neurotoxin. It has a molecular weight of about 50 kDa, and can be referred to as the L chain, L, or as the proteolytic domain (amino acid sequence) of a botulinum neurotoxin.

LH_{N} or L-H_{N} means a fragment derived from a Clostridial neurotoxin that contains the L chain, or a functional fragment thereof coupled to the H_{N} domain It can be obtained from the intact Clostridial neurotoxin by proteolysis, so as to remove or to modify the H_{C} domain.

“Implant” means a controlled release (e.g., pulsatile or continuous) composition or drug delivery system. The implant can be, for example, injected, inserted or implanted into a human body.

“Liquid composition”, “liquid pharmaceutical composition”, or “liquid formulation” refers to a pharmaceutically active preparation of drug or biological which is capable of being stored in a liquid pharmaceutical excipient, such as a buffering agent, for an extended period of time, such that it can be ready-to-use as needed by a clinician. The liquid pharmaceutical composition is manufactured without a lyophilization process.

“Local administration” means direct administration of a pharmaceutical at or to the vicinity of a site on or within an animal body, at which site a biological effect of the pharmaceutical is desired, such as via, for example, intramuscular or intra- or subdermal injection or topical administration. Local administration excludes systemic routes of administration, such as intravenous or oral administration. Topical administration is a type of local administration in which a pharmaceutical agent is applied to a patient's skin.

“Lyoprotector” or “lyoprotectant” means a substance that is included in a lyophilized formulation to protect a Clostridial toxin active ingredient during the freeze-drying process. Lyoprotectors include for example polyhydroxy compounds such as sugars (mono-, di-, and polysaccharides), polyalcohols, and their derivatives. Exemplary lyoprotectors which can be used with the lyophilized formulations disclosed herein include sucrose, trehalose, mannitol, sorbitol, glucose, raffinose, maltose, glycerol, lactose, fructose, galactose, and combinations thereof.

“Lyophilized composition”, “lyophilized pharmaceutical composition”, “lyophilized formulation”, or “solid composition” refers to a formulation containing a Clostridial toxin active ingredient which has been subjected to a lyophilization, freeze-drying or vacuum-drying process; and can be reconstituted with a reconstitution vehicle, such as for example saline or water, prior to administration to a patient. The lyophilized composition can be a freeze-dried composition or a vacuum-dried composition.

“Modified botulinum toxin” means a botulinum toxin that has had at least one of its amino acids deleted, modified, or replaced, as compared to a native botulinum toxin. Additionally, the modified botulinum toxin can be a recombinantly produced neurotoxin, or a derivative or fragment of a recombinantly made neurotoxin. A modified botulinum toxin retains at least one biological activity of the native botulinum toxin, such as, the ability to bind to a botulinum toxin receptor, or the ability to inhibit neurotransmitter release from a neuron. One example of a modified botulinum toxin is a botulinum toxin that has a light chain from one botulinum toxin serotype (such as serotype A), and a heavy chain from a different botulinum toxin serotype (such as serotype B). Another example of a modified botulinum toxin is a botulinum toxin coupled to a neurotransmitter, such as substance P.

“Molecular weight” as used herein is weight average molecular weight, which is measured and calculated as known in the art (e.g, Fred Billmeyer, Textbook of Polymer Science, 3^{rd} Edition, 1984, J. Wiley & Sons, pp. 16-19).

“Mutation” means a structural modification of a naturally occurring protein or nucleic acid sequence. For example, in the case of nucleic acid mutations, a mutation can be a deletion, addition or substitution of one or more nucleotides in the DNA sequence. In the case of a protein sequence mutation, the mutation can be a deletion, addition or substitution of one or more amino acids in a protein sequence. For example, a specific amino acid comprising a protein sequence can be substituted for another amino acid, for example, an amino acid selected from a group which includes the amino acids alanine, asparagine, cysteine, aspartic acid, glutamic acid, phenylalanine, glycine, histidine, isoleucine, lysine, leucine, methionine, proline, glutamine, arginine, serine, threonine, valine, tryptophan, tyrosine or any other natural or non-naturally occurring amino acid or chemically modified amino acids. Mutations to a protein sequence can be the result of mutations to DNA sequences that when transcribed, and the resulting mRNA translated, produce the mutated protein sequence. Mutations to a protein sequence can also be created by fusing a peptide sequence containing the desired mutation to a desired protein sequence.

“Non-cross-linked hyaluronic acid or salt thereof” refers to a non-cross-linked hyaluronic acid or salt thereof having a weight average molecular weight from 250 kDa to 2.4 MDa or from 4.6 MDa to 8 MDa.

“Patient” means a human or non-human subject receiving medical or veterinary care. Accordingly, the compositions as disclosed herein can be used in treating any animal, such as, for example, mammals, or the like.

“Peripherally administering” or “peripheral administration” means subdermal, intradermal, transdermal, or subcutaneous administration, but excludes intramuscular administration. “Peripheral” means in a subdermal location, and excludes visceral sites.

“Pharmaceutical composition” means a composition comprising an active pharmaceutical ingredient, such as, for example, a Clostridial toxin active ingredient such as a botulinum toxin, and at least one additional ingredient, such as, for example, a stabilizer or excipient or the like. A pharmaceutical composition is therefore a formulation which is suitable for diagnostic or therapeutic administration to a subject, such as a human patient. The pharmaceutical composition can be, for example, in a lyophilized or vacuum dried condition, a solution formed after reconstitution of the lyophilized or vacuum dried pharmaceutical composition, or as a solution or solid which does not require reconstitution.

“Pharmacologically acceptable excipient” is synonymous with “pharmacological excipient” or “excipient” and refers to any excipient that has substantially no long term or permanent detrimental effect when administered to mammal and encompasses compounds such as, e.g., stabilizing agent, a bulking agent, a cryo-protectant, a lyo-protectant, an additive, a vehicle, a carrier, a diluent, or an auxiliary. An excipient generally is mixed with an active ingredient, or permitted to dilute or enclose the active ingredient and can be a solid, semi-solid, or liquid agent. It is also envisioned that a pharmaceutical composition comprising a Clostridial toxin active ingredient can include one or more pharmaceutically acceptable excipients that facilitate processing of an active ingredient into pharmaceutically acceptable compositions. Insofar as any pharmacologically acceptable excipient is not incompatible with the Clostridial toxin active ingredient, its use in pharmaceutically acceptable compositions is contemplated. Non-limiting examples of pharmacologically acceptable excipients can be found in, e.g., Pharmaceutical Dosage Forms and Drug Delivery Systems (Howard C. Ansel et al., eds., Lippincott Williams & Wilkins Publishers, 7^{th} ed. 1999); Remington: The Science and Practice of Pharmacy (Alfonso R. Gennaro ed., Lippincott, Williams & Wilkins, 20^{th} ed. 2000); Goodman & Gilman's The Pharmacological Basis of Therapeutics (Joel G. Hardman et al., eds., McGraw-Hill Professional, 10^{th} ed. 2001); and Handbook of Pharmaceutical Excipients (Raymond C. Rowe et al., APhA Publications, 4^{th} edition 2003), each of which is hereby incorporated by reference in its entirety.

The constituent ingredients of a pharmaceutical composition can be included in a single composition (that is, all the constituent ingredients, except for any required reconstitution fluid, are present at the time of initial compounding of the pharmaceutical composition) or as a two-component system, for example a vacuum-dried composition reconstituted with a reconstitution vehicle which can, for example, contain an ingredient not present in the initial compounding of the pharmaceutical composition. A two-component system can provide several benefits, including that of allowing incorporation of ingredients which are not sufficiently compatible for long-term shelf storage with the first component of the two component system. For example, the reconstitution vehicle may include a preservative which provides sufficient protection against microbial growth for the use period, for example one-week of refrigerated storage, but is not present during the two-year freezer storage period during which time it might degrade the toxin. Other ingredients, which may not be compatible with a botulinum toxin or other ingredients for long periods of time, can be incorporated in this manner; that is, added in a second vehicle (e.g. in the reconstitution vehicle) at the approximate time of use. A pharmaceutical composition can also include preservative agents such as benzyl alcohol, benzoic acid, phenol, parabens and sorbic acid. Pharmaceutical compositions can include, for example, excipients, such as surface active agents; dispersing agents; inert diluents; granulating and disintegrating agents; binding agents; lubricating agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; antioxidants; stabilizing agents; and pharmaceutically acceptable polymeric or hydrophobic materials and other ingredients known in the art and described, for example in Genaro, ed., 1985, Remington's Pharmaceutical Sciences, Mack Publishing Co., Easton, Pa., which is incorporated herein by reference.

“Polysaccharide” means a polymer of more than two saccharide molecule monomers. The monomers can be identical or different.

“Stabilizing agent”, “stabilization agent” or “stabilizer” means a substance that acts to stabilize a Clostridial toxin active ingredient such that the potency of the pharmaceutical composition is increased relative to an unstabilized composition.

“Stabilizers” can include excipients, and can include protein and non-protein molecules.

“Surfactant” refers to a natural or synthetic amphiphilic compound. A surfactant can be non-ionic, zwitterionic, or ionic. Non-limiting examples of surfactants include a poloxamer, a polysorbate, and combinations thereof.

“Therapeutic formulation” means a formulation can be used to treat and thereby alleviate a disorder or a disease, such as, for example, a disorder or a disease characterized by hyperactivity (i.e. spasticity) of a peripheral muscle.

“Therapeutically effective concentration”, “therapeutically effective amount,” “effective amount,” “effective dose,” and “therapeutically effective dose” refer to the minimum dose of a Clostridial toxin active ingredient necessary to achieve the desired therapeutic effect and includes a dose sufficient to reduce a symptom associated with aliment being treated.

“TEM” as used herein, is synonymous with “Targeted Exocytosis Modulator” or “retargeted endopeptidase.” Generally, a TEM comprises an enzymatic domain from a Clostridial toxin light chain, a translocation domain from a Clostridial toxin heavy chain, and a targeting domain. The targeting domain of a TEM provides an altered cell targeting capability that targets the molecule to a receptor other than the native Clostridial toxin receptor utilized by a naturally-occurring Clostridial toxin. This re-targeted capability is achieved by replacing the naturally-occurring binding domain of a Clostridial toxin with a targeting domain having a binding activity for a non-Clostridial toxin receptor. Although binding to a non-Clostridial toxin receptor, a TEM undergoes all the other steps of the intoxication process including internalization of the TEM/receptor complex into the cytoplasm, formation of the pore in the vesicle membrane and di-chain molecule, translocation of the enzymatic domain into the cytoplasm, and exerting a proteolytic effect on a component of the SNARE complex of the target cell.

“Tonicity agent” means a low molecular weight excipient which is included in a formulation to provide isotonicity. Non-limiting examples of a tonicity agent include a disaccharide such as trehalose or sucrose; a polyalcohol such as sorbitol or mannitol; a monosaccharide such as glucose; and a salt, such as sodium chloride, calcium chloride, and potassium chloride.

“Topical administration” excludes systemic administration of the neurotoxin. In other words, and unlike conventional therapeutic transdermal methods, topical administration of botulinum toxin does not result in significant amounts, such as the majority of, the neurotoxin passing into the circulatory system of the patient.

“Treating” means to alleviate (or to eliminate) at least one symptom of a condition or disorder, such as, for example, wrinkles, spasticity, depression, pain (such as, for example, headache pain), bladder over activity, or the like, either temporarily or permanently.

As used herein, the term “unit” or “U” refers to the LD₅₀ dose or the dose determined by a cell based potency assay (CBPA). The LD₅₀ dose is defined as the amount of a Clostridial toxin active ingredient, Clostridial toxin complex or modified Clostridial toxin that killed 50% of the mice injected with the Clostridial toxin, Clostridial toxin complex or modified Clostridial toxin. The CBPA dose is determined as described in U.S. Pat. No. 8,618,261, the assay details of which are incorporated by reference herein. By way of example, about 50 picograms of a commercially available botulinum toxin type A (purified neurotoxin complex), which is available from Allergan, Inc., of Irvine, Calif. under the tradename BOTOX® in 100 unit vials, has a LD₅₀ in mice (i.e. 1 unit). One unit of BOTOX® contains about 50 picograms (about 56 attomoles) of botulinum toxin type A complex, where one unit (U) corresponds to the LD₅₀ upon intraperitoneal injection into female Swiss Webster mice weighing 18 to 20 grams each.

“Variant” means a Clostridial neurotoxin, such as wild-type botulinum toxin serotype A, B, C, D, E, F or G, that has been modified by the replacement, modification, addition or deletion of at least one amino acid relative to wild-type botulinum toxin, which is recognized by a target cell, internalized by the target cell, and catalytically cleaves a SNARE (SNAP (Soluble NSF Attachment Protein) Receptor) protein in the target cell.

An example of a variant neurotoxin component can comprise a variant light chain of a botulinum toxin having one or more amino acids substituted, modified, deleted and/or added. This variant light chain may have the same or better ability to prevent exocytosis, for example, the release of neurotransmitter vesicles. Additionally, the biological effect of a variant may be decreased compared to the parent chemical entity. For example, a variant light chain of a botulinum toxin type A having an amino acid sequence removed may have a shorter biological persistence than that of the parent (or native) botulinum toxin type A light chain.

### Pharmaceutical Compositions

The pharmaceutical composition disclosed herein comprise a Clostridial toxin active ingredient and a non-cross-linked hyaluronic acid, or salt thereof, with a weight average molecular weight of less than about 2.5 MDa or less than about 2.4 MDa. In a preferred embodiment, the non-cross-linked hyaluronic acid, or salt thereof, has a weight average molecular weight of between about 250 kDa and about 2.5 MDa. In another embodiment, the non-cross-linked hyaluronic acid, or salt thereof, has a weight average molecular weight of between about 4.5 MDa and about 8 MDa. In still other embodiments, the non-cross-linked hyaluronic acid, or salt thereof, has a weight average molecular weight of between about 500 kDa-5000 kDa, about 500 kDa to less than about 2500 kDa or about 500 kDa to about 2000 kDa. In one embodiment, the pharmaceutical composition is free of cross-linked hyaluronic acid, including salts thereof. That is, the pharmaceutical composition does not comprise a cross-linked hyaluronic acid or salt thereof.

The composition can be prepared by mixing a composition comprising Clostridial toxin active ingredient with a composition comprising a non-cross-linked hyaluronic acid or salt thereof and a diluent that is not a non-cross-linked hyaluronic acid or salt thereof, or by mixing a non-cross-linked hyaluronic acid or salt thereof with a composition comprising Clostridial toxin active ingredient and a diluent that is not a non-cross-linked hyaluronic acid or salt thereof, or by mixing Clostridial toxin active ingredient, a non-cross-linked hyaluronic acid or salt thereof, and the diluent together.

In a first aspect, a pharmaceutical composition comprising (or consisting of, or consisting essentially of) a Clostridial toxin active ingredient, a non-cross-linked hyaluronic acid or salt thereof, a disaccharide, a surfactant and an antioxidant is described. The composition can, in one embodiment, be a solid composition, such as a lyophilized powder that is reconstituted prior to use. In another embodiment, the composition is a liquid composition; that is, the composition is manufactured and stored in liquid form. Studies were conducted demonstrating that the compositions show extended duration of effect of the Clostridial toxin active ingredient compared with compositions lacking a non-cross-linked hyaluronic acid or salt thereof.

In a first study, described in Example 1, liquid compositions were prepared that comprised botulinum toxin as a model Clostridial toxin active ingredient, a non-cross-linked hyaluronic acid or salt thereof, a disaccharide, a surfactant, and an antioxidant. The compositions were prepared by mixing sodium hyaluronate powder having different molecular weights and intrinsic viscosities with disaccharide, a surfactant and an antioxidant to from a hyaluronic acid-containing composition, and then mixing into the hyaluronic acid-containing composition a botulinum toxin solution to prepare the botulinum toxin-hyaluronic acid formulations with various concentrations of botulinum toxin. Table 1 summarizes the molecular weights or intrinsic viscosities of the hyaluronic acid in each composition along with the other ingredients in the composition.

Table 2 summarizes the compositions with botulinum toxin and the hyaluronic acid composition, and additionally summarizes the ingredients in several comparative, control compositions comprising botulinum toxin with no non-cross-linked hyaluronic acid or salt thereof (without any hyaluronic acid or salt thereof).

TABLE 1
Compositions of hyaluronic acid/sodium hyaluronate (HA)

| | | | | | ²Viscosity |
|---|---|---|---|---|---|
| | HA MW or intrinsic | | HA | | @ 0.1/s |
| Formulation | viscosity from CofA | | wt % | Diluent | (Pa-s) |
| 1.2% HA-1500 kDa | ~1580 | kDa | 1.2% | 20 mM histidine, pH 6, 8% trehalose, | ~34.1 to 50.4³ |
| | | | | 4% poloxamer 188, and 0.2% methionine | ~92-110⁴ |
| 2% HA 1500 kDa | ~1580 | kDa | 2% | 20 mM histidine, pH 6, 8% trehalose, | ~511 to 567⁴ |
| | | | | 4% poloxamer 188, and 0.2% methionine | |
| 3% HA-LMW¹ | ~0.85 | m³/kg | 3% | 20 mM histidine, pH 6, 8% trehalose, | 11.7 |
| | | | | 4% poloxamer 188, and 0.2% methionine | |
| 5% HA-LMW¹ | ~0.85 | m³/kg | 5% | 20 mM histidine, pH 6, 8% trehalose, | 74.0 |
| | | | | 4% poloxamer 188, and 0.2% methionine | |
| 0.6% HA-HHMW¹ | ~3.44 | m³/kg | 0.6% | 20 mM histidine, pH 6, 8% trehalose, | 48.9 |
| | | | | 4% poloxamer 188, and 0.2% methionine | |
| 1.35% HA-HMW | ~3.03 | m³/kg | 1.35% | 20 mM histidine, pH 6, 8% trehalose, | 142 |
| | | | | 4% poloxamer 188, and 0.2% methionine | |
| 0.6% HA-HMW | ~3.03 | m³/kg | 0.6% | 20 mM histidine, pH 6, 8% trehalose, | 19.7 |
| | | | | 4% poloxamer 188, and 0.2% methionine | |
| 0.4% HA-HMW | ~2.87 | m³/kg | 0.4% | 20 mM histidine, pH 6, 8% trehalose, | 14.2 |
| | | | | 4% poloxamer 188, and 0.2% methionine | |

¹Abbreviations: LMW: low molecular weight, HMW: high molecular weight, HHMW: very high molecular weight.
²Viscosity determined at 25° C and at a shear rate of about 0.1/second (Example 1).
³Weight average MW measured as described herein was 1316 kDa.
⁴Weight average MW measured as described herein was 1568 kDa.

TABLE 2
Botulinum toxin-HA Compositions and Control
(comparison) Compositions with no HA

| Botulinum toxin dose | | | |
|---|---|---|---|
| (per kg of the rat) | BoNT/A | HA | |
| w or w/o HA | (U/mL) | (mg/mL) | Diluent |
| 2.96 U/kg control | 17.9 | 0 | 20 mM histidine, |
| (“Ctl”) | | | pH 6, 8% trehalose, |
| 4.70 U/kg control | 28.2 | 0 | 4% poloxamer 188, |
| (“Ctl”) | | | and 0.2% methionine |
| 9.40 U/kg control | 56.4 | 0 | |
| (“Ctl”) | | | |
| 11.75 U/kg control | 70.5 | 0 | |
| (“Ctl”) | | | |
| 16.45 U/kg control | 98.7 | 0 | |
| (“Ctl”) | | | |
| 2.96 U/kg in 1.2% | 17.9 | 12 | HA-1500 kDa in |
| HA-1500 kDa | | | 20 mM histidine, |
| 4.70 U/kg in 1.2% | 28.2 | 12 | pH 6, 8% trehalose, |
| HA-1500 kDa | | | 4% poloxamer 188, |
| 9.40 U/kg in 1.2% | 56.4 | 12 | and 0.2% methionine |
| HA-1500 kDa | | | |
| 11.75 U/kg in 1.2% | 70.5 | 12 | |
| HA-1500 kDa | | | |
| 4.70 U/kg in 2% | 28.2 | 20 | |
| HA-1500 kDa | | | |
| 4.70 U/kg in 3% | 28.2 | 30 | HA-LMW in 20 mM |
| HA-LMW | | | histidine, pH 6, |
| | | | 8% trehalose, 4% |
| | | | poloxamer 188, and |
| | | | 0.2% methionine |
| 4.70 U/kg in 0.6% | 28.2 | 6 | HA-HHMW in 20 mM |
| HA-HHMW | | | histidine, pH 6, |
| | | | 8% trehalose, 4% |
| | | | poloxamer 188, and |
| | | | 0.2% methionine |

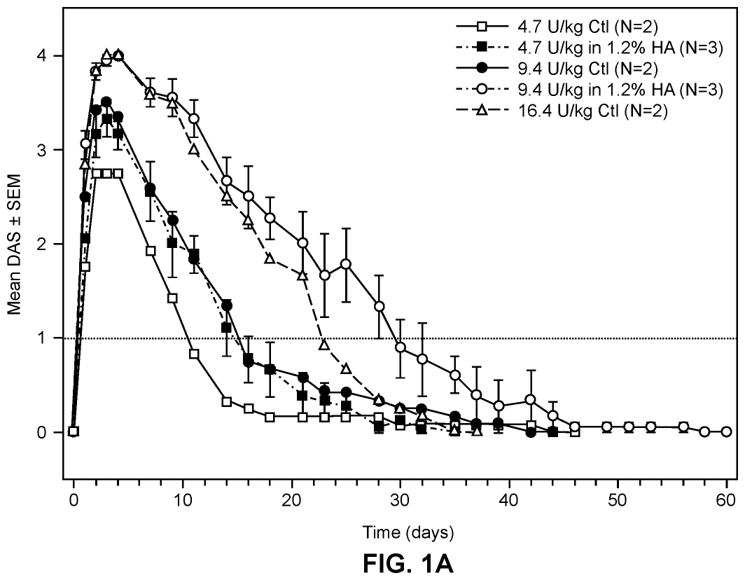

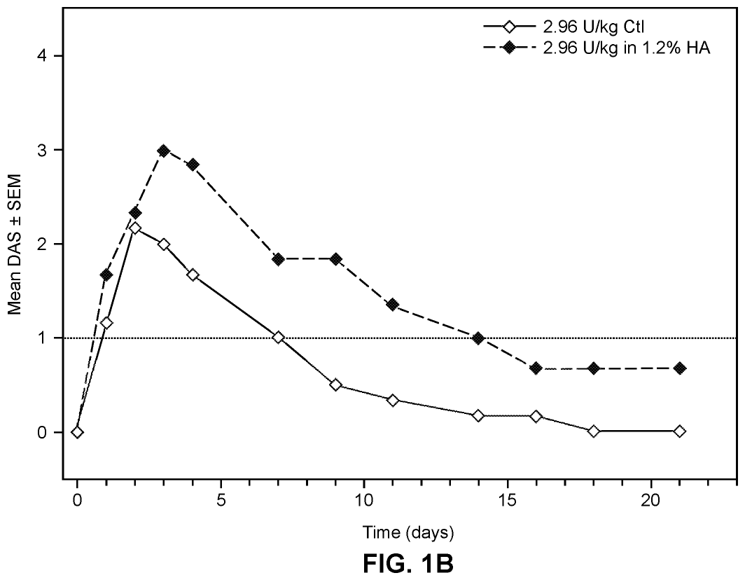

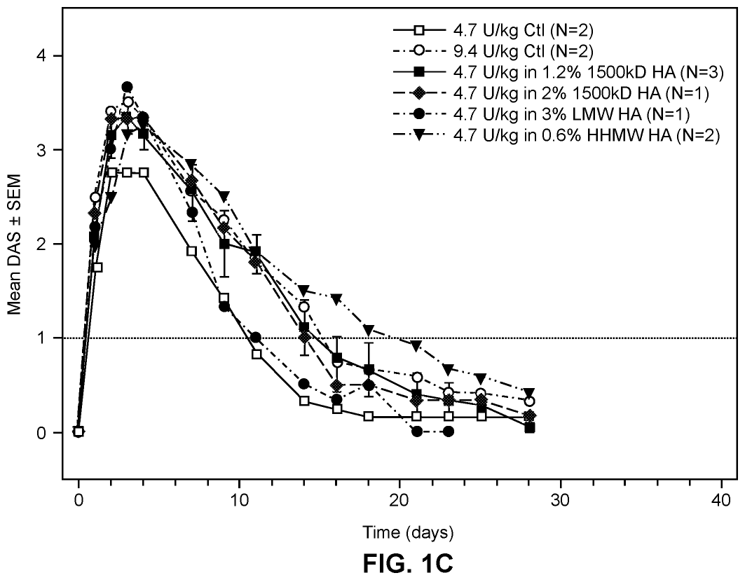

The botulinum toxin-hyaluronic acid formulations of Table 2 were tested in vivo for duration of effect. As described in Example 2, test and control formulations were injected intramuscular into the tibialis anterior of a rat. The rat DAS (digital abduction score) assay was used to assess efficacy of the formulation, where rat paralysis is assessed by a DAS response which is scored from 0 to 4, with 4 representing maximum paralysis. The results are shown in FIGS. 1A-1C.

FIG. 1A shows the average DAS as a function of time, in days, following injection of the test and control (comparator) compositions. The control compositions, lacking non-cross-linked hyaluronic acid, comprised botulinum toxin type A (BoNT/A) at concentrations of 28.2 U/mL, 56.4 U/mL, and 98.7 U/mL in the diluent noted in Table 2 above (20 mM histidine, pH 6, 8% trehalose, 4% poloxamer 188, and 0.2% methionine). These control compositions were injected in the rat to give a BoNT/A dose, respectively of 4.7 U/kg (open squares), 9.4 U/kg (open circles), and 16.4 U/kg (open triangles). The compositions with linear, non-cross-linked hyaluronic acid were composed of 1.2% HA (weight average MW of 1500 kDa) and had BoNT/A at 28.2 U/mL and 56.4 U/mL in the same diluent as the control compositions (20 mM histidine, pH 6, 8% trehalose, 4% poloxamer 188, and 0.2% methionine). These compositions were injected in the rat to give a BoNT/A dose, respectively, of 4.7 U/kg (closed squares) and 9.4 U/kg (closed circles). The data in FIG. 1A shows that compositions with a linear, non-cross-linked hyaluronic acid improved the duration of effect of the botulinum toxin, as evidenced by the increased rat DAS peak response and duration in the animals treated with compositions comprising a linear, non-cross-linked hyaluronic acid. An approximately two-fold apparent dose increase was provided when linear, non-cross-linked hyaluronic acid was included in the compositions.

FIG. 1B shows the results for compositions with a lower concentration of BoNT/A. The test and control compositions both had a concentration of 17.9 U/mL and were dosed via injection to 2.96 U/kg. The test composition (closed diamonds) comprised 1.2 wt % of a linear, non-cross-linked hyaluronic acid (weight average MW of 1500 kDa), whereas the comparative control composition (open diamonds) had no linear, non-cross-linked hyaluronic acid. An increased response and an extended duration of effect is observed in the DAS scores for the animals treated with the composition comprising linear, non-cross-linked hyaluronic acid compared to the animals treated with the comparator, control compositions. An approximately two-fold increase in duration (defined as time to return to rat DAS score of 1) was observed for the animals treated with composition comprising linear, non-cross-linked hyaluronic acid compared to the animals treated with the comparator, control compositions.

FIG. 1C shows data from in vivo testing of compositions with different amounts and different molecular weights of linear, non-cross-linked hyaluronic acid. Compositions with BoNT/A were dosed at 4.7 U/kg, except for one comparator formulation dosed at 9.4 U/kg (open circles). The test compositions (Table 2 above) included compositions with 1.2% non-cross-linked hyaluronic acid (1500 kDa; closed squares), 2% non-cross-linked hyaluronic acid (1500 kDa, closed diamonds), 3% non-cross-linked hyaluronic acid (LMW, closed circles), and 0.6% non-cross-linked hyaluronic acid (HHMW, closed inverted triangles). The comparator, control compositions lacked non-cross-linked hyaluronic acid and were dosed at 4.7 U/kg BoNT/A (open squares) or at 9.4 U/kg (open circles). The compositions comprising linear, non-cross-linked hyaluronic acid provided an increased response and an extended duration of effect as evident from the higher DAS scores relative to the control compositions, and the higher DAS scores for a longer period of time. The extended duration of effect is evident from the longer period of time required for the DAS score to return a score of 1 for the animals treated with the compositions comprising linear, non-cross-linked hyaluronic acid.

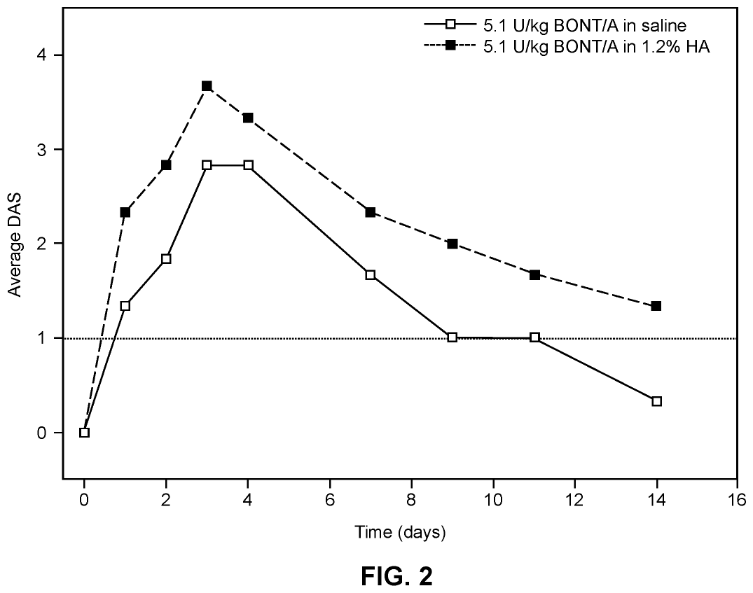

In another study, described in Example 3, compositions with botulinum toxin were prepared by reconstituting powdered botulinum toxin (BOTOX®) with isotonic saline (0.9 wt % sodium chloride) as a control. A test composition was similarly prepared that additionally comprised 1.2 wt % non-cross-linked hyaluronic acid (MW 1500 kDa) in 20 mM histidine, pH 6, 8% trehalose, 4% poloxamer 188, and 0.2% methionine. The effect and duration of effect of the compositions was tested using the DAS assay and the results are shown in FIG. 2. The test and control compositions, both dosed at 5.1 U/kg, reached maximum paralysis about 3 days after injection. The composition with non-cross-linked hyaluronic acid (closed squares) was more effective at the same dose, as evidenced by the 40% increase in average DAS at the peak paralysis. The composition with non-cross-linked hyaluronic acid (closed squares) also achieved a longer duration of effect as evidenced by the control composition (open squares) returning to baseline 1 DAS at day 9 and the test composition (closed squares) returning to baseline 1 DAS sometime after about day 16. This translates into a nearly two fold improvement in duration of effect and an at least about 25% increase in effect provided from the same dose of botulinum toxin when provided in a composition comprising a non-cross-linked hyaluronic acid or salt thereof.

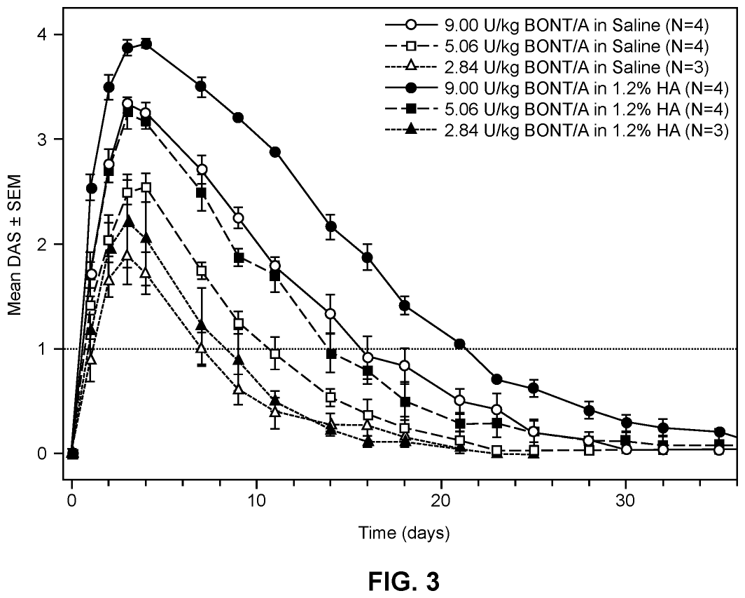

Additional compositions were prepared and evaluated for duration of effect and extent of effect using the DAS assay. These compositions and studies are now described with reference to FIG. 3-FIG. 8.

A study was designed to compare the effect of adding a non-cross-linked hyaluronic acid to the commercially available BoNT/A sold under the tradename BOTOX®. Powdered botulinum toxin (BOTOX®) was reconstituted with isotonic saline (0.9 wt % sodium chloride), for dosing at various BoNT/A dose levels as a control. Powdered botulinum toxin (BOTOX®) was reconstituted with saline and then combined with 20 mM histidine, pH 6, 8% trehalose, 4% poloxamer 188, and 0.2% methionine with 1.2 wt % non-cross-linked hyaluronic acid (1500 kDa MW), as the test composition. The control and tests compositions were injected intramuscularly into the tibialis anterior of the rat at BoNT/A doses of 2.84 U/kg, 5.06 U/kg and 9.00 U/kg. Rat paralysis was assessed by the DAS response using a score from 0 to 4, with 4 representing maximum paralysis. FIG. 3 shows the mean DAS as a function of time, in days, following injection if the compositions, where each N is an independent study comprised of 6 rats/dose. Botulinum toxin compositions lacking a non-cross-linked hyaluronic acid administered with BoNT/A doses of 9 U/kg (open circles), 5.06 U/kg (open squares), or 2.84 U/kg (open triangles) were less effective in terms of paralysis and had a shorter duration of effect compared with compositions comprising a non-cross-linked hyaluronic acid or salt thereof (1500 kDa) administered at the same BoNT/A doses of 9.0 U/kg (closed circles), 5.06 U/kg (closed squares), or 2.84 U/kg (closed triangles). At a BoNT/A dose of 9.0 U/kg, the composition comprising non-cross-linked hyaluronic acid provided 21 days of effect (duration of effect defined as number of days for DAS score to return to a score of 1), whereas the control composition without non-cross-linked hyaluronic acid administered a 9.0 U/kg provided 15.5 days of effect. Thus, in one embodiment, compositions with a botulinum toxin and a non-cross-linked hyaluronic acid improve duration of effect by at least about 30%, 35%, or 40% relative to a botulinum toxin composition with no non-cross-linked hyaluronic acid when administered via the same route of administration at the same toxin dose or potency.

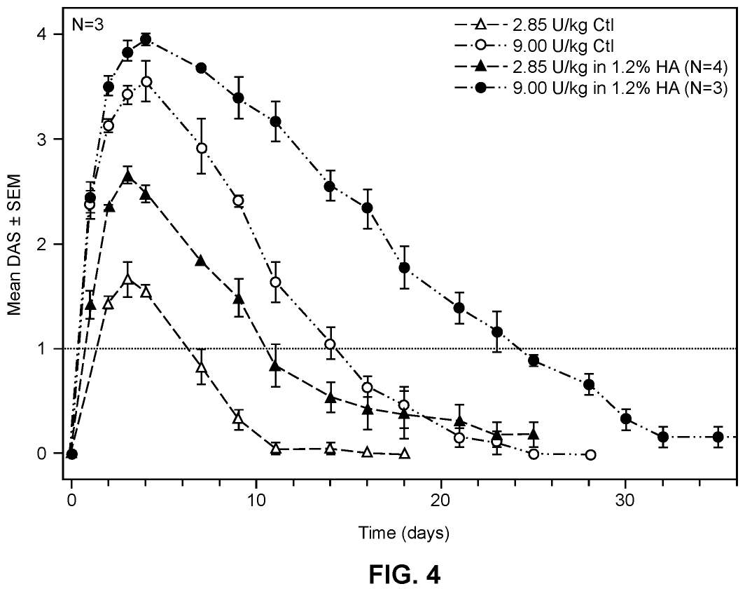

Compositions with BoNT/A in 20 mm histidine, pH 6, 8 wt % trehalose, 4 wt % surfactant (Poloxamer P-188), and 0.2 wt % methionine and with 1.2 wt % non-cross-linked hyaluronic acid (1500 kDa MW) or without non-cross-linked hyaluronic acid were prepared. The compositions were injected intramuscularly into the tibialis anterior of the rat at BoNT/A doses of 2.85 U/kg and 9 U/kg. Rat paralysis was assessed by the DAS response using a score from 0 to 4, with 4 representing maximum paralysis. FIG. 4 shows the mean DAS as a function of time, in days, following injection if the compositions, where each N is an independent study comprised of 6 rats/dose. Botulinum toxin compositions comprising a non-cross-linked hyaluronic acid provided an improved effect and a longer duration of effect. The composition with non-cross-linked hyaluronic acid and dosed at 2.85 U/kg (closed triangles) had a peak DAS of about 2.5, compared to the composition with no non-cross-linked hyaluronic acid at the same dose (open triangles) with a peak DAS of about 1.6. At a BoNT/A dose of 9 U/kg, the composition comprising non-cross-linked hyaluronic acid (closed circles) provided 24 days of effect (duration of effect defined as number of days for DAS score to return to a score of 1), whereas the control composition without non-cross-linked hyaluronic acid administered a 9 U/kg (open circles) provided 14.5 days of effect. Thus, in one embodiment, compositions with a botulinum toxin and a non-cross-linked hyaluronic acid improve duration of effect by at least about 30%, 35%, 40% or 45% relative to a botulinum toxin composition with no non-cross-linked hyaluronic acid when administered via the same route of administration at the same toxin dose or potency.

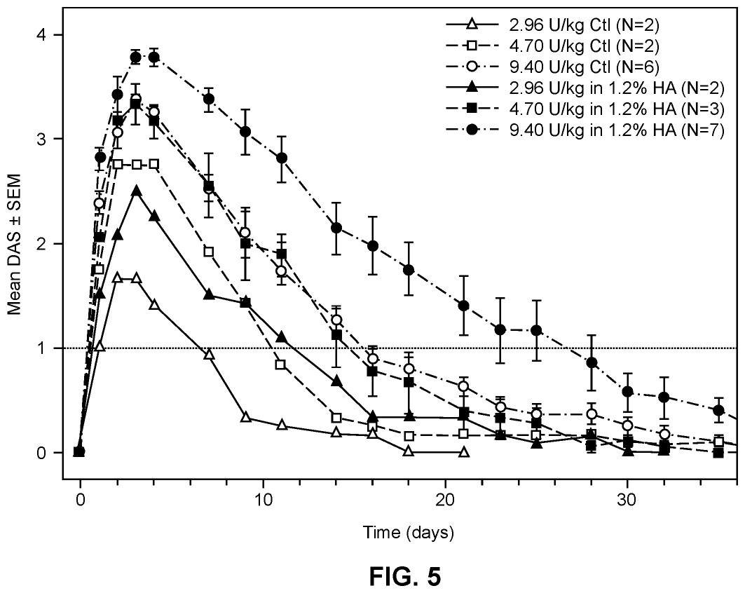

Another study evaluated effectiveness and duration of effect at toxin dose levels ranging from 2.96 U/kg to 9.4 U/kg. Compositions of BoNT/A, 20 mm histidine, pH 6, 8 wt % trehalose, 4 wt % surfactant (Poloxamer P-188), and 0.2 wt % methionine and with 1.2 wt % non-cross-linked hyaluronic acid (1500 kDa MW) or without non-cross-linked hyaluronic acid were prepared. The compositions were injected intramuscularly into the tibialis anterior of the rat at BoNT/A doses of 2.96 U/kg, 4.70 U/kg and 9.40 U/kg. Rat paralysis was assessed by the DAS response using a score from 0 to 4, with 4 representing maximum paralysis. FIG. 5 is a graph of mean DAS as a function of time, in days, following injection into rats of botulinum toxin control compositions lacking a non-cross-linked hyaluronic acid and administered with BoNT/A doses of 2.96 U/kg (open triangles), 4.70 U/kg (open squares), or 9.4 U/kg (open circles) or with compositions comprising 1.2 wt % non-cross-linked hyaluronic acid (1500 kDa) administered at BoNT/A doses of 2.96 U/kg (closed triangles), 4.70 U/kg (closed squares), or 9.4 U/kg (closed circles). The compositions with non-cross-linked hyaluronic acid provided a longer duration of effect in addition to a greater effect. Thus, compositions with a botulinum toxin and a non-cross-linked hyaluronic acid that provide a 1.5 fold, 1.7 fold or 2 fold longer duration of effect are provided. In other embodiments, compositions with a botulinum toxin and a non-cross-linked hyaluronic acid that provide a 1.2, 1.3, or 1.4 fold longer duration of effect are provided.

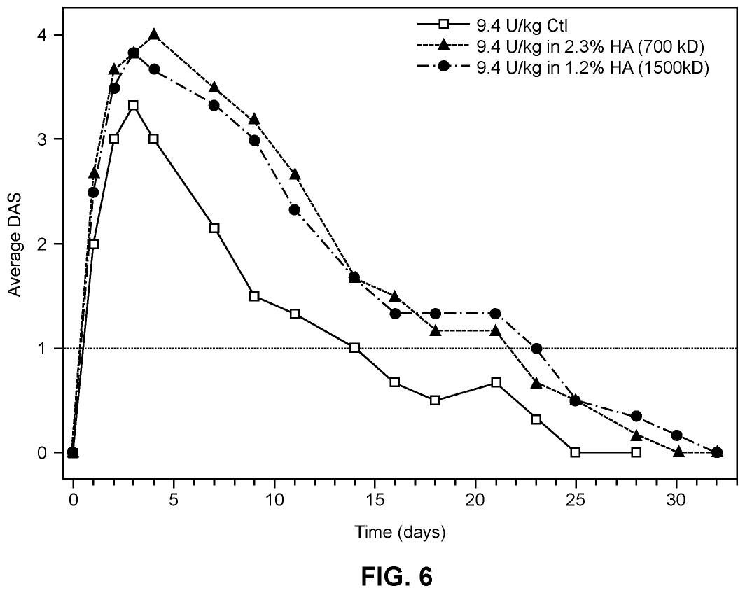

The impact on duration of effect and extent of effect due to molecular weight or intrinsic viscosity of the non-cross-linked hyaluronic acid was evaluated by preparing BoNT/A compositions with 1.2 wt % non-cross-linked hyaluronic acid 1500 kDa average MW or 2.3 wt % 700 kDa average MW. The compositions in this study additionally comprised 20 mM histidine, pH 6, 8 wt % trehalose, 4 wt % surfactant (Poloxamer P188) and 0.2 wt % methionine. The compositions were injected intramuscularly into the tibialis anterior of the rat at BoNT/A dose of 9.4 U/kg. Rat paralysis was assessed by the DAS response using a score from 0 to 4, with 4 representing maximum paralysis. FIG. 6 shows the average DAS as a function of time, in days, following injection into rats. The composition with no non-cross-linked hyaluronic acid (control, open squares) provided a 15 day period of effect (defined as return to DAS=1), whereas the compositions with 1.2 wt % non-cross-linked hyaluronic acid with 1500 kDa average MW (closed circles) or 2.3 wt % 700 kDa average MW (closed triangles) achieved a 22 and 24 day duration of effect, respectively. Consistently, compositions with non-cross-linked hyaluronic acid extend the duration of effect of botulinum toxin by about 1.5 fold to 2 fold relative to compositions with no non-cross-linked hyaluronic acid.

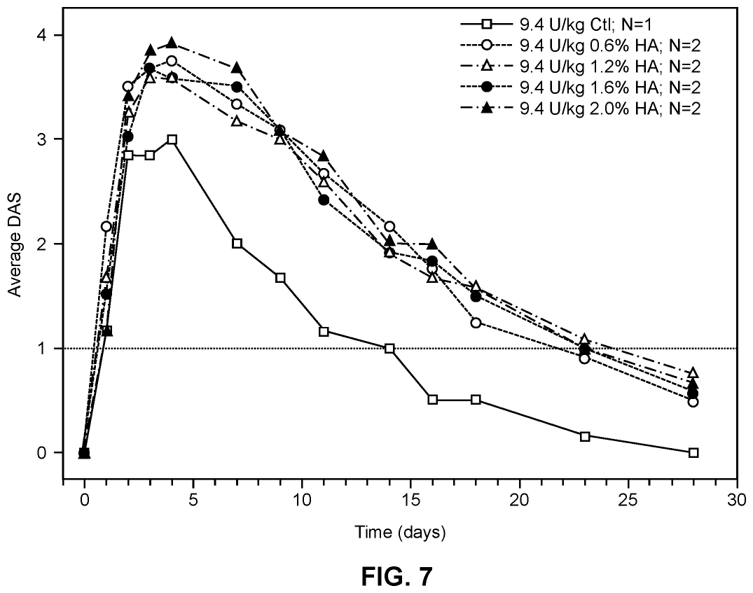

Compositions with different concentrations of non-cross-linked hyaluronic acid (with 1500 kDa average MW) were prepared. In this study, the compositions comprised BoNT/A, 20 mM histidine, pH 6, 8 wt % trehalose, 4 wt % surfactant (Poloxamer P188), 0.2 wt % methionine and no non-cross-linked hyaluronic acid (control, open squares) or 0.6 wt % (open circles), 1.2 wt % (open triangles), 1.6 wt % (closed circles), or 2.0 wt % (closed triangles) non-cross-linked hyaluronic acid. The compositions were injected intramuscularly into the tibialis anterior of the rat at BoNT/A dose of 9.4 U/kg. Rat paralysis was assessed by the DAS response using a score from 0 to 4, with 4 representing maximum paralysis. FIG. 7 is a graph of average DAS as a function of time, in days, following injection into rats of these botulinum toxin compositions, dosed at 9.4 U/kg. It is observed that the longer duration of effect provided by non-cross-linked hyaluronic acid is not dependent on the percent of non-cross-linked hyaluronic acid, as the compositions with 0.6-2.0 wt % non-cross-linked hyaluronic acid all provided a longer duration of effect compared to the control composition with no non-cross-linked hyaluronic acid. Viscosity of the compositions was measured at a shear rate of 0.1/sec at 25° C., and found to range from 10 Pa-s to 580 Pa-s. Accordingly, in one embodiment, compositions with a non-cross-linked hyaluronic acid or salt thereof and a botulinum toxin that have a viscosity of between about 1-1000 Pa-s or between about 1-750 Pa-s provide a duration of effect that is 50%, 60%, 75%, 80% or 100% greater than a similar composition with no non-cross-linked hyaluronic acid administered at the same toxin dose and same administration route.

In another study, compositions with BoNT/A and with 1.2 wt % non-cross-linked hyaluronic acid (1500 kDa MW) were prepared from various diluents. Table A below summarizes the compositions.

TABLE A
BoNT/A Compositions for Study in FIG. 12

| | Hyaluronic | | | | | |
|---|---|---|---|---|---|---|
| Formulation | Acid (1500 | | | | | |
| No. | kDa) | Buffer | pH | Sugar | Surfactant | Antioxidant |
| 1 (control) | None | 20 mM | pH = 6 | 8 wt % | 4 wt % | 0.2 wt % |
| | | histidine | | trehalose | Poloxamer | methionine |
| | | | | | P188 | |
| 2 | 1.2 wt % | 20 mM | pH = 6 | 8 wt % | 4 wt % | 0.2 wt % |
| | | histidine | | trehalose | Poloxamer | methionine |
| 3 | 1.2 wt % | 50 mM | pH = 6 | 8 wt % | 4 wt % | 0.2 wt % |
| | | histidine | | trehalose | Poloxamer | methionine |
| | | | | | P188 | |
| 4 | 1.2 wt % | 10 mM | pH = 6 | 8 wt % | 4 wt % | 0.2 wt % |
| | | histidine | | trehalose | Poloxamer | methionine |
| | | | | | P188 | |
| 5 | 1.2 wt % | 20 mM | pH = 5 | 8 wt % | 4 wt % | 0.2 wt % |
| | | histidine | | trehalose | Poloxamer | methionine |
| | | | | | P188 | |
| 6 | 1.2 wt % | 20 mM | pH = 6 | none | 4 wt % | 0.2 wt % |
| | | histidine | | | Poloxamer | methionine |
| | | | | | P188 | |
| 7 | 1.2 wt % | 20 mM | pH = 6 | none | 0.6 wt % | 0.2 wt % |
| | | histidine | | | Poloxamer | methionine |
| | | | | | P188 | |

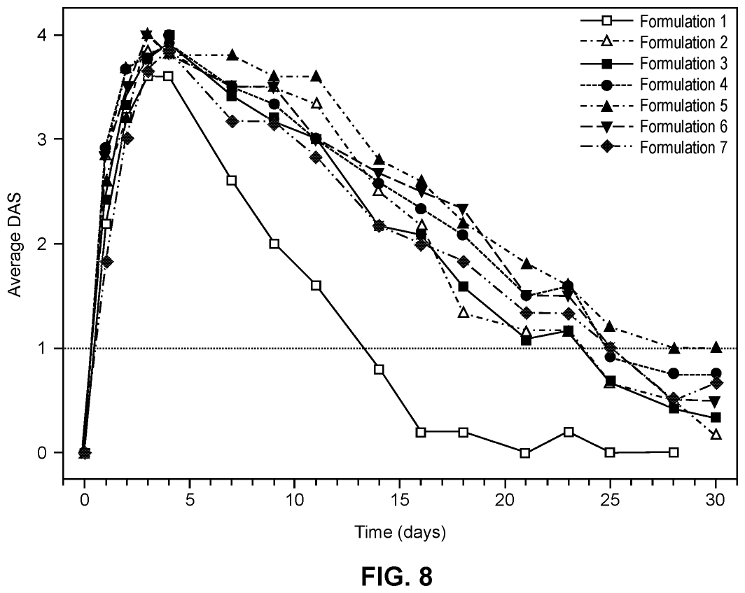

The compositions in Table A were injected intramuscularly into the tibialis anterior of the rat at BoNT/A dose of 9.4 U/kg. Rat paralysis was assessed by the DAS response using a score from 0 to 4, with 4 representing maximum paralysis. FIG. 8 is a graph of average DAS as a function of time, in days, following injection into rats of the botulinum toxin compositions, where the formulation number assigned in Table A corresponds to the symbols as follows: Formulation 1 (control, no non-cross-linked hyaluronic acid), open squares; Formulation 2, open triangles; Formulation 3, closed squares; Formulation 4, closed circles; Formulation 5, closed triangles; Formulation 6, inverted triangles; Formulation 7, closed diamonds. It was observed that the longer duration of effect provided by non-cross-linked hyaluronic acid is achieved for all the various vehicles. Formulation 5 (closed triangles) was adjusted to pH 5 (all other formulations were pH 6) and provided the longest duration of effect. Accordingly, in one embodiment, compositions with a non-cross-linked hyaluronic acid or salt thereof and a botulinum toxin that have a pH of between about 4.70-6.50, 4.75-6.50, 4.80-6.50, 4.85-6.50, 4.90-6.50, 4.95-6.50, 4.98-6.50, 4.99-6.50, 5.00-6.50, 4.70-6.30, 4.75-6.30, 4.80-6.30, 4.85-6.30, 4.90-6.30, 4.95-6.30, 4.98-6.30, 4.99-6.30, 5.00-6.30, 4.70-6.20, 4.75-6.20, 4.80-6.20, 4.85-6.20, 4.90-6.20, 4.95-6.20, 4.98-6.20, 4.99-6.20, 5.00-6.20, 4.70-6.10, 4.75-6.10, 4.80-6.10, 4.85-6.10, 4.90-6.10, 4.95-6.10, 4.98-6.10, 4.99-6.10, 5.00-6.10, or 5.00-6.00 are contemplated. These compositions at a selected pH provide a duration of effect that is 50%, 60%, 75%, 80% or 100% greater than a similar composition with no non-cross-linked hyaluronic acid administered at the same toxin dose and same administration route.

Accordingly, a pharmaceutical composition comprising a Clostridial toxin active ingredient, such as botulinum toxin, about 1.2 wt % of a non-cross-linked hyaluronic acid or salt thereof with weight average molecular weight of from about 450 kDa to 2.0 MDa, such as from 450 kDa to 1.6 MDa MDa (further such as about 1.4-1.6 MDa), trehalose, poloxamer, and methionine is provided. The pharmaceutical composition can be albumin free and does not comprise cross-linked hyaluronic acid or salt thereof. Such pharmaceutical composition can increase the efficacy and/or duration time of Clostridial toxin active ingredient by at least 10%, 20%, 30%, 40%, 50%, 60%, 70%, 80%, 90%, and 100% as compared to a pharmaceutical composition which does not comprise a non-cross-linked hyaluronic acid or salt thereof.

In one embodiment, the pharmaceutical composition as disclosed herein increases the efficacy and/or duration time of Clostridial toxin active ingredient by at least 10%, 20%, 30%, 40%, 50%, 60%, 70%, 80%, 90%, and 100% as compared to a pharmaceutical composition which does not comprise a non-cross-linked hyaluronic acid or salt thereof.

In another embodiment, the pharmaceutical composition as disclosed herein can be prepared by mixing a composition comprising a non-cross-linked hyaluronic acid or salt thereof with a lyophilized formulation comprising botulinum toxin, a disaccharide, a surfactant, and an antioxidant, or by mixing a composition comprising botulinum toxin with a lyophilized formulation comprising a non-cross-linked hyaluronic acid or salt thereof, a disaccharide, a surfactant, and an antioxidant. In one embodiment, the lyophilized composition can comprise botulinum toxin and/or a non-cross-linked hyaluronic acid or salt thereof, trehalose, a polysorbate or poloxamer, and methionine or NAC. In another embodiment, the lyophilized composition can comprise botulinum toxin and/or a non-cross-linked hyaluronic acid or salt thereof, a disaccharide; a surfactant selected from a poloxamer and a polysorbate, and an antioxidant selected from methionine, NAC, EDTA, EGTA, DTPA, analogs thereof, and combinations thereof. In another embodiment, the lyophilized composition can comprise botulinum toxin and/or a non-cross-linked hyaluronic acid or salt thereof, trehalose, a poloxamer surfactant (e.g. KOLLIPHOR® P-188) and methionine as a stabilizing antioxidant. Table 3 lists components in exemplary lyophilized formulations.

TABLE 3
Components in exemplary lyophilized compositions

| | Excipient, % w/w | | | | | | |
|---|---|---|---|---|---|---|---|
| | | TWEEN ® | P | | | | |
| Formulation | Treh | 20 | 188 | NaCl | Met | NAC | Buffer |
| Formulation 1 | 2 | — | 4 | — | — | 0.03 | 20 mM Histidine |
| | | | | | | | pH 5.5 |
| Formulation 2 | 2 | | 4 | | 0.2 | | 20 mM Histidine |
| | | | | | | | pH 6.0 |
| Formulation 3 | 8 | 0.04 | | | | 0.03 | 20 mM Histidine |
| | | | | | | | pH 6.0 |
| Formulation 4 | 8 | | 0.6 | | 0.2 | | 20 mM Histidine |
| | | | | | | | pH 6.0 |

Each formulation additionally comprises a clostridial toxin active ingredient and/or a non-cross-linked hyaluronic acid or salt thereof; Treh=trehalose; P 188=poloxamer P 188; Met=L-methionine; NAC=N-acetyl-L-cysteine.

In another embodiment, liquid compositions comprised of a Clostridial toxin active ingredient, a non-cross-linked hyaluronic acid or salt thereof, a tonicity agent, a surfactant, and an antioxidant can be prepared. The liquid solutions can be prepared by mixing a non-cross-linked hyaluronic acid or salt thereof with a liquid solution comprising a Clostridial toxin active ingredient, using botulinum toxin as a model, a disaccharide tonicity agent, a poloxamer surfactant, and an antioxidant, or by mixing a composition comprising a Clostridial toxin active ingredient, using botulinum toxin as a model, with a liquid composition comprising a non-cross-linked hyaluronic acid or salt thereof, a disaccharide tonicity agent, a poloxamer surfactant, and an antioxidant. The disaccharide tonicity agent can be trehalose and the poloxamer surfactant can be poloxamer P188. Three exemplary formulations are shown in Table 4, each with the same amount of botulinum toxin (100 units/mL) and a non-cross-linked hyaluronic acid or salt thereof (0.2 to 10 w/w %), 8 w/w % trehalose, and 4 w/w % poloxamer P188 in histidine buffer. Formulation 10 had no antioxidant; Formulation 11 contained NAC, and Formulation 12 contained L-methionine.

TABLE 4

| | Diluent, % w/w | | | |
|---|---|---|---|---|
| | | treha- | poloxamer | |
| Formulation No.* | Antioxidant | lose | P188 | buffer |
| Formulation 10 | None | 8 | 4 | histidine |
| Formulation 11 | N-acetyl-L- | 8 | 4 | histidine |
| | cysteine, | | | |
| | 0.2% w/w | | | |
| Formulation 12 | L-methionine, | 8 | 4 | histidine |
| | 0.2% w/w | | | |

In another embodiment, liquid compositions can be prepared with 100 U/mL botulinum toxin, a non-cross-linked hyaluronic acid or salt thereof, 8 w/w % trehalose, and 4 w/w % poloxamer P188 in histidine buffer at pH 6.0. Each formulation has a different antioxidant or a combination of antioxidants, as set forth in Table 5 below. The antioxidants tested included NAC, L-methionine, L-tryptophan, L-glutathione, sodium sulfite, propyl gallate, and EDTA sodium salt.

TABLE 5

| | Antioxidant² | | | | | | |
|---|---|---|---|---|---|---|---|
| | NAC | Met | TRP | GSH | NaSul | PrpGal | EDTA |
| Formulation No. ¹ | % | % | % | % | % | % | % |
| Formulation 20 | | 0.2 | | | | | |
| Formulation 21 | | | 0.2 | | | | |
| Formulation 22 | | | | 0.2 | | | |
| Formulation 23 | | | | | 0.2 | | |
| Formulation 24 | | | | | | 0.2 | |
| Formulation 25 | 0.2 | | 0.2 | | | | |
| Formulation 26 | 0.2 | | | | | | 0.03 |
| Formulation 27 | 0.2 | | 0.2 | | | | 0.03 |
| Formulation 28 | | | 0.2 | 0.2 | | | |

¹ Each formulation contains 100 U/mL botulinum toxin, 0.2 to 10 w/w % a non-cross-linked hyaluronic acid or salt thereof, 8 w/w % trehalose, and 4 w/w % poloxamer P188 in 20 mM histidine buffer, pH 6.0 and the specified antioxidant.
²NAC = N-acetyl-L-cysteine; Met = L-methionine; TRP = L-tryptophan; GSH = L-glutathione; NaSul = sodium sulfite; PrpGal = propyl gallate; EDTA = ethylene diamine tetraacetic acid, sodium salt.

Accordingly, compositions in liquid or solution form comprising a Clostridial toxin active ingredient; a non-cross-linked hyaluronic acid or salt thereof; a disaccharide; a surfactant; and an antioxidant selected from methionine, NAC, EDTA, EGTA, DTPA, analogues thereof, and combinations thereof, are contemplated. In a further embodiment, the composition is in liquid or solution form comprising a Clostridial toxin active ingredient; a non-cross-linked hyaluronic acid or salt thereof; a disaccharide; a surfactant; and an antioxidant selected from (i) methionine, and (ii) NAC and a chelating agent selected from EDTA, EGTA, DTPA, and analogues thereof.

In another embodiment, a liquid composition comprises a Clostridial toxin active ingredient; a non-cross-linked hyaluronic acid or salt thereof; a tonicity agent; a surfactant; and an antioxidant selected from the group consisting of a sacrificial antioxidant, a chelating agent antioxidant, a chain terminator antioxidant, and combinations thereof.

In another embodiment, liquid compositions can be prepared with 100 U/mL botulinum toxin, a non-cross-linked hyaluronic acid or salt thereof, 4 w/w % poloxamer P188, 0.2 w/w % methionine, and either 8 w/w % trehalose or sucrose, in histidine buffer at pH 6.0, as shown in Table 6.

TABLE 6
Liquid formulations

| Formulation No.* | Disaccharide |
|---|---|
| Formulation 30 | trehalose (8 w/w %) |
| Formulation 31 | sucrose (8 w/w %) |

*Each formulation contained 100 U/mL botulinum toxin, 0.2 to 10 w/w % non-cross-linked hyaluronic acid or salt thereof, 4 w/w % poloxamer P188 and 0.2 w/w % methionine in histidine buffer.

In one embodiment, a liquid composition comprised of a Clostridial toxin active ingredient, such as a botulinum toxin; a non-cross-linked hyaluronic acid or salt thereof; trehalose; a poloxamer surfactant; and methionine is contemplated.

In another embodiment, liquid compositions comprising a Clostridial toxin active ingredient, a non-cross-linked hyaluronic acid or salt thereof, poloxamer P188 (4 w/w %) or polysorbate (TWEEN® 20, 0.04 w/w %), trehalose (8% w/w), and methionine (0.2 w/w %), in 20 mM histidine buffer at pH 6.0, can be prepared (Table 7). The composition with poloxamer P188 is identified as Formulation No. 30 and the composition with polysorbate is identified as Formulation No. 32.

TABLE 7
Liquid formulations

| Formulation No.* | Surfactant |
|---|---|
| Formulation 30 | poloxamer P188 (4 w/w %) |
| Formulation 32 | polysorbate (TWEEN ® 20, 0.04 w/w %) |

*Each formulation contains 100 U/mL botulinum toxin, 0.2-10 w/w % non- cross-linked hyaluronic acid or salt thereof, 8 w/w % trehalose, and 0.2 w/w % methionine in histidine buffer.

In one embodiment, a liquid composition comprised of a Clostridial toxin active ingredient, such as a botulinum toxin; a non-cross-linked hyaluronic acid or salt thereof; trehalose; a poloxamer surfactant; and methionine is provided.

In another embodiment, liquid compositions can be prepared with botulinum toxin as a model Clostridial toxin active ingredient and a non-cross-linked hyaluronic acid or salt thereof. The compositions can be prepared with or without poloxamer surfactant, with or without trehalose, and with or without methionine. Details of the compositions are given in Table 8.

TABLE 8
Liquid formulations

Composition Components*

| | Toxin | Treha- | Poloxamer | Methi- |
|---|---|---|---|---|
| Formulation No. | U/mL | lose % | P188 % | onine % |
| Formulation 33 | 100 | 8 | 4 | 0 |
| Formulation 34 | 150 | 0 | 0 | 0.2 |
| Formulation 35 | 150 | 8 | 0 | 0.2 |
| Formulation 36 | 100 | 0 | 4 | 0.2 |
| Formulation 37 | 100 | 8 | 4 | 0.2 |

*Each formulation is in 20 mM histidine buffer at pH 6.0, and the concentration of a non-cross-linked hyaluronic acid or salt thereof is 0.2 to 10 w/w %.

In one embodiment, a liquid composition comprised of a Clostridial toxin active ingredient, such as a botulinum toxin; a non-cross-linked hyaluronic acid or salt thereof; a poloxamer surfactant; and an antioxidant such as methionine is contemplated. In another embodiment, a disaccharide is also included.

In another embodiment, liquid compositions can be prepared with botulinum toxin as a model Clostridial toxin active ingredient and a non-cross-linked hyaluronic acid or salt thereof. The compositions can be prepared with a poloxamer surfactant or with a polysorbate surfactant, with or without a disaccharide. All formulations comprise methionine. Details of the compositions are given in Table 9.

TABLE 9
Liquid formulations

| | Composition Components¹ | | | | | | |
|---|---|---|---|---|---|---|---|
| | Treh | Suc | NaCl | P188 | TWEEN ® 20 | Met | Buffer² |
| Formulation No. | % | % | % | % | % | % | or water |
| Formulation 31 | | 8 | | 4 | | 0.2 | buffer |
| Formulation 38 | | 8 | | 4 | | 0.2 | water |
| Formulation 39 | | | 0.9 | 4 | | 0.2 | buffer |
| Formulation 40 | 8 | | | | 0.04 | 0.2 | buffer |

¹All formulations comprise 100 U/mL botulinum toxin; 0.2 to 10 w/w % non-cross-linked hyaluronic acid or salt thereof; Treh = trehalose; Suc = sucrose; P-188 = poloxamer P188; TWEEN ® 20 = polysorbate; Met = L-methionine;
²Buffer = 20 mM His, pH 6.0.

In one embodiment, a liquid composition comprised of a Clostridial toxin active ingredient, such as a botulinum toxin; a non-cross-linked hyaluronic acid or salt thereof; a poloxamer or polysorbate surfactant; trehalose or sucrose; and an antioxidant such as methionine is contemplated.

In another embodiment, liquid compositions with no tonicity agent were prepared. Botulinum toxin was used as a model Clostridial toxin active ingredient. The compositions were prepared with either a poloxamer surfactant or with a polysorbate surfactant, and with methionine as the antioxidant. Details of the compositions are given in Table 10.

TABLE 10
Liquid formulations

| | Composition Components* | | | |
|---|---|---|---|---|
| | Toxin | | Polysorbate | Methi- |
| Formulation No. | U/mL | P188 % | (TWEEN ®20) % | onine % |
| Formulation 41 | 100 | 0 | 0.04 | 0.2 |
| Formulation 42 | 100 | 4 | 0 | 0.2 |

*Both formulations are in 20 mM histidine buffer at pH 6.0 and further contain 0.2 to 10 w/w % non-cross-linked hyaluronic acid or salt thereof. P-188 = poloxamer P188; Met = L-methionine

In another embodiment, liquid compositions can be prepared comprising a Clostridial toxin active ingredient; a non-cross-linked hyaluronic acid or salt thereof; poloxamer P188 (4% w/w or 0.6% w/w); trehalose (2% w/w or 8% w/w); and an antioxidant—(i) EDTA and NAC (0.03% w/w and 0.2% w/w, respectively), or (ii) methionine (0.2% w/w); in 20 mM histidine buffer at pH 6.0. Each formulation had between 30-200 U botulinum toxin per vial. A summary of the compositions is set forth in Table 11.

TABLE 11
Liquid formulations

| | Tonicity Agent | | Surfactant | | | | |
|---|---|---|---|---|---|---|---|
| | Trehalose | | Poloxamer | Antioxidant | | | |
| Toxin | or Sucrose | NaCl | P188 | Methionine | EDTA | NAC | |
| U/mL | wt % | wt % | wt % | wt % | wt % | wt % | Buffer |
| 30-200 | 8 | | 4 | 0.2 | | | 20 mM His, |
| | | | | | | | pH 6.0 |
| 30-200 | 8 | | 0.6 | 0.2 | | | 20 mM His, |
| | | | | | | | pH 6.0 |
| 30-200 | 2 | 0.6 | 4 | 0.2 | | | 20 mM His, |
| | | | | | | | pH 6.0 |
| 30-200 | 8 | | 4 | | 0.03 | 0.2 | 20 mM His, |
| | | | | | | | pH 6.0 |
| 30-200 | 8 | | 4 | 0.2 | 0.03 | | |

All formulations further comprise 0.2 to 10 w/w % non-cross-linked hyaluronic acid or salt thereof.

Accordingly, in one embodiment, a liquid pharmaceutical composition is contemplated. The composition comprises a Clostridial toxin active ingredient; a non-cross-linked hyaluronic acid or salt thereof; a tonicity agent selected from trehalose, sucrose, sodium chloride, mannitol, sorbitol, glucose and combinations thereof; a surfactant selected from a poloxamer, a polysorbate and combinations thereof; and an antioxidant selected from methionine, NAC, EDTA, EGTA, DTPA, analogues thereof, and combinations thereof. In one embodiment, the composition excludes albumin, and in another embodiment, when the antioxidant is methionine the composition excludes a polysorbate.

In another embodiment, a liquid pharmaceutical composition is contemplated. The composition comprises a Clostridial toxin active ingredient; a non-cross-linked hyaluronic acid or salt thereof; a surfactant selected from a poloxamer, a polysorbate and combinations thereof; and an antioxidant selected from methionine, NAC, EDTA, EGTA, DTPA, analogues thereof, and combinations thereof. In one embodiment, the composition excludes albumin. In one embodiment, the composition excludes a tonicity agent.

In another embodiment, a liquid pharmaceutical composition is contemplated. The composition comprises a Clostridial toxin active ingredient; a non-cross-linked hyaluronic acid or salt thereof; a tonicity agent selected from trehalose, sucrose, sodium chloride, mannitol, sorbitol, glucose, and combinations thereof; a poloxamer; and methionine. In one embodiment, the composition excludes albumin.

In another embodiment, a liquid pharmaceutical composition is contemplated. The composition comprises a Clostridial toxin active ingredient; a non-cross-linked hyaluronic acid or salt thereof; a disaccharide tonicity agent in an amount between 1-15 wt %; a poloxamer in an amount between 0.5-8 wt %; and an antioxidant in an amount between 0.05-5 wt %. In another embodiment, a composition comprises a Clostridial toxin active ingredient; a non-cross-linked hyaluronic acid or salt thereof; trehalose in an amount between 1-15 wt %; a poloxamer in an amount between 0.5-8 wt %; and methionine in an amount between 0.05-5 wt %. In one embodiment, the composition excludes albumin. In another embodiment, a liquid pharmaceutical composition comprises a Clostridial toxin active ingredient; a non-cross-linked hyaluronic acid or salt thereof; trehalose in an amount between 2-15 wt % or 1-10 wt %; a poloxamer in an amount between 0.5-8 wt %; and methionine in an amount between 0.05-5 wt %. In one embodiment, the composition excludes albumin.

In another embodiment, a liquid pharmaceutical composition is contemplated. The composition comprises a botulinum toxin; a non-cross-linked hyaluronic acid or salt thereof; a tonicity agent selected from trehalose, sucrose, sodium chloride, mannitol, sorbitol, glucose, and combinations thereof; a poloxamer; and an antioxidant selected from methionine, NAC, EDTA, EGTA, DTPA, analogues thereof, and combinations thereof. In one embodiment, the composition excludes albumin.

In any of the foregoing embodiments, it is contemplated that the composition is not, in some embodiments, an emulsion and/or excludes nanoparticles comprising an amphiphilic entity.

Lyophilized compositions can be prepared. The compositions that were prepared comprised botulinum toxin as a model Clostridial toxin; a non-cross-linked hyaluronic acid or salt thereof; a disaccharide; a surfactant; and methionine as an antioxidant. The formulations can be lyophilized and stored for about two weeks at −20° C. or at 40° C. The solid compositions are shown in Table 12.

TABLE 12
Lyophilized formulations

| | Composition Components* | | | | |
|---|---|---|---|---|---|
| | Treha- | Sucrose | TWEEN ® | Poloxamer | Methi- |
| Formulation No. | lose % | % | 20% | P188% | onine % |
| Formulation 43 | 8 | 0 | 0 | 4 | 0.2 |
| Formulation 44 | 8 | 0 | 0.04 | 0 | 0.2 |
| Formulation 45 | 0 | 8 | 0 | 4 | 0.2 |
| Formulation 46 | 0 | 8 | 0.04 | 0 | 0.2 |

*Each formulation comprises 200 U/vial BoNT/A and 0.2 to 10 w/w % non-crosslinked hyaluronic acid or salt thereof and is in 20 mM histidine buffer at pH 6.0.

In one embodiment, a lyophilized composition is provided, where the composition comprises a Clostridial toxin active ingredient, a non-cross-linked hyaluronic acid or salt thereof, a poloxamer, methionine, and trehalose.

In one embodiment, a lyophilized composition is provided, where the composition comprises a Clostridial toxin active ingredient, a non-cross-linked hyaluronic acid or salt thereof; a poloxamer, methionine, and trehalose.

In one embodiment, a solid or lyophilized pharmaceutical composition is contemplated. The composition comprises a Clostridial toxin active ingredient; a non-cross-linked hyaluronic acid or salt thereof; a tonicity agent and/or lyoprotector selected from trehalose, sucrose, mannitol, sorbitol, glucose, and combinations thereof; a surfactant selected from a poloxamer, a polysorbate and combinations thereof; and an antioxidant selected from methionine, NAC, EDTA, EGTA, DTPA, analogues thereof, and combinations thereof. In some embodiments, the solid composition comprises a lyoprotector. In some embodiments, the lyoprotector includes sucrose, trehalose, mannitol, sorbitol, glucose, or combinations thereof. In certain embodiments, the lyophilized composition is reconstituted with a tonicity agent selected from trehalose, sucrose, sodium chloride, mannitol, sorbitol, glucose, and combinations thereof. In at least one embodiment, the lyophilized composition is reconstituted with a reconstitution vehicle comprising NaCl prior to administration to a patient. In at least one embodiment, NaCl is present in an amount of 0.9% (w/w) in the reconstitution vehicle. In other embodiments, KCl is included in the composition, in an amount suitable for tonicity adjustment. In one embodiment, the composition excludes albumin, a hydroxyalkylstarch, glutamic acid, glutamine, aspartic acid, asparagine, a polyalcohol, glycine, and/or polyvinylpyrrolidone.

In another embodiment, a solid or lyophilized pharmaceutical composition is contemplated. The composition comprises a Clostridial toxin active ingredient; a non-cross-linked hyaluronic acid or salt thereof; trehalose or sucrose; a poloxamer; and methionine. In one embodiment, the composition excludes albumin, a hydroxyalkylstarch, glutamic acid, glutamine, aspartic acid, asparagine, a polyalcohol, glycine, and/or polyvinylpyrrolidone. In one embodiment, the lyophilized pharmaceutical composition comprises botulinum toxin as the Clostridial toxin active ingredient, a non-cross-linked hyaluronic acid or salt thereof; trehalose, a poloxamer, and methionine.

In another embodiment, a solid or lyophilized pharmaceutical composition is contemplated. The composition comprises a Clostridial toxin active ingredient; a non-cross-linked hyaluronic acid or salt thereof; trehalose in an amount between 1-15 wt %; a poloxamer in an amount between 0.5-8 wt %; and methionine in an amount between 0.05-5 wt %. In another embodiment, the composition comprises a Clostridial toxin active ingredient, such as botulinum toxin; 8 wt % trehalose; 4 wt % poloxamer; and 0.2 wt % methionine. In one embodiment, the botulinum toxin is present in an amount of about 200 units. In another embodiment, the botulinum toxin is present in an amount of about 50 units. In one embodiment, the composition excludes albumin, a hydroxyalkylstarch, glutamic acid, glutamine, aspartic acid, asparagine, a polyalcohol, glycine, and/or polyvinylpyrrolidone.

In another embodiment, a solid or lyophilized pharmaceutical composition is contemplated. The composition comprises a botulinum toxin; a non-cross-linked hyaluronic acid or salt thereof; a disaccharide; a poloxamer; and an antioxidant selected from methionine, NAC, EDTA, EGTA, DTPA, analogues thereof, and combinations thereof. In one embodiment, the composition excludes albumin, a hydroxyalkylstarch, glutamic acid, glutamine, aspartic acid, asparagine, a polyalcohol, glycine, and/or polyvinylpyrrolidone.

In another embodiment, a lyophilized composition comprises a Clostridial toxin active ingredient; a non-cross-linked hyaluronic acid or salt thereof; a tonicity agent and/or a lyoprotector; a surfactant; and an antioxidant selected from the group consisting of a sacrificial antioxidant, a chelating agent antioxidant, a chain terminator antioxidant, and combinations thereof. In some embodiments, the lyophilized Clostridial pharmaceutical composition comprises a lyoprotector. In some embodiments, the lyoprotector includes sucrose, trehalose, mannitol, sorbitol, glucose, or combinations thereof.

In any of the foregoing embodiments of solid or liquid compositions, it is contemplated that one or more, in any combination, of these ingredients are excluded: polyvinylpyrrolidone, diblock copolymers of polypropylene glycol and polyethylene glycol, and/or a polyalcohol such as inositol, lactilol, isomalt, xylitol, or erythritol.

In one embodiment, a liquid composition as disclosed herein can be prepared by reconstituting with a reconstitution vehicle comprising NaCl or KCl, prior to administration to a patient, a non-cross-linked sodium hyaluronate powder and a lyophilized formulations comprising no non-crosslinked hyaluronic acid or salt thereof, such as any of the lyophilized formulations disclosed above but without the non-crosslinked hyaluronic acid or salt thereof. In at least one embodiment, NaCl or KCl is present in an amount of 0.9% (w/w) in the reconstitution vehicle.

In any of the foregoing embodiments of solid or liquid compositions, it is contemplated that the weight average molecular weight of non-crosslinked hyaluronic acid or salt thereof can be from about 450 kDa to 2.0 MDa, such as from 450 kDa to 1.6 MDa, such as about 1.58 MDa.

In any of the foregoing embodiments of solid or liquid compositions, it is contemplated that the concentration of a non-cross-linked hyaluronic acid or salt thereof is from about 0.2 to 10 wt %, 0.2-9 wt %, 0.2-8 wt %, 0.2-6 wt %, 0.3-10 wt %, 0.3-9 wt %, 0.3-6 wt %, 0.3-5 wt %, 0.4 to 5 wt %, 0.5-5 wt %, 0.5-2.5 wt %, 0.5-2 wt %, 0.75-2 wt %, 1-2 wt %. In other embodiments, the concentration of a non-cross-linked hyaluronic acid or salt thereof is about, at least about, or between about any of the following: 0.4, 0.5, 0.6, 0.7, 0.8, 0.9, 1.0, 1.1, 1.2, 1.3, 1.4, 1.5, 2, 2.5, 3, 3.5, 4, 4.5, 5, 5.5, or 6 wt %.

In any of the foregoing aspects/embodiments of liquid compositions, it is contemplated that the viscosity of the pharmaceutical composition is from about 0.01 Pa-s to about 0.2 Pa-s (about 10 cps to about 200 cps) at 25° C., at a shear rate of 0.1/second. In one embodiment, the composition has a viscosity of less than about 500 Pa-s at a shear rate of 0.1/sec at 25° C. In other embodiments, the composition has a viscosity of less than about 475 Pa-s, 450 Pa-s, 400 Pa-s, 350 Pa-s, 300 Pa-s, 250 Pa-s, 225 Pa-s, 200 Pa-s, 175 Pa-s, 150 Pa-s, 125 Pa-s or 110 Pa-s, at 25° C., at a shear rate of 0.1/second. In other embodiments, the composition has a viscosity of between about 1-500 Pa-s, 2-500 Pa-s, 5-500 Pa-s, 10-500 Pa-s, 20-500 Pa-s, 30-500 Pa-s, 50-500 Pa-s, 75-500 Pa-s, 80-500 Pa-s, 90-500 Pa-s, 10-400 Pa-s, 20-400 Pa-s, 30-400 Pa-s, 50-400 Pa-s, 75-400 Pa-s, 80-400 Pa-s, 90-400 Pa-s, 10-300 Pa-s, 20-300 Pa-s, 30-300 Pa-s, 50-300 Pa-s, 75-300 Pa-s, 80-300 Pa-s, 90-300 Pa-s, 10-250 Pa-s, 20-250 Pa-s, 30-250 Pa-s, 50-250 Pa-s, 75-250 Pa-s, 80-250 Pa-s, 90-250 Pa-s, 10-200 Pa-s, 20-200 Pa-s, 30-200 Pa-s, 50-200 Pa-s, 75-200 Pa-s, 80-200 Pa-s, 90-200 Pa-s, 10-150 Pa-s, 20-150 Pa-s, 30-150 Pa-s, 50-150 Pa-s, 75-150 Pa-s, 80-150 Pa-s, 90-150 Pa-s, at 25° C., at a shear rate of 0.1/second. In other embodiments, the viscosity is from about, at least about, or between about any of the following: 10 Pa-s., 15 Pa-s, 20 Pa-s, 25 Pa-s, 30 Pa-s, 40 Pa-s, 45 Pa-s, 50 Pa-s, 55 Pa-s, 60 Pa-s, 65 Pa-s, 70 Pa-s, 75 Pa-s, 80 Pa-s, 85 Pa-s, 90 Pa-s, 95 Pa-s, 100 Pa-s, 110 Pa-s, 125 Pa-s, 150 Pa-s, 175 Pa-s, 200 Pa-s, 225 Pa-s, 250 Pa-s, 275 Pa-s, 300 Pa-s, 325 Pa-s, 350 Pa-s, 375 Pa-s, 400 Pa-s, 425 Pa-s, 450 Pa-s, 475 Pa-s, 500 Pa-s, 550 Pa-s, 600 Pa-s, 650 Pa-s, 700 Pa-s, 800 Pa-s, 900 Pa-s, 1000 Pa-s, at 25° C., at a shear rate of 0.1/second, measured as described in Example 1.

In any of the foregoing aspects/embodiments of liquid compositions, it is contemplated that the composition can comprise up to 10 Units, such as up to 6 Units, of Clostridial toxin per milligram of a non-cross-linked hyaluronic acid or salt thereof.

In any of the foregoing aspects/embodiments of liquid compositions, it is contemplated that the concentration of the Clostridial toxin can be from about 0.2 to 2.5 ng/mL, such as about, between above, or at least about any of the following: 0.2, 0.4, 0.6, 0.8, 1.0, 1.2, 1.4, 1.5, 1.6, 1.8, 2.0, 2.1, 2.2, 2.4, 2.5, 3.0, 4.0, 4.5, 5.0, 5.5, 6.0. 6.5, 7.0, 7.5, 8.0, 8.5, 9.0, 9.5 or 10, 12, or 15 ng/mL. In another embodiment, it is contemplated that the concentration of the Clostridial toxin in any of the solid or liquid compositions described herein can be from about 10 U/mL to about 200 U/mL, such as about, between above, or at least about any of the following: 10, 15, 20, 25, 30, 35, 40, 45, 50, 60, 75, 90, 100, 110, 125, 135, 150, 160, 175, 190, or 200 U/mL.

In any of the foregoing embodiments of solid or liquid compositions, it is contemplated that the composition can be prepared by mixing the Clostridial toxin active ingredient with a composition comprising a non-cross-linked hyaluronic acid or salt thereof and diluent(s). Accordingly, a composition, comprising a non-cross-linked hyaluronic acid or salt thereof, which is otherwise identical to any of the solid or liquid compositions disclosed above but for comprising no Clostridial toxin active ingredient, is also provided.

### Pharmaceutical Composition Components

The present pharmaceutical compositions include a Clostridial toxin or a Clostridial toxin active ingredient and a non-cross-linked hyaluronic acid or salt thereof, such as from about 450 kDa to 2.0 MDa, such as from 450 kDa to 1.6 MDa, and about 1.4-1.6 MDa. A skilled artisan will appreciate that the description herein refers to a Clostridial toxin active ingredient, however, a Clostridial toxin may also be used in the compositions described herein. Accordingly, the term Clostridial toxin active ingredient will be used; however it should be understood that a Clostridial toxin is equally contemplated. In one embodiment, a therapeutically effective concentration of a Clostridial toxin active ingredient is present in the composition. In one embodiment, the Clostridial toxin active ingredient reduces a symptom associated with the aliment being treated by, e.g., at least 10%, at least 20%, at least 30%, at least 40%, at least 50%, at least 60%, at least 70%, at least 80%, at least 90% or at least 100%. In other aspects of this embodiment, a therapeutically effective concentration of a Clostridial toxin active ingredient reduces a symptom associated with the aliment being treated by, e.g., at most 10%, at most 20%, at most 30%, at most 40%, at most 50%, at most 60%, at most 70%, at most 80%, at most 90% or at most 100%.

It is envisioned that any amount of Clostridial toxin active ingredient can be added in formulating a Clostridial toxin active ingredient pharmaceutical compositions disclosed herein, with the proviso that a therapeutically effective amount of Clostridial toxin active ingredient is recoverable. In aspects of this embodiment, the amount of Clostridial toxin active ingredient added to the formulation is at least 0.1 U/ml, at least 1.0 U/ml, at least 10 U/ml, at least 50 U/ml, at least 100 U/ml, at least 200 U/ml, or at least 1000 U/ml. In other aspects of this embodiment, the amount of Clostridial toxin active ingredient added to the formulation is at most 0.1 U/ml, at most 1.0 U/ml, at most 10 U/ml, at most 50 U/ml, at most 100 U/ml, at most 200 U/ml, or at most 1000 U/ml. In yet other aspects of this embodiment, the amount of Clostridial toxin active ingredient added to the formulation is from about 0.1 U/ml to about 1000 U/ml, or about 1.0 U/ml to about 1000 U/ml. In still other aspects of this embodiment, the amount of Clostridial toxin active ingredient added to the formulation is from about 0.001 U/ml to about 100 U/ml, about 0.01 U/ml to about 100 U/ml, about 0.1 U/ml to about 100 U/ml, or about 1.0 U/ml to about 100 U/ml.

In other aspects of this embodiment, the amount of Clostridial toxin active ingredient added to the formulation is at least 1.0 pg, at least 10 pg, at least 100 pg, at least 1.0 ng, at least 10 ng, at least 100 ng, at least 1.0 μg, at least 10 μg, at least 100 μg, or at least 1.0 mg. In still other aspects of this embodiment, the amount of Clostridial toxin active ingredient added to the formulation is at most 1.0 pg, at most 10 pg, at most 100 pg, at most 1.0 ng, at most 10 ng, at most 100 ng, at most 1.0 μg, at most 10 μg, at most 100 μg, or at most 1.0 mg. In still other aspects of this embodiment, the amount of Clostridial toxin active ingredient added to the formulation is about 1.0 pg to about 10 μg, about 10 pg to about 10 μg, about 100 pg to about 10 μg, about 1.0 ng to about 10 μg, about 10 ng to about 10 μg, or about 100 ng to about 10 μg. In still other aspects of this embodiment, the amount of Clostridial toxin active ingredient added to the formulation is about 1.0 pg to about 1.0 μg, about 10 pg to about 1.0 μg, about 100 pg to about 1.0 μg, about 1.0 ng to about 1.0 μg, about 10 ng to about 1.0 μg, or about 100 ng to about 1.0 μg. In further aspects of this embodiment, the amount of Clostridial toxin active ingredient added to the formulation is about 1.0 pg to about 5.0 μg, about 10 pg to about 5.0 μg, about 100 pg to about 5.0 μg, about 1.0 ng to about 5.0 μg, about 10 ng to about 5.0 μg, or about 100 ng to about 5.0 μg. In further aspects of this embodiment, the amount of Clostridial toxin active ingredient added to the formulation is about 1.0 pg to about 10 μg, about 10 pg to about 10 μg, about 100 pg to about 10 μg, about 1.0 ng to about 10 μg, about 10 ng to about 10 μg, or about 100 ng to about 10 μg.

In aspects of this embodiment, a Clostridial toxin pharmaceutical composition comprises a BoNT/A, a BoNT/B, a BoNT/C₁, a BoNT/D, a BoNT/E, a BoNT/F, a BoNT/G, a mosaic BoNT such as for example BoNT/DC, a TeNT, a BaNT, or a BuNT. In another embodiment, a Clostridial toxin pharmaceutical composition comprises a Clostridial toxin variant as the Clostridial toxin. In aspects of this embodiment, a Clostridial toxin pharmaceutical composition comprises naturally-occurring Clostridial toxin active ingredient variant or a non-naturally-occurring Clostridial toxin variant. In other aspects of this embodiment, a Clostridial toxin pharmaceutical composition comprises a BoNT/A variant, a BoNT/B variant, a BoNT/C₁ variant, a BoNT/D variant, a BoNT/E variant, a BoNT/F variant, a BoNT/G variant, a TeNT variant, a BaNT variant, or a BuNT variant, where the variant is either a naturally-occurring variant or a non-naturally-occurring variant.

Aspects of the present pharmaceutical compositions provide, in part, a Clostridial toxin complex as a Clostridial toxin active ingredient. As used herein, the term “Clostridial toxin complex” refers to a complex comprising a Clostridial toxin and associated NAPs, such as, e.g., a Botulinum toxin complex, a Tetanus toxin complex, a Baratii toxin complex, and a Butyricum toxin complex. Non-limiting examples of Clostridial toxin complexes include those produced by a Clostridium botulinum, such as, e.g., a 900-kDa BoNT/A complex, a 500-kDa BoNT/A complex, a 300-kDa BoNT/A complex, a 500-kDa BoNT/B complex, a 500-kDa BoNT/C₁ complex, a 500-kDa BoNT/D complex, a 300-kDa BoNT/D complex, a 300-kDa BoNT/E complex, and a 300-kDa BoNT/F complex. Clostridial toxin complexes can be purified using the methods described in Schantz, supra, (1992); Hui Xiang et al., Animal Product Free System and Process for Purifying a Botulinum Toxin, U.S. Pat. No. 7,354,740, each of which is hereby incorporated by reference in its entirety. Clostridial toxin complexes can be obtained from, e.g., List Biological Laboratories, Inc. (Campbell, Calif.), the Centre for Applied Microbiology and Research (Porton Down, U.K), Wako (Osaka, Japan), and Sigma Chemicals (St Louis, Mo.).

The pharmaceutical compositions comprise, in most embodiments, a non-cross-linked hyaluronic acid or salt thereof. Hyaluronic acid (also called hyaluronan) is a naturally occurring polysaccharide found in joints, connective tissue and the eye. Hyaluronic acid is a glycosaminoglycan (a mucopolysaccharide) which is a long unbranched polysaccharide composed of repeating dimeric units of glucuronic acid and N acetyl glucosamine. U.S. Pat. Nos. 4,636,524; 4,713,448; 5,099,013, and 5,143,724 disclose particular hyaluronic acids and methods for making them. Salt forms of hyaluronic acid include, for example, sodium, potassium, calcium forms of hyaluronic acid (e.g, sodium hyaluronate, potassium hyaluronate, calcium hyaluronate).

In the studies described herein non-cross-linked hyaluronic acid with the following properties were used.

| | C of A | | | | Viscosity² |
|---|---|---|---|---|---|
| Nomen- | MW/intrinsic | | | | (Pa-s) @ 0.1/sec |
| clature | viscosity | | MW¹ | | and HA conc. |
| LMW | 0.76 | m³/kg | 401 | kDa | 37 @ 4.3% HA |
| HMW | 3.03 | m³/kg | 2453 | kDa | 140 @ 1.35% HA |
| 700 | 738 | kDa | 683 | kDa | 40 to 260 @ 2.3% to 4% HA |
| 1500 | 1580 | kDa | 1316 | kDa | 40 to 260 @ 1.2% to 2% HA |
| 1500 | 1560 | kDa | 1568 | kDa | 92 to 560 @ 1.2% to 2% HA |
| 2000 | 2670 | kDa | 2366 | kDa | 30 @ 0.5% HA |

¹weight average molecular weight, measured using size exclusion chromatography with DAWN HELEOS multi-angle light scattering and Optilab rEX refractive index
²measured using an Anton Parr rheometer (Example 1)

The weight average molecular weight of the non-cross-linked hyaluronic acid or salt thereof for use in the compositions described herein is generally less than about 2.5 MDa or less than about 2.4 MDa. In one embodiment, the non-cross-linked hyaluronic acid, or salt thereof, has a weight average molecular weight of between about 250 kDa and about 2.4 MDa. In another embodiment, the non-cross-linked hyaluronic acid, or salt thereof, has a weight average molecular weight of between about 4.6 MDa and about 8 MDa. In still other embodiments, the non-cross-linked hyaluronic acid, or salt thereof, has a weight average molecular weight of between about 300 kDa-2.0 MDa, 300 kDa-1.5 MDa, 300 kDa-1.25 MDa, 300 kDa-1.0 MDa, 300 kDa-9000 kDa, 300 kDa-8000 kDa, 300 kDa-7000 kDa, 300 kDa-6000 kDa, 300 kDa-5000 kDa, 300 kDa-4000 kDa, 300 kDa-3000 kDa, 300 kDa-2000 kDa, 400 kDa-2.0 MDa, 400 kDa-1.5 MDa, 400 kDa-1.25 MDa, 400 kDa-1.0 MDa, 400 kDa-9000 kDa, 400 kDa-8000 kDa, 400 kDa-7000 kDa, 400 kDa-6000 kDa, 400 kDa-5000 kDa, 400 kDa-4000 kDa, 400 kDa-3000 kDa, 400 kDa-2000 kDa, 500 kDa-2.0 MDa, 500 kDa-1.5 MDa, 500 kDa-1.25 MDa, 500 kDa-1.0 MDa, 500 kDa-9000 kDa, 500 kDa-8000 kDa, 500 kDa-7000 kDa, 500 kDa-6000 kDa, 500 kDa-5000 kDa, 500 kDa-4000 kDa, 500 kDa-3000 kDa, 500 kDa-2000 kDa, 650-2000 kDa, 650-1800 kDa, 1000-2000 kDa, 1200-1800 kDa, 1300-1700 kDa, 1400-1600 kDa. In one embodiment, the non-cross-linked hyaluronic acid or salt thereof has a weight average molecular weight of about 1500 kDa or 1580 kDa.

In some embodiments, the non-cross-linked hyaluronic acid or salt thereof for use in the compositions described herein have a polydispersity index, which corresponds to the weight average molecular weight (MW) over the number average molecular weight (Mn), of between about 1-3, between about 1-2, between about 1.05-1.75, 1.05-1.60, 1.05-1.50, 1.05-1.40, 1.05-1.35, or 1.05-1.25.

In some embodiments, the pharmaceutical compositions comprise a non-protein excipient. As used herein, the term “non-protein excipient” refers to any excipient that is not a polypeptide comprising at least fifteen amino acids. It is envisioned that any non-protein excipient is useful in formulating a Clostridial toxin active ingredient pharmaceutical compositions disclosed in the present specification, with the proviso that a therapeutically effective amount of the Clostridial toxin active ingredient is recovered using this non-protein excipient.

In some embodiments, the pharmaceutical compositions comprise a sugar. As used herein, the term “sugar” refers to a compound comprising one to 10 monosaccharide units, e.g., a monosaccharide, a disaccharide, a trisaccharide, and an oligosaccharide comprising four to ten monosaccharide units. It is envisioned that any sugar is useful in formulating a Clostridial toxin active ingredient pharmaceutical compositions disclosed in the present specification, with the proviso that a therapeutically effective amount of the Clostridial toxin active ingredient is recovered using this sugar. In some embodiments, for example in a lyophilized composition, the sugar can function as a lyoprotector. In some other embodiments, for example in a lyophilized formulation or in a liquid formulation, the sugar can function as a tonicity agent. Monosaccharides are polyhydroxy aldehydes or polyhydroxy ketones with three or more carbon atoms, including aldoses, dialdoses, aldoketoses, ketoses and diketoses, as well as cyclic forms, deoxy sugars and amino sugars, and their derivatives, provided that the parent monosaccharide has a (potential) carbonyl group. Monosacchrides include trioses, like glyceraldehyde and dihydroxyacetone; tetroses, like erythrose, erythrulose and threose; pentoses, like arabinose, lyxose, ribose, ribulose, xylose, xylulose; hexoses, like allose, altrose, fructose, fucose, galactose, glucose, gulose, idose, mannose, psicose, rhamnose, sorbose, tagatose, talose and trehalose; heptoses, like sedoheptulose and mannoheptulose; octooses, like octulose and 2-keto-3-deoxy-manno-octonate; nonoses like sialose; and decose. Oligosaccharides are compounds in which at least two monosaccharide units are joined by glycosidic linkages. According to the number of units, they are called disaccharides, trisaccharides, tetrasaccharides, pentasaccharides, hexoaccharides, heptoaccharides, octoaccharides, nonoaccharides, decoaccharides, etc. An oligosaccharide can be unbranched, branched or cyclic. Common disaccharides include, without limitation, sucrose, lactose, maltose, trehalose, cellobiose, gentiobiose, kojibiose, laminaribiose, mannobiose, melibiose, nigerose, rutinose, and xylobiose. Common trisaccharides include, without limitation, raffinose, acarbose, maltotriose, and melezitose. Other non-limiting examples of specific uses of sugar excipients can be found in, e.g., Ansel, supra, (1999); Gennaro, supra, (2000); Hardman, supra, (2001); and Rowe, supra, (2003), each of which is hereby incorporated by reference in its entirety.

In an embodiment, a Clostridial toxin active ingredient pharmaceutical composition comprises a sugar. In aspects of this embodiment, a Clostridial toxin active ingredient pharmaceutical composition comprises a monosaccharide. In other aspects of this embodiment, a Clostridial toxin active ingredient pharmaceutical composition comprises a disaccharide, a trisaccharide, a tetrasaccharide, a pentasaccharide, a hexoaccharide, a heptoaccharide, an octoaccharide, a nonoaccharide, or a decoaccharide. In yet other aspects of this embodiment, a Clostridial toxin active ingredient pharmaceutical composition comprises an oligosaccharide comprising two to ten monosaccharide units.

It is envisioned that any amount of sugar is useful in formulating a Clostridial toxin active ingredient pharmaceutical compositions disclosed in the present specification, with the proviso that a therapeutically effective amount of the Clostridial toxin active ingredient is recovered using this sugar amount. In aspects of this embodiment, the amount of sugar added to the formulation is about 0.1% (w/w), about 0.5% (w/w), about 1.0% (w/w), about 1.5% (w/w), about 2.0% (w/w), about 2.5% (w/w), about 3.0% (w/w), about 3.5% (w/w), about 4.0% (w/w), about 4.5% (w/w), about 5.0% (w/w), about 5.5% (w/w), about 6.0% (w/w), about 6.5% (w/w), about 7.0% (w/w), about 7.5% (w/w), about 8.0% (w/w), about 8.5% (w/w), about 9.0% (w/w), about 9.5% (w/w), about 10% (w/w), about 15% (w/w), about 20% (w/w), about 25% (w/w), about 30% (w/w), or about 35% (w/w). In other aspects of this embodiment, the amount of sugar added to the formulation is at least 0.1% (w/w), at least 0.5% (w/w), at least 1.0% (w/w), at least 1.5% (w/w), at least 2.0% (w/w), at least 2.5% (w/w), at least 3.0% (w/w), at least 3.5% (w/w), at least 4.0% (w/w), at least 4.5% (w/w), at least 5.0% (w/w), at least 5.5% (w/w), at least 6.0% (w/w), at least 6.5% (w/w), at least 7.0% (w/w), at least 7.5% (w/w), at least 8.0% (w/w), at least 8.5% (w/w), at least 9.0% (w/w), at least 9.5% (w/w), at least 10% (w/w), at least 15% (w/w), at least 20% (w/w), at least 25% (w/w), at least 30% (w/w), or at least 35% (w/w). In yet other aspects of this embodiment, the amount of sugar added to the formulation is at most 0.1% (w/w), at most 0.5% (w/w), at most 1.0% (w/w), at most 1.5% (w/w), at most 2.0% (w/w), at most 2.5% (w/w), at most 3.0% (w/w), at most 3.5% (w/w), at most 4.0% (w/w), at most 4.5% (w/w), at most 5.0% (w/w), at most 5.5% (w/w), at most 6.0% (w/w), at most 6.5% (w/w), at most 7.0% (w/w), at most 7.5% (w/w), at most 8.0% (w/w), at most 8.5% (w/w), at most 9.0% (w/w), at most 9.5% (w/w), at most 10% (w/w), at most 15% (w/w), at most 20% (w/w), at most 25% (w/w), at most 30% (w/w), or at most 35% (w/w).

In an embodiment, the present Clostridial toxin active ingredient pharmaceutical composition comprises a disaccharide. Common disaccharides include, without limitation, sucrose, lactose, maltose, trehalose, cellobiose, gentiobiose, kojibiose, laminaribiose, mannobiose, melibiose, nigerose, rutinose, and xylobiose. In aspects of this embodiment, the Clostridial toxin active ingredient pharmaceutical composition comprises sucrose. In one specific embodiment, the Clostridial toxin active ingredient pharmaceutical composition comprises trehalose. In aspects of this embodiment, the amount of disaccharide added to the formulation added to the formulation is about 0.1% (w/w), about 0.5% (w/w), about 1.0% (w/w), about 1.5% (w/w), about 2.0% (w/w), about 2.5% (w/w), about 3.0% (w/w), about 3.5% (w/w), about 4.0% (w/w), about 4.5% (w/w), about 5.0% (w/w), about 5.5% (w/w), about 6.0% (w/w), about 6.5% (w/w), about 7.0% (w/w), about 7.5% (w/w), about 8.0% (w/w), about 8.5% (w/w), about 9.0% (w/w), about 9.5% (w/w), about 10% (w/w), about 15% (w/w), about 20% (w/w), about 25% (w/w), about 30% (w/w), or about 35% (w/w).

Aspects of the present pharmaceutical compositions provide, in part, a surfactant. It is envisioned that any surfactant is useful in formulating a Clostridial toxin active ingredient pharmaceutical compositions disclosed in the present specification, with the proviso that a therapeutically effective amount of the Clostridial toxin active ingredient is recovered using this surfactant amount. Non-limiting examples of surfactants include polysorbates like polysorbate 20 (TWEEN® 20), polysorbate 40 (TWEEN® 40), polysorbate 60 (TWEEN® 60), polysorbate 61 (TWEEN® 61), polysorbate 65 (TWEEN® 65), polysorbate 80 (TWEEN® 80), and polysorbate 81 (TWEEN® 81); poloxamers (polyethylene-polypropylene copolymers), like Poloxamer 124 (PLURONIC® L44), Poloxamer 181 (PLURONIC® L61), Poloxamer 182 (PLURONIC® L62), Poloxamer 184 (PLURONIC® L64), Poloxamer 188 (PLURONIC® F68), Poloxamer 237 (PLURONIC® F87), Poloxamer 338 (PLURONIC® L108), Poloxamer 407 (PLURONIC® F127), polyoxyethyleneglycol dodecyl ethers, like BRIJ® 30, and BRIJ® 35; 2-dodecoxyethanol (LUBROL®-PX); polyoxyethylene octyl phenyl ether (TRITON® X-100); sodium dodecyl sulfate (SDS); solutol HS15; 3-[(3-Cholamidopropyl)dimethylammonio]-1-propanesulfonate (CHAPS); 3-[(3-Cholamidopropyl)dimethylammonio]-2-hydroxy-1-propanesulfonate (CHAPSO); sucrose monolaurate; and sodium cholate. Other non-limiting examples of surfactant excipients can be found in, e.g., Ansel, supra, (1999); Gennaro, supra, (2000); Hardman, supra, (2001); and Rowe, supra, (2003), each of which is hereby incorporated by reference in its entirety.

Thus in an embodiment, a Clostridial toxin active ingredient pharmaceutical composition comprises a surfactant. In aspects of this embodiment, a Clostridial toxin active ingredient pharmaceutical composition comprises a polysorbate, a poloxamer, a polyoxyethyleneglycol dodecyl ether, 2-dodecoxyethanol, polyoxyethylene octyl phenyl ether, sodium dodecyl sulfate, 3-[(3-Cholamidopropyl)dimethylammonio]-1-propanesulfonate, 3-[(3-Cholamidopropyl) dimethylammonio]-2-hydroxy-1-propanesulfonate, sucrose monolaurate; or sodium cholate.

It is envisioned that any amount of surfactant is useful in formulating a Clostridial toxin active ingredient pharmaceutical compositions disclosed in the present specification, with the proviso that a therapeutically effective amount of the Clostridial toxin active ingredient is recovered using this surfactant amount. In aspects of this embodiment, the amount of surfactant added to the formulation is about 0.01% (w/w), about 0.02% (w/w), about 0.03% (w/w), about 0.04% (w/w), about 0.05% (w/w), about 0.06% (w/w), about 0.07% (w/w), about 0.08% (w/w), about 0.09% (w/w), about 0.1% (w/w), about 0.5% (w/w), about 1.0% (w/w), about 1.5% (w/w), about 2.0% (w/w), about 2.5% (w/w), about 3.0% (w/w), about 3.5% (w/w), about 4.0% (w/w), about 4.5% (w/w), about 5.0% (w/w), about 5.5% (w/w), about 6.0% (w/w), about 6.5% (w/w), about 7.0% (w/w), about 7.5% (w/w), about 8.0% (w/w), about 8.5% (w/w), about 9.0% (w/w), about 9.5% (w/w), about 10% (w/w), about 15% (w/w), about 20% (w/w), about 25% (w/w), about 30% (w/w), or about 35% (w/w). In other aspects of this embodiment, the amount of surfactant added to the formulation is at least 0.01% (w/w), at least 0.02% (w/w), at least 0.03% (w/w), at least 0.04% (w/w), at least 0.05% (w/w), at least 0.06% (w/w), at least 0.07% (w/w), at least 0.08% (w/w), at least 0.09% (w/w), at least 0.1% (w/w), at least 0.5% (w/w), at least 1.0% (w/w), at least 1.5% (w/w), at least 2.0% (w/w), at least 2.5% (w/w), at least 3.0% (w/w), at least 3.5% (w/w), at least 4.0% (w/w), at least 4.5% (w/w), at least 5.0% (w/w), at least 5.5% (w/w), at least 6.0% (w/w), at least 6.5% (w/w), at least 7.0% (w/w), at least 7.5% (w/w), at least 8.0% (w/w), at least 8.5% (w/w), at least 9.0% (w/w), at least 9.5% (w/w), at least 10% (w/w), at least 15% (w/w), at least 20% (w/w), at least 25% (w/w), at least 30% (w/w), or at least 35% (w/w). In yet other aspects of this embodiment, the amount of surfactant added to the formulation is at most 0.01% (w/w), at most 0.02% (w/w), at most 0.03% (w/w), at most 0.04% (w/w), at most 0.05% (w/w), at most 0.06% (w/w), at most 0.07% (w/w), at most 0.08% (w/w), at most 0.09% (w/w), at most 0.1% (w/w), at most 0.5% (w/w), at most 1.0% (w/w), at most 1.5% (w/w), at most 2.0% (w/w), at most 2.5% (w/w), at most 3.0% (w/w), at most 3.5% (w/w), at most 4.0% (w/w), at most 4.5% (w/w), at most 5.0% (w/w), at most 5.5% (w/w), at most 6.0% (w/w), at most 6.5% (w/w), at most 7.0% (w/w), at most 7.5% (w/w), at most 8.0% (w/w), at most 8.5% (w/w), at most 9.0% (w/w), at most 9.5% (w/w), at most 10% (w/w), at most 15% (w/w), at most 20% (w/w), at most 25% (w/w), at most 30% (w/w), or at most 35% (w/w).

In some embodiments, the Clostridial toxin active ingredient pharmaceutical composition comprises a poloxamer. Poloxamers which can be used with the present pharmaceutical composition include Poloxamer 124 (PLURONIC® L44), Poloxamer 181 (PLURONIC® L61), Poloxamer 182 (PLURONIC® L62), Poloxamer 184 (PLURONIC® L64), Poloxamer 188 (e.g., PLURONIC® F68, KOLLIPHOR® P 188), Poloxamer 237 (PLURONIC® F87), Poloxamer 338 (PLURONIC® L108), Poloxamer 407 (PLURONIC® F127). In some embodiments, poloxamer 188 may be more advantageous.

In some embodiments, the Clostridial toxin active ingredient pharmaceutical composition comprises a polysorbate. Polysorbates which can be used with the present pharmaceutical composition includes polysorbate 20 (TWEEN® 20), polysorbate 40 (TWEEN® 40), polysorbate 60 (TWEEN® 60), polysorbate 61 (TWEEN® 61), polysorbate 65 (TWEEN® 65), polysorbate 80 (TWEEN® 80), and polysorbate 81 (TWEEN® 81). In some embodiments, polysorbate 20 may be more advantageous than some other polysorbates.

Aspects of the present pharmaceutical compositions provide, in part, at least an antioxidant. Non-limiting examples of antioxidant include, without limitation, methionine, cysteine, NAC, glutathionine, lipoic acid, sodium metabisulfite, sodium thiosulfate, ascorbic acid, butylated hydroxyanisole, butylated hydroxytoluene, vitamin E and analogs including Trolox C; chelators such as EDTA (ethylene diamine tetraacetic acid sodium salt), EGTA (ethylene glycol-bis(2-aminoethylether)-N,N,N′,N′-tetraacetic acid), DTPA (diethylenetriaminepentaacetic acid), analogues or derivatives thereof; and combinations thereof. In aspects of this embodiment, the amount of antioxidant added to the formulation ranges from about 0.01% (w/w) to about 0.10% (w/w).

It is further envisioned that a Clostridial toxin active ingredient pharmaceutical composition disclosed in the present specification can optionally include, without limitation, other pharmaceutically acceptable components (or pharmaceutical components), including, without limitation, buffers, preservatives, tonicity adjusters, salts, antioxidants, osmolality adjusting agents, emulsifying agents, sweetening or flavoring agents, and the like. Various buffers and means for adjusting pH can be used to prepare a pharmaceutical composition disclosed in the present specification, provided that the resulting preparation is pharmaceutically acceptable. Such buffers include, without limitation, acetate buffers, borate buffers, citrate buffers, phosphate buffers, neutral buffered saline, and phosphate buffered saline. It is understood that acids or bases can be used to adjust the pH of a pharmaceutical composition as needed. It is envisioned that any buffered pH level can be useful in formulating a Clostridial toxin active ingredient pharmaceutical composition, with the proviso that a therapeutically effective amount of the Clostridial toxin active ingredient is recovered using this effective pH level. In an aspect of this embodiment, an effective pH level is at least about pH 5.0, at least about pH 5.5, at least about pH 6.0, at least about pH 6.5, at least about pH 7.0 or at about pH 7.5. In another aspect of this embodiment, an effective pH level is at most about pH 5.0, at most about pH 5.5, at most about pH 6.0, at most about pH 6.5, at most about pH 7.0 or at most about pH 7.5. In yet another aspect of this embodiment, an effective pH level is about pH 5.0 to about pH 8.0, an effective pH level is about pH 5.0 to about pH 7.0, an effective pH level is about pH 5.0 to about pH 6.0, is about pH 5.5 to about pH 8.0, an effective pH level is about pH 5.5 to about pH 7.0, an effective pH level is about pH 5.5 to about pH 5.0, is about pH 5.5 to about pH 7.5, an effective pH level is about pH 5.5 to about pH 6.5.

The pharmaceutical compositions disclosed herein can have a pH of between about 5 and 8 when reconstituted or upon injection. In certain embodiments the composition will have a pH below 8, such as, for example, 7.9, or 7.8, or 7.7, or 7.6, or 7.5, or 7.4, or 7.3, or 7.2, or 7.1, or 7.0, or 6.9, or 6.8, or 6.7, or 6.6, or 6.5, or 6.4, or 6.3, or 6.2, or 6.1, or 6.0, or 5.9, or 5.8, or 5.7, or 5.6, or 5.5, or 5.4, or 5.3, or 5.2, or 5.1, or the like. In some embodiments, the pH ranges from 5 to 7.

It is envisioned that any concentration of a buffer can be useful in formulating a Clostridial toxin active ingredient pharmaceutical composition, with the proviso that a therapeutically effective amount of the Clostridial toxin active ingredient is recovered using this effective concentration of buffer. In aspects of this embodiment, an effective concentration of buffer is at least 0.1 mM, at least 0.2 mM, at least 0.3 mM, at least 0.4 mM, at least 0.5 mM, at least 0.6 mM, at least 0.7 mM, at least 0.8 mM, or at least 0.9 mM. In other aspects of this embodiment, an effective concentration of buffer is at least 1.0 mM, at least 2.0 mM, at least 3.0 mM, at least 4.0 mM, at least 5.0 mM, at least 6.0 mM, at least 7.0 mM, at least 8.0 mM, or at least 9.0 mM. In yet other aspects of this embodiment, an effective concentration of buffer is at least 10 mM, at least 20 mM, at least 30 mM, at least 40 mM, at least 50 mM, at least 60 mM, at least 70 mM, at least 80 mM, or at least 90 mM. In still other aspects of this embodiment, an effective concentration of buffer is at least 100 mM, at least 200 mM, at least 300 mM, at least 400 mM, at least 500 mM, at least 600 mM, at least 700 mM, at least 800 mM, or at least 900 mM. In further aspects of this embodiment, an effective concentration of buffer is at most 0.1 mM, at most 0.2 mM, at most 0.3 mM, at most 0.4 mM, at most 0.5 mM, at most 0.6 mM, at most 0.7 mM, at most 0.8 mM, or at most 0.9 mM. In still other aspects of this embodiment, an effective concentration of buffer is at most 1.0 mM, at most 2.0 mM, at most 3.0 mM, at most 4.0 mM, at most 5.0 mM, at most 6.0 mM, at most 7.0 mM, at most 8.0 mM, or at most 9.0 mM. In yet other aspects of this embodiment, an effective concentration of buffer is at most 10 mM, at most 20 mM, at most 30 mM, at most 40 mM, at most 50 mM, at most 60 mM, at most 70 mM, at most 80 mM, or at most 90 mM. In still other aspects of this embodiment, an effective concentration of buffer is at most 100 mM, at most 200 mM, at most 300 mM, at most 400 mM, at most 500 mM, at most 600 mM, at most 700 mM, at most 800 mM, or at most 900 mM. In still further aspects of this embodiment, an effective concentration of buffer is about 0.1 mM to about 900 mM, 0.1 mM to about 500 mM, 0.1 mM to about 100 mM, 0.1 mM to about 90 mM, 0.1 mM to about 50 mM, 1.0 mM to about 900 mM, 1.0 mM to about 500 mM, 1.0 mM to about 100 mM, 1.0 mM to about 90 mM, or 1.0 mM to about 50 mM.

Embodiments described herein can be practiced with a composition that comprises a plurality of botulinum toxin serotypes, such as botulinum toxin serotypes selected from the group consisting of botulinum toxin serotypes A, B, C₁ D, E, F and G. In certain embodiments, purified botulinum toxins, can be used. In other embodiments, modified botulinum toxins may be used.

In some embodiments, the composition may optionally also include NaCl. NaCl may particularly preferably be included in compositions comprising botulinum toxin, trehalose or sucrose, poloxamer 188, and methionine, and is particularly preferably included in liquid compositions comprising botulinum toxin, trehalose or sucrose, poloxamer 188, and methionine. In some lyophilized formulations, NaCl may function as a tonicity agent in a reconstitution vehicle. In one embodiment, NaCl may be present in an amount of 0.9% (w/w) in the reconstitution vehicle.

In some embodiments, the Clostridial toxin active ingredient pharmaceutical composition can be formulated as a lyophilized (i.e. freeze dried) or vacuum dried powder which can be reconstituted with a suitable fluid, such as saline or water, prior to administration to a patient. In alternative embodiments, the pharmaceutical composition can be formulated as an aqueous solution or suspension.

In some embodiments, the solid Clostridial toxin active ingredient pharmaceutical composition comprises a botulinum toxin, a non-cross-linked hyaluronic acid or salt thereof (such as from about 450 kDa to 2.0 MDa, such as from 450 kDa to 1.6 MDa, about 1.4-1.6 MDa and about 1400-1600 kDa, a tonicity agent and/or a lyoprotector, a poloxamer and/or a polysorbate and an antioxidant. In some embodiments, the Clostridial toxin active ingredient pharmaceutical composition comprises a botulinum toxin. In some embodiments, the Clostridial toxin active ingredient pharmaceutical composition comprises trehalose. In some embodiments, the Clostridial toxin active ingredient pharmaceutical composition comprises poloxamer 188 or polysorbate 20. In some embodiments, the composition comprises EDTA EGTA, DTPA, or analogs thereof. In alternative embodiments, the composition comprises methionine and/or NAC. In aspects of these alternative embodiments, the composition further comprises EDTA, EGTA, DTPA, or analogues thereof. In some embodiments, the composition further comprises a buffering agent. In one embodiment, the composition comprises histidine buffer. In some embodiments, the relative weight amounts of disaccharide, poloxamer and antioxidant are within the following ranges: trehalose: 1 to 15%, 1 to 10%, or 2-15% or 2-10%; poloxamer: 0.5-8% or 0.5 to 5%; methionine: 0.01 to 5%, 0.02 to 3%, 0.05 to 1%, 0.05 to 0.5%. In some embodiments, the relative weight amounts of trehalose, poloxamer and methionine are within the following ranges: trehalose: 1 to 15%, 1 to 10%, or 2-15% or 2-10%; poloxamer: 0.5-8% or 0.5 to 5%; methionine: 0.01 to 5%, 0.02 to 3%, 0.05 to 1%, 0.05 to 0.5%. In some embodiments, the relative weight amounts of trehalose, poloxamer and methionine are within the following ranges respectively: 1 to 10%; 0.5 to 5% and 0.1 to 0.3%. In other embodiments, the relative weight amounts of trehalose, polysorbate and methionine are within the following ranges respectively: 1 to 15%; 0.02% to 0.06%; and 0.1 to 0.3%. In other embodiments, the relative weight amounts of trehalose, polysorbate and methionine are within the following ranges respectively: 1 to 10%; 0.02% to 0.06%; and 0.1 to 0.3%. In some embodiments, the relative weight amount of EDTA or an EDTA analog is from about 0.01 to 0.10%. In some embodiments, the relative weight amount of NAC ranges from 0.01 to 0.5%.

In aspects of these embodiments, the Clostridial toxin active ingredient pharmaceutical composition is formulated as a solid (i.e., lyophilized or vacuum dried) composition. In some embodiments, the solid Clostridial pharmaceutical composition comprises a lyoprotector. In some embodiments, the preferred lyoprotector includes sucrose, trehalose, mannitol, sorbitol, glucose, or combinations thereof. In some embodiments, the solid pharmaceutical composition comprises NAC in a relative weight amount of 0.01 to 0.5%. In some embodiments, the pharmaceutical composition further comprises EDTA, EGTA, DTPA, or analogues thereof. In alternative embodiments, the solid pharmaceutical composition comprises methionine and EDTA, EGTA, DTPA, or analogues thereof.

In one embodiment, the composition is a solid composition consisting of botulinum toxin type A, 1.2% (w/w) non-cross-linked hyaluronic acid or salt thereof (weight average molecular weight is about 1.58 MDa), 8% (w/w) trehalose, 4% (w/w) poloxamer 188, 0.2% (w/w) methionine, and a Histidine buffer. In one embodiment, the solid composition is reconstituted with a reconstitution vehicle comprising NaCl prior to administration to a patient. In one embodiment, NaCl may be present in an amount of 0.9% (w/w) in the reconstitution vehicle.

In an alternative aspect of these embodiments, the Clostridial toxin active ingredient pharmaceutical composition is formulated as a liquid. In some embodiments, the liquid pharmaceutical composition comprises a tonicity agent selected from trehalose, sucrose, sodium chloride, mannitol, sorbitol, glucose, and combinations thereof. In some embodiments, the liquid pharmaceutical composition comprises NAC in a relative weight amount of 0.1 to 0.5%. In some embodiments the liquid pharmaceutical composition comprises NAC and a chelating agent selected from EDTA, EGTA, DTPA, and analogues thereof. In some embodiments, the liquid pharmaceutical composition comprises histidine buffer. In some embodiments, the liquid pharmaceutical composition has a pH from 5 to 7.

In one embodiment, the composition is a liquid composition consisting of botulinum toxin type A, 1.2% (w/w) non-cross-linked hyaluronic acid or salt thereof (weight average molecular weight is about 1.58 MDa), 8% (w/w) trehalose, 4% (w/w) poloxamer 188, 0.2% (w/w) methionine, and a Histidine buffer, pH 6.

### Methods of Treatment

In embodiments, methods of treating diseases, disorders, conditions, and the like, are described which comprise administering a pharmaceutical composition as described herein to a subject in need thereof in an amount sufficient to produce improved patient function. In certain embodiments, the diseases are of a neuromuscular nature, such as, for example, those diseases that affect muscles and nerve control thereof, such as, for example, overactive bladder, and the like. Certain embodiments relate to the treatment of pain, such as, for example, treatment of headache pain, or back pain, or muscle pain, or the like. In certain embodiments, methods encompass the treatment of psychological disorders, including, for example, depression, anxiety, and the like.

Compositions and methods described herein can be useful for the treatment, reduction of symptoms, and/or prevention of, for example, achalasia, anal fissure, anismus, blepharospasm, cerebral palsy, cervical dystonia, cervicogenic headache, hemifacial spasm, dyshidrotic eczema, dysphagia, dysphonia, esophageal dysmotility, esophageal muscular ring, esotropia (infantile), eyelift, facial myokemia, gait disturbances (idiopathic toe-walking), generalized dystonia, hemifacial spasm, hyperfunctional facial lines (glabellar, forehead, crows' feet, down-turned angles of the mouth), hyperhidrosis, incontinence (idiopathic or neurogenic), medication overuse headache, migraine headache, myoclonus, muscle mass or activity reduction, involving, for example, the masseter or the like, myofascial pain syndrome, obstructive urinary symptoms, pancreas divisum pancreatitis, Parkinson's disease, puborectalis syndrome, reduction of surgical scar tension, salivary hypersecretion, sialocele, sixth nerve palsy, spasticity, speech/voice disorders, strabismus, surgery adjunct (ophthalmic), tardive dyskinesia, temporomandibular joint disorders, tension headache, thoracic outlet syndrome, torsion dystonia, torticolis, Tourette's syndrome, tremor, whiplash-associated neck pain, pain, itching, inflammation, allergy, cancer and benign tumors, fever, obesity, infectious diseases, viral and bacterial, hypertension, cardiac arrhythmias, vasospasm, atherosclerosis, endothelial hyperplasia, venous thrombosis, varicose veins, apthous stomatitis, hypersalivation, temporomandibular joint syndrome, hyperhidrosis, bromhidrosis, acne, rosacea, hyperpigmention, hypertrophic scars, keloids, calluses and corns, skin wrinkling, excessive sebum production, psoriasis, dermatitis, allergic rhinitis, nasal congestion, post nasal drip, sneezing, ear wax, serous and suppurative otitis media, tonsil and adenoid hypertrophy, tinnitus, dizziness, vertigo, hoarseness, cough, sleep apnea, snoring, glaucoma, conjunctivitis, uveitis, strabismus, Grave's disease, excessive hair growth, hair loss, asthma, bronchitis, emphysema, mucus production, pleuritis, coagulation disorders, myeloproliferative disorders, disorders involving eosinophils, neutrophils, macrophages and lymphocytes, immune tolerance and transplantation, autoimmune disorders, dysphagia, acid reflux, hiatal hernia, gastritis and hyperacidity, diarrhea and constipation, hemorrhoids, urinary incontinence, prostatic hypertrophy, erectile dysfunction, priapism and Peyronie's disease, epididymitis, contraception, menstrual cramps, preventing premature delivery, endometriosis and fibroids, arthritis, osteoarthritis, rheumatoid, bursitis, tendonitis, tenosynovitis, fibromyalgia, seizure disorders, spasticity, headache, and neuralgias.

In certain embodiments, patients are limited to a maximum of 360 U of botulinum toxin administered over any 90-day period.

### Treatment of Nerve/Muscle Conditions

In an embodiment, the neuromuscular disease is hyperhidrosis. A subject suffering from hyperhidrosis, for example, receives about 59 U per axilla, or about 58 U per axilla, or about 57 U per axilla, or about 56 U per axilla, or about 55 U per axilla, or about 54 U per axilla, or about 53 U per axilla, or about 52 U per axilla, or about 51 U per axilla, or about 50 U per axilla, or about 49 U per axilla, or about 48 U per axilla, or about 47 U per axilla, or about 46 U per axilla, or about 45 U per axilla, or about 44 U per axilla, or about 43 U per axilla, or about 42 U per axilla, or about 41 U per axilla, or about 40 U per axilla, or about 39 U per axilla, or about 38 U per axilla, or about 37 U per axilla, or about 36 U per axilla, or less, per treatment of a pharmaceutical composition described herein. In an embodiment, 50 U total are injected intradermally into 10-15 sites spaced approximately 1-2 cm apart.

In an embodiment, the neuromuscular disease is hemifacial spasm. A subject suffering from hemifacial spasm, for example receives between about 1.5 to 15 U per treatment of a pharmaceutical composition described herein. In a further example, the subject receives between about 1.5 to 3 U, 1.5 to 5 U, 1.5 to 7 U, 1.5 to 10 U, 1.5 to 12 U, 1.5 to 15 U, 5 to 10 U, 5 to 15 U, or 10 to 15 U per treatment are administered to a patient with hemifacial spasm. In a still further example, the subject receives about 1.5 U, about 2 U, about 2.5 U, about 3 U, about 3.5 U, about 4 U, about 4.5 U about 5 U, about 5.5 U, about 6 U, about 6.5 U, about 7 U, about 7.5 U, about 8 U, about 8.5 U, about 9 U, about 9.5 U, about 10 U, about 10.5 U, about 11 U, about 11.5 U, about 12 U, about 12.5 U, about 13 U, about 13.5 U, about 14 U, about 14.5 U, or about 15 U per treatment are administered to a patient with hemifacial spasm. Dosages greater than 15 U per treatment may also be administered to patients with hemifacial spasm to achieve a therapeutic response. A treatment session can comprise multiple treatments.

In an embodiment, the neuromuscular disease is cervical dystonia. A subject suffering from cervical dystonia, for example, receives between about 15 to 300 U per treatment of a pharmaceutical composition described herein. In a further example, the subject receives between about 35 to 250 U, 65 to 200 U, 85 to 175 U, 105 to 160 U, or 125 to 145 U are administered to a patient with cervical dystonia. In an embodiment, dosages to the sternocleidomastoid muscle is limited to 100 U or less. Dosages greater than 300 U per treatment may also be administered to patients with cervical dystonia to achieve a therapeutic response. A treatment session can comprise multiple treatments.

In an embodiment, the neuromuscular disease is blepharospasm. A subject suffering from blepharospasm, for example, receives between about 1.25 to 2.5 U of a pharmaceutical composition described herein injected into the medial and lateral pretarsal orbicularis oculi of the upper lid and into the lateral pretarsal orbicularis oculi of the lower lid. In a further example, the subject receives about 1.5 U, about 1.6 U, about 1.7 U, about 1.8 U, about 1.9 U, about 2.0 U, about 2.1 U, about 2.2 U, about 2.3 U, about 2.4 U, about 2.5 U, or more, per injection site. A treatment session can comprise multiple treatments.

In an embodiment, the neuromuscular disease is strabismus. A subject suffering from strabismus, for example, receives between about 1.25 to 2.5 U per injection site of a pharmaceutical composition described herein. In a further example, the subject receives about 1.5 U, about 1.6 U, about 1.7 U, about 1.8 U, about 1.9 U, about 2.0 U, about 2.1 U, about 2.2 U, about 2.3 U, about 2.4 U, about 2.5 U, or more, per injection site to achieve a therapeutic response. In embodiments, lower doses are used for treatment of small deviations. In embodiments, vertical muscles and horizontal strabismus of less than 20 prism diameters can be treated with 1.25 to 2.5 U per injection site. A treatment session can comprise multiple treatments.

In an embodiment, the neuromuscular disease is muscle spasticity. A subject suffering from muscle spasticity, for example, receives between about 20 to 200 U per treatment of a pharmaceutical composition described herein. In a further example, the subject receives between about 20 to 30 U, 20 to 40 U, 20 to 60 U, 20 to 80 U, 20 to 100 U, 20 to 125 U, 20 to 150 U, or 20 to 175 U per treatment are administered to a patient with muscle spasticity. In a still further example, the subject receives about 20 U, about 25 U, about 30 U, about 35 U, about 40 U, about 45 U, about 50 U, about 55 U, about 60 U, about 65 U, about 70 U, about 75 U, about 80 U, about 85 U, about 90 U, about 95 U, about 100 U, about 105 U, about 110 U, about 115 U, about 120 U, about 125 U, about 130 U, about 135 U, about 140 U, about 145 U, about 150 U, about 155 U, about 160 U, about 165 U, about 170 U, about 175 U, about 180 U, about 185 U, about 190 U, about 195 U, or about 200 U per treatment are administered to a patient with muscle spasticity. In an embodiment, the biceps brachii can be injected with between 100 U and 200 U divided into 4 injection sites. In an embodiment, the flexor carpi radialis can be injected with between 12.5 U and 50 U in 1 injection site. In an embodiment, the flexor carpi ulnaris can be injected with between 12.5 U and 50 U in 1 injection site. In an embodiment, the flexor digitorum profundus can be injected with between 30 U and 50 U in one injection site. In an embodiment, the flexor digitorum sublimis can be injected with between 30 U and 50 in a single injection site. Dosages greater than 200 U per treatment may also be administered to patients with muscle spasticity to achieve a therapeutic response. A treatment session can comprise multiple treatments.

### Treatment of Pain

In another embodiment, methods for treating pain comprising the step of administering a pharmaceutical composition described herein to a subject in need thereof in an amount sufficient to reduce pain. In another embodiment, the patient suffers from myofascial pain, migraine headache pain, tension headache pain, neuropathic pain, facial pain, lower-back pain, sinus-headache pain, pain associated with temporomandibular joint disease, pain associated with spasticity or cervical dystonia, post-surgical wound pain, or neuralgia. A treatment session can comprise multiple treatments.

In an embodiment, the patient suffers from facial pain. A subject suffering from facial pain, for example, receives between about 4 to 40 U per treatment of a pharmaceutical composition described herein. In a further example, the subject receives between about 4 to 10 U, 4 to 15 U, 4 to 20 U, 4 to 25 U, 4 to 30 U, 4 to 35 U, 7 to 15 U, 7 to 20 U, 7 to 25 U, 7 to 30 U, 7 to 35 U, or 7 to 40 U per treatment are administered to a patient suffering from facial pain. In a still further example, the subject receives about 4 U, about 5 U, about 7.5 U, about 10 U, about 12.5 U, about 15 U, about 17.5 U, about 20.0 U, about 22.5 U, about 25.0 U, about 27.5 U, about 30.0 U, about 32.5 U, about 35 U, about 37.5 U, or about 40 U per treatment are administered to a patient with facial pain. Dosages greater than 40 U per treatment may also be administered to patients with facial pain to achieve a therapeutic response. A treatment session can comprise multiple treatments.

In an embodiment, the patient suffers from myofascial pain. A subject suffering from myofascial pain, for example, receives between about 5 to 100 U per treatment of a pharmaceutical composition described herein. In a further example, the subject receives between about 5 to 10 U, 5 to 20 U, 5 to 30 U, 5 to 40 Units, 5 to 50 Units, 5 to 60 Units, 5 to 70 Units, 5 to 80 Units, 5 to 90 U, 10 to 20 U, 10 to 30 U, 10 to 50 U, or 10 to 60 U, or 10 to 70 U, or 10 to 80 U, 10 to 90 U, or 10 to 100 U per treatment are administered to a patient suffering from myofascial pain. In a further example, the subject receives about 5 U, about 10 U, about 15 U, about 20 U, about 25 U, about 30 U, about 35 U, about 40 U, about 45 U, about 50 U, about 55 U, about 60 U, about 65 U, about 70 U, about 75 U, about 80 U, about 85 U, about 90 U, about 95 U, or about 100 U per treatment. Dosages greater than 100 U per treatment may also be administered to patients with myofascial pain to achieve a therapeutic response. A treatment session can comprise multiple treatments.

In an embodiment, the subject suffers from lower-back pain. A subject suffering from lower-back pain, for example, receives between about 15 to 150 U per treatment of a pharmaceutical composition described herein. In a further example, the subject receives between about 15 to 30 U, 15 to 50 U, 15 to 75 U, 15 to 100 U, 15 to 125 U, 15 to 150 U, 20 to 100 U, 20 to 150 U, or 100 to 150 U per treatment. In a still further example, the subject receives about 15 U, about 20 U, about 25 U, about 30 U, about 35 U, about 40 U, about 45 U, about 50 U, about 55 U, about 60 U, about 65 U, about 70 U, about 75 U, about 80 U, about 85 U, about 90 U, about 95 U, about 100 U, about 105 U, about 110 U, about 115 U, about 120 U, about 125 U, about 130 U, about 135 U, about 140 U, about 145 U, or about 150 U per treatment to alleviate lower-back pain. Dosages greater than 150 U per treatment may also be administered to patients with lower-back pain to achieve a therapeutic response. A treatment session can comprise multiple treatments.

In an embodiment, the patient suffers from migraine headache pain, including wherein the patient suffers from migraine headaches of 4 hours or more 15 or more days per month. A subject suffering from migraine-headache pain, for example, receives between about 0.5 to 200 U per treatment of a pharmaceutical composition described herein. In a further example, the subject receives between about 5 to 190 U, 15 to 180 U, 25 to 170 U, 35 to 160 U, 45 to 150 U, 55 to 140 U, 65 to 130 U, 75 to 120 U, 85 to 110 U, or 95 to 105 U per treatment to alleviate migraine headache pain. A treatment session can comprise multiple treatments.

For example, about 0.5 U, about 1.0 U, about 1.5 U, about 2.0 U, about 2.5 U, about 3.0 U, about 3.5 U, about 4.0 U, about 4.5 U, about 5.0 U, about 5.5 U, about 6.0 U, about 6.5 U, about 7.0 U, about 7.5 U, about 8.0 U, about 8.5 U, about 9.0 U, about 9.5 U, about 10.0 U, about 12 U, about 15 U, about 17 U, about 20 U, about 22 U, about 25 U, about 27 U, about 30 U, about 32 U, about 35 U, about 37 U, about 40 U, about 42 U, about 45 U, about 47 U, or about 50 U per treatment site are administered to a patient with migraine-headache pain. A patient can be treated at multiple sites, such as, for example, 2 sites, 3 sites, 4 sites, 5 sites, 6 sites, 7 sites, 8 sites, 9 sites, 10 sites, 11 sites, 12 sites, 13 sites, 14 sites, 15 sites, 16 sites, 17 sites, 18 sites, 19 sites, 20 sites, 21 sites, 22 sites, 23 sites, 24 sites, 25 sites, 26 sites, 27 sites, 28 sites, 29 sites, 30 sites, 31 sites, 32 sites, or more, or the like. In an embodiment, a patient suffering from migraine is injected 31 times with 5 U per 0.1 mL injection, across the corrugator (2 injections of 5 U each), procerus (1 injection of 5 U), frontalis (4 injections of 5 U each), temporalis (8 injections of 5 U each), occipitalis (6 injections of 5 U each), cervical paraspinal (4 injections of 5 U each), and trapezius (6 injections of 5 U each) muscles. With the exception of the procerus muscle which can be injected at the midline, all muscles can, in certain embodiments, be injected bilaterally with half of the injection sites to the left and half to the right side of the head and neck. Dosages greater than 200 U per treatment may also be administered to patients with migraine-headache pain to achieve a therapeutic response. A treatment session can comprise multiple treatments.

In an embodiment, the patient suffers from sinus-headache pain. A subject suffering from sinus-headache pain, for example, receives between about 4 to 40 U per treatment of a pharmaceutical composition described herein. In a further example, the subject receives between about 4 to 10 U, 4 to 15 U, 4 to 20 U, 4 to 25 U, 4 to 30 U, 4 to 35 U, 7 to 15 U, 7 to 20 U, 7 to 25 U, 7 to 30 U, 7 to 35 U, or 7 to 40 U per treatment to alleviate sinus-headache pain. In a still further example, the subject receives about 4 U, about 5 U, about 7.5 U, about 10 U, about 12.5 U, about 15 U, about 17.5 U, about 20.0 U, about 22.5 U, about 25.0 U, about 27.5 U, about 30.0 U, about 32.5 U, about 35 U, about 37.5 U, or about 40 U per treatment. Dosages greater than 40 U per treatment may also be administered to patients with sinus headache-pain to achieve a therapeutic response. A treatment session can comprise multiple treatments.

In an embodiment, the patient suffers from tension-headache pain. A subject suffering from tension-headache pain, for example, receives between about 5 to 50 U per treatment of a pharmaceutical composition described herein. In a further example, between about 5 to 10 U, 5 to 15 U, 5 to 20 U, 5 to 25 U, 5 to 30 U, 5 to 35 U, 5 to 40 U, 5 to 45 U, 10 to 20 U, 10 to 25 U, 10 to 30 U, 10 to 35 U, 10 to 40 U, or 10 to 45 U per treatment are administered to a patient with tension-headache pain. In a still further example, the subject receives about 5 U, about 10 U, about 20 U, about 25 U, about 30 U, about 35 U, about 40 U, about 45 U, or about 50 U per treatment administered to alleviate tension-headache pain. In an embodiment, a patient suffering from tension headache is injected 31 times with 5 U per 0.1 mL injection, across the corrugator (2 injections of 5 U each), procerus (1 injection of 5 U), frontalis (4 injections of 5 U each), temporalis (8 injections of 5 U each), occipitalis (6 injections of 5 U each), cervical paraspinal (4 injections of 5 U each), and trapezius (6 injections of 5 U each) muscles. With the exception of the procerus muscle which can be injected at the midline, all muscles can, in certain embodiments, be injected bilaterally with half of the injection sites to the left and half to the right side of the head and neck. Dosages greater than 200 U per treatment may also be administered to patients with tension headache pain to achieve a therapeutic response. A treatment session can comprise multiple treatments.

In an embodiment, the patient suffers from sinus headache pain or facial pain associated with acute or recurrent chronic sinusitis. For example a pharmaceutical composition as described herein can be administered to the nasal mucosa or to the subcutaneous structures overlying the sinuses, wherein the administration of the formulation reduces the headache and/or facial pain associated with acute recurrent or chronic sinusitis. In further embodiments, any of the pharmaceutical formulations described herein can be administered to the nasal mucosa or to the subcutaneous structures overlying the sinuses, such as over one or more of the sinuses selected from the group consisting of: ethmoid; maxillary; mastoid; frontal; and sphenoid. In another embodiment, subcutaneous structures overlying the sinuses lie within one or more of the areas selected from the group consisting of: forehead; malar; temporal; post auricular; and lip. In embodiments, multiple injections of 5 U each are administered to treat the sinus headache pain or facial pain associated with acute or recurrent chronic sinusitis.

In another embodiment, a patient suffering from sinus headache pain or facial pain associated with acute or recurrent chronic sinusitis is treated by administering any of the pharmaceutical formulations described herein to an afflicted area of the patient. In a further embodiment, the pharmaceutical formulations disclosed herein are administered to the projections of a trigeminal nerve innervating a sinus.

Patients suffering from sinus headache pain or facial pain associated with acute or recurrent chronic sinusitis often exhibit symptoms including rhinitis, sinus hypersecretion and/or purulent nasal discharge. In one embodiment, patients treated with the pharmaceutical compositions described herein exhibit, prior to treatment, symptoms of sinus hypersecretion and purulent nasal discharge.

Embodiments contemplated herein provide methods for treating a patient suffering from sinus headache pain or facial pain associated with acute or recurrent chronic sinusitis, wherein the subject suffers from neuralgia. In certain embodiments the neuralgia is trigeminal neuralgia. In another embodiment, the neuralgia is: associated with compressive forces on a sensory nerve; associated with intrinsic nerve damage, demyelinating disease, or a genetic disorder; associated with a metabolic disorder; associated with central neurologic vascular disease; or associated with trauma. In another embodiment, the pain is associated with dental extraction or reconstruction.

### Treatment of Urological Disorders

In an embodiment, methods for treating a patient suffering from overactive bladder (OAB), such as, for example, that due to a neurologic condition (NOAB), or idiopathic OAB (IOAB) are provided. For example, pharmaceutical formulations described herein can be administered to the bladder or its vicinity, e.g. the detrusor, wherein the administration of the formulation reduces the urge incontinence associated with overactive bladder. In certain embodiments, the dosage can be, for example, 200 U, or more, or less, or the like. For example, the dosage can be about 15 U, about 20 U, about 25 U, about 30 U, about 35 U, about 40 U, about 45 U, about 50 U, about 55 U, about 60 U, about 65 U, about 70 U, about 75 U, about 80 U, about 85 U, about 90 U, about 95 U, about 100 U, about 105 U, about 110 U, about 115 U, about 120 U, about 125 U, about 130 U, about 135 U, about 140 U, about 145 U, about 150 U, about 160 U, about 170 U, about 180 U, about 190 U, about 200 U, about 210 U, about 220, about 230 U, about 240 U, or more, or the like, per treatment. A patient can be injected at multiple sites, such as, for example, 2 sites, 3 sites, 4 sites, 5 sites, 6 sites, 7 sites, 8 sites, 9 sites, 10 sites, 11 sites, 12 sites, 13 sites, 14 sites, 15 sites, 16 sites, 17 sites, 18 sites, 19 sites, 20 sites, 21 sites, 22 sites, 23 sites, 24 sites, 25 sites, 26 sites, 27 sites, 28 sites, 29 sites, 30 sites, 31 sites, 32 sites, 33 sites, 34 sites, 35 sites, 36 sites, 37 sites, 38 sites, or more, or the like. In an embodiment, patients suffering from OAB are treated with 30 1 mL injections of approximately 6.7 U per injection into the detrusor muscle.

In an embodiment, methods for treating a patient suffering from neurogenic detrusor overactivity (NDO), such as that due to a neurologic condition, are provided. For example, pharmaceutical formulations can be administered to the bladder or its vicinity, e.g. the detrusor, wherein the administration of the formulation reduces the urge incontinence associated with overactive bladder. In certain embodiments, the dosage can be, for example, 200 U, or more, or less, or the like. For example, the dosage can be about 15 U, about 20 U, about 25 U, about 30 U, about 35 U, about 40 U, about 45 U, about 50 U, about 55 U, about 60 U, about 65 U, about 70 U, about 75 U, about 80 U, about 85 U, about 90 U, about 95 U, about 100 U, about 105 U, about 110 U, about 115 U, about 120 U, about 125 U, about 130 U, about 135 U, about 140 U, about 145 U, about 150 U, about 160 U, about 170 U, about 180 U, about 190 U, about 200 U, about 210 U, about 220, about 230 U, about 240 U, or more, or the like, per treatment. A patient can be injected at multiple sites, such as, for example, 2 sites, 3 sites, 4 sites, 5 sites, 6 sites, 7 sites, 8 sites, 9 sites, 10 sites, 11 sites, 12 sites, 13 sites, 14 sites, 15 sites, 16 sites, 17 sites, 18 sites, 19 sites, 20 sites, 21 sites, 22 sites, 23 sites, 24 sites, 25 sites, 26 sites, 27 sites, 28 sites, 29 sites, 30 sites, 31 sites, 32 sites, or more, or the like. In an embodiment, patients suffering from NDO are treated with 30 1 mL injections of approximately 6.7 U per injection into the detrusor muscle.

### Treatment of Cosmetic Features

In another embodiment, methods for cosmetically modifying soft-tissue features comprising the step of administering at least one pharmaceutical composition as described herein to a subject in need thereof in an amount sufficient to modify said features are provided. In a further embodiment, the pharmaceutical composition is administered via transcutaneous or transmucosal injection either at a single focus or multiple foci.

In embodiments, pharmaceutical formulations are administered to the face or neck of the subject. In a further embodiment, the pharmaceutical formulations are administered to the subject in an amount sufficient to reduce rhytides. For example, the formulation can be administered between eyebrows of the subject in an amount sufficient to reduce vertical lines between the eyebrows and on a bridge of a nose. The pharmaceutical formulations can also be administered near either one or both eyes of the subject in an amount sufficient to reduce lines at corners of the eyes. In an embodiment, compositions can be injected locally to smooth skin. In another embodiment, the pharmaceutical formulations can also be administered to a forehead of the subject in an amount sufficient to reduce horizontal lines on said forehead. In yet another embodiment, the pharmaceutical formulation is administered to the neck of the subject in an amount sufficient to reduce muscle bands in the neck. In an embodiment, a pharmaceutical composition is applied to the masseter muscle to relax the muscle and/or decrease masseter mass.

In a further embodiment, the patient suffers from facial wrinkles. A subject suffering from facial wrinkles, for example, can receive between about 1 to 100 U per treatment of a pharmaceutical formulation. In a further example, the subject receives between about 1 to 10 U, 1 to 20 U, 1 to 30 U, 1 to 40 U, 1 to 50 U, 1 to 60 U, 1 to 70 U, 1 to 80 U, 1 to 90 U, 5 to 20 U, 5 to 30 U, 5 to 40 U, 5 to 50 U, 5 to 60 U, 5 to 70 U, 5 to 80 U, 5 to 90 U, or 5 to 100 U per treatment. In a still further example, the subject receives about 1 U, about 10 U, about 20 U, about 30 U, about 40 U, about 50 U, about 60 U, about 70 U, about 80 U, about 90 U, or about 100 U per treatment. Dosages greater than 100 U per treatment may also be administered to patients suffering from inflammation or an inflammatory disorder to achieve a therapeutic response.

### Treatment of Inflammation

In another embodiment, methods for treating inflammation comprising the step of administering a pharmaceutical composition as described herein to a subject in need thereof in an amount sufficient to reduce inflammation. In certain embodiments, pharmaceutical formulations are administered to a patient without producing muscle weakness. In an embodiment, the pharmaceutical formulations are administered to patients with an inflammatory condition. In certain embodiments the inflammatory condition is neurogenic inflammation. In another embodiment, the subject suffers from rheumatoid arthritis or a gastro-intestinal inflammatory disease.

In a further embodiment, the patient suffers from an inflammatory disorder. A subject suffering from an inflammatory disorder, for example, receives between about 1 to 100 U per treatment of a pharmaceutical composition as described herein. In a further example, the subject receives between about 1 to 10 U, 1 to 20 U, 1 to 30 U, 1 to 40 U, 1 to 50 U, 1 to 60 U, 1 to 70 U, 1 to 80 U, 1 to 90 U, 5 to 20 U, 5 to 30 U, 5 to 40 U, 5 to 50 U, 5 to 60 U, 5 to 70 U, 5 to 80 U, 5 to 90 U, or 5 to 100 U per treatment. In a still further example, the subject receives about 1 U, about 10 U, about 20 U, about 30 U, about 40 U, about 50 U, about 60 U, about 70 U, about 80 U, about 90 U, or about 100 U per treatment. Dosages greater than 100 U per treatment may also be administered to patients suffering from inflammation or an inflammatory disorder to achieve a therapeutic response.

### Treatment of Skin Conditions

A method for treating a skin disorder can have the step of local administration of a botulinum neurotoxin to a location of a skin disorder of a patient, such as to a face, hand or foot of a patient. The neurotoxin can be locally administered in an amount of between about 10⁻³ units/kg of patient weight and about 35 units/kg of patient weight. For example, the neurotoxin is locally administered in an amount of between about 10⁻² U/kg and about 25 U/kg of patient weight. In a further example, the neurotoxin is administered in an amount of between about 10⁻¹ U/kg and about 15 U/kg. In one method, the neurotoxin is locally administered in an amount of between about 1 U/kg and about 10 U/kg in a composition as described herein. In a clinical setting it can be advantageous to administer from 1 U to 3000 U of a neurotoxin, such as botulinum toxin type A or B, to a skin disorder location by topical application or by subdermal administration, to effectively treat the skin disorder.

Administration of botulinum toxin can be carried out at multiple sites in the skin, wherein the sites of adjacent injections are separated by about 0.1 to 10 cm, or about 0.5 to about 5 cm, for example, by about 1.5 to about 3 cm. The toxins may be any of the botulinum toxins A, B, C, D, E, F, G or a mosaic toxin. The amounts administered may vary between 0.1 and 1000 U, or about 1 to about 40, or from about 5 to about 10 U, depending on the manufactures specifications, the class of the toxin and the mode of administration. The repeat time range for these administrations for maintenance of the desired change varies substantially according to the location of the injection, the condition to be adjusted and the condition of the patient. Thus, the repeat time may vary from about 1 week to about 50 weeks, however, a common range is about 4 to about 25 weeks, or even about 12 weeks to about 16 weeks.

The distances between administration sites, such as, for example, injection sites, can vary from about 1 mm to about 10 cm, suitably from about 5 mm to about 5 cm, and more usually from about 1 cm to about 3 cm. Thus for example botulinum A may be suitably administered by intradermal injection between about 0.1 to about 10 U at a separation of from about 0.5 to about 10 cm.

In another embodiment, methods for treating cutaneous disorders comprising the step of administering a pharmaceutical composition as described herein to a subject in need thereof in an amount sufficient to reduce a sebaceous or mucous secretion is provided. In further embodiments, the pharmaceutical compositions as described herein are administered to a patient without producing muscle weakness. In certain embodiments the pharmaceutical composition as described herein are injected into one or more sites of an eyelid or conjunctiva. In another embodiment, the formulations are administered to a body surface.

In another embodiment, the pharmaceutical formulations are administered in an amount sufficient to reduce cutaneous bacterial or fungal growth, including but not limited to Staphylococcus; Streptococcus and Moraxella. For example, the pharmaceutical compositions as described herein are administered to an area selected from the group consisting of: eyelid; scalp; feet; groin; and armpit to reduce cutaneous infection.

### Treatment of Depression

In another embodiment, a method for treating depression is provided. Depression is a general term for recognized forms of depression that are defined and are separately diagnosed according to criteria given in handbooks for psychiatry, for example in the Diagnostic and Statistical Manual of Mental Disorders 4th edition (DSM-IV) published by the American Psychiatric Association, Washington, D.C. (1994). In the DSM-IV, depressive disorders are classified under mood disorders and are divided into three types: major depressive disorder, dysthymic disorder and depressive disorder not otherwise specified (or “atypical”). In general, regardless of whether or not the depressive syndrome is melancholic, atypical, or some admixture of the two, a diagnosis of major depression is given when depressed mood is present, or loss of interest or pleasure in all activities is present, for at least two weeks.

Depression is often associated with psychomotor abnormalities, such as increased or retarded motor activity. Many depressed persons can also be recognized by their “depressed facies” in which the muscles of facial expression assume a distressed or sad appearance. For example, the brow may be furrowed, the inner ends of the eyebrows raised, and the angles of the mouth lowered such that the facial appearance is recognizably sad and/or anxious. Four major muscle groups are involved in frowning: the frontail, procerus, corrugator supercilii and orbicularis oculi (Weider et al. Dern Surg. 24:1172-1174, 1998. The corrugator supercilii is also known as the “scowl” muscle.

A subject diagnosed with depression or experiencing a depressive episode is treated by administering any of the pharmaceutical compositions described herein. In a further embodiment, the pharmaceutical formulations disclosed herein are administered to the patient via injection subcutaneously. Example 10 describes treatment of a person with a form of depression by administering a therapeutically effective amount of botulinum toxin in a composition comprising botulinum toxin, trehalose, a poloxamer surfactant, and methionine. The composition is administered to a facial muscle involved in frowning or scowling. The neurotoxin affects the ability of the subject to frown and/or scowl, thereby treating depression. Moe generally, a therapeutically effective amount of Botulinum toxin A can be injected into one or more of the frontalis, procerus, the corrugator supercilii, orbicularis oculi, or the depressor anguli oris (trianglaris muscle).

In another embodiment, the method comprises administering a therapeutically effective amount of a Clostridial toxin active ingredient in a composition as described herein to a facial muscle involved in frowning, scowling, or a sad appearance. The Clostridial toxin active ingredient causes partial or complete paralysis of the facial muscle, thereby affecting the ability of the subject to frown and/or scowl, or appear sad, and thereby treat depression. For example, a therapeutically effective amount of a composition comprising the Clostridial toxin active ingredient botulinum toxin, along with a surfactant, an antioxidant, and optionally a tonicity modifier, can be injected into one or more of the orbicularis oculi, frontalis, procerus, the corrugator supercilii, or the depressor anguli oris (trianglaris muscle). In a specific example, the composition comprising Botulinum toxin A is injected into the procerus muscle over the glabella. Other administration points and paradigms are disclosed, for example, in U.S. Pat. No. 7,758,872, which is incorporated by reference herein.

In other embodiments of the method, adult subjects with moderate to severe major depressive disorder (MDD), either single episode or recurrent are contemplated for treatment, where the MDD diagnosis is based upon the DSM-IV-TR criteria. In one embodiment, a single treatment is contemplated, and in other embodiments, a single, repeated treatment is contemplated, with the treatment is repeated at intervals of 2-6, 2-4, or 3-6 months. The amount of Clostridial toxin active ingredient dosed is, for example, 30 U or 50 U, where, in some embodiments, the dose is divided into a plurality of injections. In one embodiment, the plurality of injections is 6 and in another embodiment is 8. In one embodiment, 30 U is divided into 6 injections to the glabellar region of the forehead (procerus and corrugator muscles). In one embodiment, 50 U is divided into 8 injections to the glabellar region of the forehead (procerus and corrugator muscles).

Effective treatment is indicated by, for example, a primary efficacy measure known in the art, such as the clinical assessment known as Montgomery-Asberg Depression Rating scale. Additional efficacy measures: clinic CGI-S score (Clinical Global Impression of Change scores), clinic HAM-D17 total score (Hamilton Rating Scale for Depression).

### Treatment of Cardiac Arrhythmia

In another embodiment, a method for treating cardiac arrhythmia is provided. Arrhythmias are caused by a disruption of the normal functioning of the electrical conduction system of the heart. Normally, the chambers of the heart (atria and ventricles) contract in a coordinated manner. The signal to contract is an electrical impulse that begins in the sinoatrial nod, and the impulse is conducted through the atria and stimulates them to contract. The impulse passes through the atrioventricular node, then travels through the ventricles and stimulates them to contract. Problems can occur anywhere along the conduction system, causing various arrhythmias. Problems can also occur in the heart muscle itself, causing it to respond differently to the signal to contract, also causing arrhythmias, or causing the ventricles to contract independently of the normal conduction system.

Arrhythmias include tachycardias, bradycardias and true arrhythmias of disturbed rhythm. Arrhythmias are classified as lethal if they cause a severe decrease in the pumping function of the heart. When the pumping function is severely decreased for more than a few seconds, blood circulation is essentially stopped, and organ damage (such as brain damage) can occur within a few minutes. Lethal arrhythmias include ventricular fibrillation, also ventricular tachycardia that is rapid and sustained, or pulseless, and may include sustained episodes of other arrhythmias. Additional types of arrhythmias include atrial fibrillation or flutter, multifocal atrial tachycardia, paroxysmal supraventricular tachycardia, Wolff-Parkinson-White syndrome, sinus tachycardia, sinus bradycardia, bradycardia associated with heart block, sick sinus syndrome, and ectopic heartbeat.

Accordingly, a method for treating cardiac arrhythmia is provided, the method comprising the step of administering a composition as described herein that comprises a therapeutically effective amount of Clostridial toxin active ingredient, the composition administered locally to the heart of a patient with a cardiac arrhythmia or at risk of a cardiac arrhythmia. Particular arrhythmias treatable include bradycardia and tachycardia. In one embodiment, the composition is locally administered, by which is meant administration directly to, in, or to the vicinity of, the cardiac muscle to be treated. Local administration includes intrapericardial, intracardiac cardiac catheterization and direct cardiac muscle injection routes of administration for the composition.

Example 4 describes treatment of a person undergoing cardiac surgery by administering a therapeutically effective amount of botulinum toxin in a composition comprising botulinum toxin, trehalose, a poloxamer surfactant, and NAC. In one embodiment, the composition is administered via injection into one or more epicardial fat pads of the heart. The dose administered, in one exemplary embodiment, 25 U per epicardial fat pad, to a total dose of 125 U. In another exemplary embodiment, 50 U per epicardial fat pad, to a total dose of 250 U, is administered.

Effective treatment is indicated by, for example, a primary efficacy endpoint of, for example, incidence of atrial fibrillation (AF) as measured by ECG for 4 weeks or at 4 weeks post treatment. Additional efficacy endpoints include length of hospital stay, length of stay in ICU, rehospitalization rate, anticoagulant medication use, need for interventional procedures for post-operative atrial fibrillation, such as ablation, pacemaker implantation, electrical or pharmacologic cardioversion.

### EXAMPLES

The following examples illustrate embodiments and aspects of the present compositions and methods and are not intended to limit the scope thereof.

### Example 1

### Manufacturing and Testing of Hyaluronic Acid (HA) Containing Botulinum Toxin Compositions

Botulinum toxin-hyaluronic acid formulations were prepared with different molecular weight and intrinsic viscosity sodium hyaluronate at different concentrations as follows. Non-cross-linked sodium hyaluronate powder, of various molecular weights and intrinsic viscosities, was dissolved in a solution of 20 mM histidine, pH 6, 8% trehalose, 4% poloxamer 188, and 0.2% methionine. Table 1 above summarizes the components and amount of each component in the compositions. The viscosity of each composition was measured using an Anton Parr Rheometer, at shear rate that varied from 0.01/s to 100/s, with values reported at a shear rate of 0.1/second, and at 25° C.

A solution comprising botulinum toxin type A (BoNT/A) was mixed into each of the hyaluronic acid compositions of Table 1, to form compositions of botulinum toxin and linear, non-cross-linked hyaluronic acid with various concentrations of botulinum toxin. Table 2 above summaries the compositions prepared. Several compositions without non-cross-linked hyaluronic acid were prepared for comparative controls. The compositions were stored at 2-8° C. for about 48 hours to allow entrained air to dissipate prior to dosing in vivo.

### Example 2

### Expended Duration of Botulinum Toxin in Compositions Comprising a Non-Cross-Linked Hyaluronic Acid

Botulinum toxin-hyaluronic acid compositions and the comparative control compositions prepared in Example 1 were tested in vivo using the rat DAS (digital abduction score) assay to assess duration of effect. A test or control composition was injected (50 μL) intramuscularly into the tibialis anterior of the rat using a 25 G needle. Rat paralysis was assessed by the DAS response using a score from 0 to 4, with 4 representing maximum paralysis. The results are shown in FIGS. 1A-1C.

### Example 3

### Extended Duration of Botulinum Toxin in Presence of Non-Cross-Linked Hyaluronic Acid

Botulinum toxin serotype A solution was prepared using the commercially available product BOTOX® by reconstituting the powdered botulinum toxin with isotonic saline (0.9% sodium chloride). This BoNT/A solution was loaded into a syringe for use as a control. A BoNT/A solution was syringe mixed with a formulation comprising 1.2 wt % non-cross-linked sodium hyaluronate (average MW 1500 kDa) in 20 mM histidine, pH 6, 8% trehalose, 4% poloxamer 188, and 0.2% methionine (see Table 1 above). The test and control compositions were injected injected (50 μL) intramuscularly into the tibialis anterior of the rat using a 25 G needle. Rat paralysis was assessed by the DAS response using a score from 0 to 4, with 4 representing maximum paralysis. The results are shown in FIG. 2.

### Example 4

### Method for Treating Cardia Arrhythmia

A formulation comprised of botulinum toxin type A (BoNT/A, 50 U), 1.2 wt % HA or salt thereof (weight average molecular weight is about 1.58 MDa), 2 wt % trehalose, 4 wt % poloxamer P188, 0.3 wt % NAC, in 20 mM histidine buffer, pH 5.5 is provided.

A Caucasian male is undergoing cardiac surgery. As part of the surgical procedure, an amount of the reconstituted composition to provide a total dose of 75 U botulinum toxin is evenly divided for administration via injection into three epicardial fat pads of his heart. The medical staff reports no arrhythmia during or post-surgery.

Many alterations and modifications may be made by those having ordinary skill in the art, without departing from the spirit and scope of the disclosure. Therefore, it must be understood that the described embodiments have been set forth only for the purposes of examples, and that the embodiments should not be taken as limiting the scope of the following claims. The following claims are, therefore, to be read to include not only the combination of elements which are literally set forth, but all equivalent elements for performing substantially the same function in substantially the same way to obtain substantially the same result. The claims are thus to be understood to include those that have been described above, those that are conceptually equivalent, and those that incorporate the ideas of the disclosure.

What is claimed is:

1. A pharmaceutical composition, comprising:
(i) a Clostridial toxin active ingredient; and
(ii) non-crosslinked hyaluronic acid or salt thereof,
wherein the weight average molecular weight of the non-crosslinked hyaluronic acid or salt thereof is from 250 kDa to 2.4 MDa or from 4.6 MDa to 8 MDa, and wherein the pharmaceutical composition does not comprise crosslinked hyaluronic acid or salt thereof.

2. The pharmaceutical composition of claim 1, further comprising:
(i) a tonicity agent selected from trehalose, sucrose, sodium chloride, mannitol, sorbitol, glucose, and combinations thereof;
(ii) a surfactant selected from a poloxamer, a polysorbate, and combinations thereof; and
(iii) an antioxidant selected from methionine, N-acetyl cysteine, ethylenediaminetetraacetic acid, and combinations thereof.

3. The pharmaceutical composition of claim 1, wherein the Clostridial toxin active ingredient is a botulinum toxin.

4. The pharmaceutical composition of claim 3, wherein the tonicity agent is trehalose.

5. The pharmaceutical composition of claim 4, wherein trehalose is present in an amount between about 1 to 15 wt %.

6. The pharmaceutical composition of claim 3, wherein the surfactant is a poloxamer.

7. The pharmaceutical composition of claim 6, wherein the poloxamer is present in an amount between about 0.5 to 8 wt %.

8. The pharmaceutical composition of claim 3, wherein the antioxidant is methionine.

9. The pharmaceutical composition of claim 8, wherein methionine is present in an amount between about 0.01 to 0.5 wt %.

10. The pharmaceutical composition of claim 3, wherein the composition comprises trehalose, poloxamer 188 and methionine.

11. The pharmaceutical composition claim 3, wherein the composition comprises about 8 w/w % trehalose, 4 w/w % poloxamer 188 and 0.2 w/w % methionine.

12. The pharmaceutical composition of claim 1, wherein the weight average molecular weight of the hyaluronic acid or salt thereof is from about 450 kDa to 2.0 MDa,

13. The pharmaceutical composition of claim 1, wherein the non-cross-linked hyaluronic acid or salt thereof is present in an amount from about 0.2 to 10 wt %.

14. The pharmaceutical composition of claim 13, wherein the weight average molecular weight of the hyaluronic acid or salt thereof is between about 1.5-1.6 MDa.

15. The pharmaceutical composition of claim 1, wherein the composition comprises up to 10 Units of Clostridial toxin per milligram of a non-cross-linked hyaluronic acid or salt thereof.

16. A pharmaceutical composition of claim 1, comprising:
(i) a Clostridial toxin active ingredient;
(ii) a non-cross-linked hyaluronic acid or salt thereof with weight average molecular weight of from 450 kDa to 2.0 MDa;
(ii) trehalose in an amount between about 1-15 wt %;
(iii) poloxamer in an amount between about 0.5-8 wt %; and
(iv) methionine in an amount between about 0.05-5 wt %.

17. The pharmaceutical composition of claim 16, wherein the Clostridial toxin active ingredient is a botulinum toxin.

18. A pharmaceutical composition comprising:
(i) a non-crosslinked hyaluronic acid or salt thereof,
(ii) a tonicity agent selected from trehalose, sucrose, sodium chloride, mannitol, sorbitol, glucose, and combinations thereof;
(iii) a surfactant selected from a poloxamer, a polysorbate, and combinations thereof; and
(iv) an antioxidant selected from methionine, N-acetyl cysteine, ethylenediaminetetraacetic acid, and combinations thereof;
wherein the weight average molecular weight of the non-crosslinked hyaluronic acid or salt thereof is from about 450 kDa to 2.0 MDa, and wherein the pharmaceutical composition does not comprise crosslinked hyaluronic acid or salt thereof.

19. The pharmaceutical composition of claim 18, wherein the Clostridial toxin active ingredient is a botulinum toxin.

20. A method of treating glabellar lines, comprising:
providing for administration the composition of claim 1.
